SEQUENCE LISTING (601 sequences)

SEQ ID NO: 1 - RNA, 23 nt, artificial Sequence
  sequences of Human Parecho virus PSs
aggggggaucccugguuuccuuu

SEQ ID NO: 2 - RNA, 19 nt, artificial Sequence
  Sequences of Human Parecho virus PSs
uuccacauguuuugaugaa

SEQ ID NO: 3 - RNA, 22 nt, artificial Sequence
  Sequences of Human Parecho virus PSs
ugaauguuuuuguuaacaguua

SEQ ID NO: 4 - RNA, 22 nt, artificial Sequence
  Sequences of Human Parecho virus PSs
uucucaauuuuaggucgaugaa

SEQ ID NO: 5 - RNA, 19 nt, artificial Sequence
  Sequences of Human Parecho virus PSs
uuaaugguguuuuuacuaa

SEQ ID NO: 6 - RNA, 25 nt, artificial Sequence
  Sequences of Human Parecho virus PSs
uuaguauacuuuuguugguaacaaa

SEQ ID NO: 7 - RNA, 29 nt, artificial Sequence
  Sequences of Human Parecho virus PSs
agcugguuauaguuuuguuaaaucuggcu

SEQ ID NO: 8 - RNA, 30 nt, artificial Sequence
  Sequences of Human Parecho virus PSs
aggcuugugaaguugauuauugcauuguuu

SEQ ID NO: 9 - RNA, 23 nt, artificial Sequence
  Sequences of Human Parecho virus PSs
aagauuaauguuuuguuuuucuu

SEQ ID NO: 10 - RNA, 39 nt, artificial Sequence
  Aptamer
cgcugguucgaauuuauuaggcaagauugagaaauggcu

SEQ ID NO: 11 - RNA, 40 nt, artificial Sequence
  Aptamer
gucggucucauaagguuuuguuguucgguuuuuuguuggu

SEQ ID NO: 12 - RNA, 39 nt, artificial Sequence
  Aptamer
uucucacgauuuuugggucuuuguuuguuuguugggugg

SEQ ID NO: 13 - RNA, 27 nt, artificial Sequence
  Aptamer
auguuuuuuguuggcuuaggauuacgu

SEQ ID NO: 14 - RNA, 40 nt, artificial Sequence
  Aptamer
gucgguccguuguuaaguuguuuuuguguuuuaugguuga

SEQ ID NO: 15 - DNA, 41 bp, artificial Sequence
  Sequences of  Human immunodeficiency virus PSs
agatccctcagacccttttagtcagtgtggaaaatctctag

SEQ ID NO: 16 - DNA, 25 bp, artificial Sequence
  Sequences of  Human immunodeficiency virus PSs
aggatggatgacacataatccacct

SEQ ID NO: 17 - DNA, 34 bp, artificial Sequence
  Sequences of  Human immunodeficiency virus PSs
tagagactatgtagaccgattctataaaactcta

SEQ ID NO: 18 - DNA, 28 bp, artificial Sequence
  Sequences of  Human immunodeficiency virus PSs
tctggccttcccacaagggaaggccagg

SEQ ID NO: 19 - DNA, 29 bp, artificial Sequence
  Sequences of  Human immunodeficiency virus PSs
ttggggaagagacaacaactccctctcag

SEQ ID NO: 20 - DNA, 27 bp, artificial Sequence
  Sequences of  Human immunodeficiency virus PSs
cccctcgtcacaataaagatagggggg

SEQ ID NO: 21 - DNA, 27 bp, artificial Sequence
  Sequences of  Human immunodeficiency virus PSs
atggcccaaaagttaaacaatggccat

SEQ ID NO: 22 - DNA, 16 bp, artificial Sequence
  Sequences of  Human immunodeficiency virus PSs
tgggcctgaaaatcca

SEQ ID NO: 23 - DNA, 43 bp, artificial Sequence
  Sequences of  Human immunodeficiency virus PSs
ctccagtatttgccataaagaaaaaagacagtactaaatggag

SEQ ID NO: 24 - DNA, 21 bp, artificial Sequence
  Sequences of  Human immunodeficiency virus PSs
gagaacttaataagagaactc

SEQ ID NO: 25 - DNA, 22 bp, artificial Sequence
  Sequences of  Human immunodeficiency virus PSs
ggatgggttatgaactccatcc

SEQ ID NO: 26 - DNA, 25 bp, artificial Sequence
  Sequences of  Human immunodeficiency virus PSs
ggacagtacagcctatagtgctgcc

SEQ ID NO: 27 - DNA, 26 bp, artificial Sequence
  Sequences of  Human immunodeficiency virus PSs
agtcatctatcaatacatggatgatt

SEQ ID NO: 28 - DNA, 23 bp, artificial Sequence
  Sequences of  Human immunodeficiency virus PSs
gggagtttgtcaatacccctccc

SEQ ID NO: 29 - DNA, 51 bp, artificial Sequence
  Sequences of  Human immunodeficiency virus PSs
tggctagtgattttaacctaccacctgtagtagcaaaagaaatagtagcca

SEQ ID NO: 30 - DNA, 24 bp, artificial Sequence
  Sequences of  Human immunodeficiency virus PSs
cttcctcttaaaattagcaggaag

SEQ ID NO: 31 - DNA, 33 bp, artificial Sequence
  Sequences of  Human immunodeficiency virus PSs
acacatggaaaagattagtaaaacaccatatgt

SEQ ID NO: 32 - DNA, 39 bp, artificial Sequence
  Sequences of  Human immunodeficiency virus PSs
ggactggttttatagacatcactatgaaagtactaatcc

SEQ ID NO: 33 - DNA, 32 bp, artificial Sequence
  Sequences of  Human immunodeficiency virus PSs
caacatatctatgaaacttacggggatacttg

SEQ ID NO: 34 - DNA, 41 bp, artificial Sequence
  Sequences of  Human immunodeficiency virus PSs
ctgctgtttatccatttcagaattgggtgtcgacatagcag

SEQ ID NO: 35 - DNA, 21 bp, artificial Sequence
  Sequences of  Human immunodeficiency virus PSs
gccttaggcatctcctatggc

SEQ ID NO: 36 - DNA, 52 bp, artificial Sequence
  Sequences of  Human immunodeficiency virus PSs
ggagacagcgacgaagagctcatcagaacagtcagactcatcaagcttctct

SEQ ID NO: 37 - DNA, 21 bp, artificial Sequence
  Sequences of  Human immunodeficiency virus PSs
ggtgcagaaagaatatgcatt

SEQ ID NO: 38 - DNA, 47 bp, artificial Sequence
  Sequences of  Human immunodeficiency virus PSs
tgttacaataggaaaaataggaaatatgagacaagcacattgtaaca

SEQ ID NO: 39 - DNA, 23 bp, artificial Sequence
  Sequences of  Human immunodeficiency virus PSs
tgtacaaatgtcagcacagtaca

SEQ ID NO: 40 - DNA, 63 bp, artificial Sequence
  Sequences of  Human immunodeficiency virus PSs
tgctgttaaatggcagtctagcagaagaagatgtagtaattagatctgccaatttcacagaca

SEQ ID NO: 41 - DNA, 17 bp, artificial Sequence
  Sequences of  Human immunodeficiency virus PSs
aattgtaacgcacagtt

SEQ ID NO: 42 - DNA, 34 bp, artificial Sequence
  Sequences of  Human immunodeficiency virus PSs
ctgaaggaagtgacacaatcacactcccatgcag

SEQ ID NO: 43 - DNA, 21 bp, artificial Sequence
  Sequences of  Human immunodeficiency virus PSs
gtggacaaattagatgttcat

SEQ ID NO: 44 - DNA, 27 bp, artificial Sequence
  Sequences of  Human immunodeficiency virus PSs
gaggcgcaacagcatctgttgcaactc

SEQ ID NO: 45 - DNA, 26 bp, artificial Sequence
  Sequences of  Human immunodeficiency virus PSs
gtctggggcatcaaacagctccaggc

SEQ ID NO: 46 - DNA, 25 bp, artificial Sequence
  Sequences of  Human immunodeficiency virus PSs
ctcttcagctaccaccgcttgagag

SEQ ID NO: 47 - DNA, 51 bp, artificial Sequence
  Sequences of  Human immunodeficiency virus PSs
agcaatcacaagtagcaatacagcagctaacaatgctgcttgtgcctggct

SEQ ID NO: 48 - DNA, 15 bp, artificial Sequence
  Sequences of  Human immunodeficiency virus PSs
ggtacctttaagacc

SEQ ID NO: 49 - DNA, 20 bp, artificial Sequence
  Sequences of  Human immunodeficiency virus PSs
ggcagctgtagatcttagcc

SEQ ID NO: 50 - DNA, 29 bp, artificial Sequence
  Sequences of  Human immunodeficiency virus PSs
ccaggggtcagatatccactgacctttgg

SEQ ID NO: 51 - DNA, 21 bp, artificial Sequence
  Sequences of  Human immunodeficiency virus PSs
tggtgctacaagctagtacca

SEQ ID NO: 52 - DNA, 16 bp, artificial Sequence
  Sequences of  Human immunodeficiency virus PSs
gctgcatataagcagc

SEQ ID NO: 53 - DNA, 30 bp, artificial Sequence
  Sequences of  Human immunodeficiency virus PSs
aagcctcaataaagcttgccttgagtgctt

SEQ ID NO: 54 - RNA, 39 nt, artificial Sequence
  Sequences of  Turnip Crinkle virus PS
gggacguauaguaauagaggucagauagguaguagucuc

SEQ ID NO: 55 - RNA, 36 nt, artificial Sequence
  Sequences of  Turnip Crinkle virus PS
uagguugguaggaacggaagaggaagccacauccug

SEQ ID NO: 56 - RNA, 40 nt, artificial Sequence
  Sequences of  Turnip Crinkle virus PS
cuugcgggagcuggucgggagggagacucaaaucuccagg

SEQ ID NO: 57 - RNA, 36 nt, artificial Sequence
  Sequences of  Turnip Crinkle virus PS
acucaacaauuugagaagaggguugauggaaagagu

SEQ ID NO: 58 - RNA, 27 nt, artificial Sequence
  Sequences of  Turnip Crinkle virus PS
gucguucuacaagggcaggagggccac

SEQ ID NO: 59 - RNA, 31 nt, artificial Sequence
  Sequences of  Turnip Crinkle virus PS
ggacuacaagaagaagaugcaagauguuucc

SEQ ID NO: 60 - RNA, 28 nt, artificial Sequence
  Sequences of  Turnip Crinkle virus PS
gggaugaggggcagcaaagacguguccc

SEQ ID NO: 61 - RNA, 44 nt, artificial Sequence
  Sequences of  Turnip Crinkle virus PS
gacgcaacaggaaaacggaagaaaggcggagagaaaagugcgaa

SEQ ID NO: 62 - RNA, 48 nt, artificial Sequence
  Sequences of  Turnip Crinkle virus PS
gcucuguuuuaaacaagaaaagaaaugaagguucugcuagucacgggg

SEQ ID NO: 63 - RNA, 45 nt, artificial Sequence
  Sequences of  Turnip Crinkle virus PS
agauugggcaguucgcagguguuaaggacggacccaggcugguuu

SEQ ID NO: 64 - RNA, 41 nt, artificial Sequence
  Sequences of  Turnip Crinkle virus PS
ucaugguccaagaccaaggggacagcugggugggagcacga

SEQ ID NO: 65 - RNA, 40 nt, artificial Sequence
  Sequences of  Turnip Crinkle virus PS
guguccaaugggcaggagugaagguagcagaaaggggaca

SEQ ID NO: 66 - RNA, 37 nt, artificial Sequence
  Sequences of  Turnip Crinkle virus PS
cugaggagcagccaaaggguaaauugcaagcacucag

SEQ ID NO: 67 - RNA, 49 nt, artificial Sequence
  Aptamer
ggcaaacgguaaggccaaaagggacgaggguagagauugauagaaagcc

SEQ ID NO: 68 - RNA, 45 nt, artificial Sequence
  Aptamer
gcaacuaggaaaagggaagggcaagggaagggaccgaagagcagc

SEQ ID NO: 69 - RNA, 44 nt, artificial Sequence
  Aptamer
ggcaacuaacaagagggaggagagggaggaacguuaggguagcc

SEQ ID NO: 70 - DNA, 163 bp, artificial Sequence
  Sequences containing packaging signals of Cowpea Chlorotic Mottle virus 1 PSs
gtaatccacgagaacgaggttcaatcccttgtcgactcacggagtatcgaacttttcttaattttatttaatggcaagttctttagatcttttgaaattgatttctgagagaggcgctgacagccgaggcgcttcggacatagttgaacaacaagctgtaaag

SEQ ID NO: 71 - DNA, 201 bp, artificial Sequence
  Sequences containing packaging signals of Cowpea Chlorotic Mottle virus 1 PSs
atggaggagcttttgatttgaacttaactcaacaatataatgctccccatagtttggctggagctctgcgaatagcggagcattatgactgtctttcaagcttcccccctcttgatcccatcattgattttggtggttcttggtggcatcattattccaggaaggacacacgtattcacagttgttgtcccgtgttgggcg

SEQ ID NO: 72 - DNA, 201 bp, artificial Sequence
  Sequences containing packaging signals of Cowpea Chlorotic Mottle virus 1 PSs
atagtttggctggagctctgcgaatagcggagcattatgactgtctttcaagcttcccccctcttgatcccatcattgattttggtggttcttggtggcatcattattccaggaaggacacacgtattcacagttgttgtcccgtgttgggcgtcagagatgctgctcgacatgaagaacgactatgtagaatgcgtaagt

SEQ ID NO: 73 - DNA, 201 bp, artificial Sequence
  Sequences containing packaging signals of Cowpea Chlorotic Mottle virus 1 PSs
cgaaggcgttttacctttgttgaagtgccgttggatgaagtctgggaaaggtaaatctgaggtcattaaatttgatttcatgaatgagagcacactttcttatattcattcttggaccaatcttggttcatttttgactgagtctgtgcatgtgataggaggtactacttatctcctagaacgtgagctcttaaaatgcaa

SEQ ID NO: 74 - DNA, 201 bp, artificial Sequence
  Sequences containing packaging signals of Cowpea Chlorotic Mottle virus 1 PSs
ttggatgaagtctgggaaaggtaaatctgaggtcattaaatttgatttcatgaatgagagcacactttcttatattcattcttggaccaatcttggttcatttttgactgagtctgtgcatgtgataggaggtactacttatctcctagaacgtgagctcttaaaatgcaatattatgacctataaaatcgttgccacaaa

SEQ ID NO: 75 - DNA, 201 bp, artificial Sequence
  Sequences containing packaging signals of Cowpea Chlorotic Mottle virus 1 PSs
aacgtgagctcttaaaatgcaatattatgacctataaaatcgttgccacaaatctgaagtgtcctaaggaaacgttgcgacattgtgtttggtttgagaatatttcccaatatgtcgccgttaacattcctgaagactggaatctgactcattggaaacccgtacgtgtggcaaaaaccaccgtaagagaggttgaagaga

SEQ ID NO: 76 - DNA, 201 bp, artificial Sequence
  Sequences containing packaging signals of Cowpea Chlorotic Mottle virus 1 PSs
aatatgtcgccgttaacattcctgaagactggaatctgactcattggaaacccgtacgtgtggcaaaaaccaccgtaagagaggttgaagagattgcttttcgatgttttaaggagaataaagagtggacggagaatatgaaagcgatagcatctattctgtccgctaaatcttctacagtcattatcaacggtcaagcta

SEQ ID NO: 77 - DNA, 201 bp, artificial Sequence
  Sequences containing packaging signals of Cowpea Chlorotic Mottle virus 1 PSs
gctatcatggccggagagaggctgaacattgatgagtatcatctcgtcgcctttgctctcactatgaatttgtatcagaaatatgaaaatattcggaatttttatagtgagatggaatggaagggctgggtcaaccactttaaaactagattttggtggggaggaagtacggctacctcaagcactggtaagattcgagag

SEQ ID NO: 78 - DNA, 201 bp, artificial Sequence
  Sequences containing packaging signals of Cowpea Chlorotic Mottle virus 1 PSs
tacggctacctcaagcactggtaagattcgagagtttctggctggtaaattcccttggctgaggttagattcgtacaaagacagttttgtttttctgtcgaagatctctgatgtcaaagagtttgagaacgattctgttcccatctccagactgaggagtttcttcagcagtgaggacctcatggagcgcattgaattaga

SEQ ID NO: 79 - DNA, 201 bp, artificial Sequence
  Sequences containing packaging signals of Cowpea Chlorotic Mottle virus 1 PSs
aaggagcctaaaccggaagtgaccgttggagctgaaccaacaggccccgaagaggcatcgagacactttgccatcaaggaattctctgattattgtcgtcgccttgactgtaacgctgtgtcaaatcttcgtcgtttatgggccattgctggctgcgatgggaggactgcgagaaataagtcgatccttgaaacttatcat

SEQ ID NO: 80 - DNA, 201 bp, artificial Sequence
  Sequences containing packaging signals of Cowpea Chlorotic Mottle virus 1 PSs
ctacactatggtcagctgctcgctgtggctgctctctgtaagtgtcagtctgttcttgcattcggagacacggagcaaatttcttttaaatcgcgagatgcaactttccgcctgaaatatggtgatttgcagtttgacagtcgcgatattgttacggagacatggagatgtccgcaagatgttatttccgcagttcagact

SEQ ID NO: 81 - DNA, 201 bp, artificial Sequence
  Sequences containing packaging signals of Cowpea Chlorotic Mottle virus 1 PSs
tggtaagacttaaatctaccaagtgtgatctatttaaaactgaagaatattgcttggtggctttgactcgacataagattacctttgagtatctttatgttggtatgctatcaggtgatttaatatttagaagtatatcttgatcctgagtgtgattcacttacgaatcagttctaacggtttctataaaccgtagtcgtc

SEQ ID NO: 82 - DNA, 201 bp, artificial Sequence
  Sequences containing packaging signals of Cowpea Chlorotic Mottle virus 1 PSs
tttgagtatctttatgttggtatgctatcaggtgatttaatatttagaagtatatcttgatcctgagtgtgattcacttacgaatcagttctaacggtttctataaaccgtagtcgtcgttgcgacgccgaccgtcttacaagacgttcgagctgcctttgggttttactccttgaacccttcagaagaattcttcggagt

SEQ ID NO: 83 - DNA, 178 bp, artificial Sequence
  Sequences containing packaging signals of Cowpea Chlorotic Mottle virus 2 PSs
gtaatccacgagagcgaggttcaatcccttgtcgactcacgggtctccatcagttgaaaacagtttatacattttcttcttgatatttttcttctttacttccattaatatgtctaagttcattccagaaggtgagacttaccacgttccctcattccaatggatgtttgatcagact

SEQ ID NO: 84 - DNA, 201 bp, artificial Sequence
  Sequences containing packaging signals of Cowpea Chlorotic Mottle virus 2 PSs
gacggttcattcgttgatgaatctgagtgtgacgattggcggccggtagatacctctgatggtttcaccgaagcaatgtttgatgtgatgaatgagattcctggcgaggaaacaaaaaatacatgcgctttaagtcttgaagctgaatcaaggcaagctccagaaacttccgatatggtgccgtctgaatatacgttggca

SEQ ID NO: 85 - DNA, 201 bp, artificial Sequence
  Sequences containing packaging signals of Cowpea Chlorotic Mottle virus 2 PSs
aagtctgatattaaaccagttgtctcggatacgttacacctcgaacgagctgttgctgcaacaataacatttcatggtaaaggagttactagctgcttctcaccatattttacggcttgtttcgagaagttttcaaaagctttaaaatcaaggtttgtggtccccatagggaagatctcctccctggaactgaaaaatgtt

SEQ ID NO: 86 - DNA, 201 bp, artificial Sequence
  Sequences containing packaging signals of Cowpea Chlorotic Mottle virus 2 PSs
aacgagctgttgctgcaacaataacatttcatggtaaaggagttactagctgcttctcaccatattttacggcttgtttcgagaagttttcaaaagctttaaaatcaaggtttgtggtccccatagggaagatctcctccctggaactgaaaaatgttcccctctcgaataaatggtttcttgaggcggatttgagtaagt

SEQ ID NO: 87 - DNA, 201 bp, artificial Sequence
  Sequences containing packaging signals of Cowpea Chlorotic Mottle virus 2 PSs
tttcaaaagctttaaaatcaaggtttgtggtccccatagggaagatctcctccctggaactgaaaaatgttcccctctcgaataaatggtttcttgaggcggatttgagtaagtttgataaatctcagggtgagcttcatcttgagttccaaagagagatattgttgtcattgggttttccagcccctttgactaattggt

SEQ ID NO: 88 - DNA, 201 bp, artificial Sequence
  Sequences containing packaging signals of Cowpea Chlorotic Mottle virus 1 PSs
tttgagtaagtttgataaatctcagggtgagcttcatcttgagttccaaagagagatattgttgtcattgggttttccagcccctttgactaattggtggtgtgatttccatagggaatctatgctatcggatcctcatgctggagttaacatgccagtttcctttcagcgtcgtactggtgatgcttttacttattttgg

SEQ ID NO: 89 - DNA, 201 bp, artificial Sequence
  Sequences containing packaging signals of Cowpea Chlorotic Mottle virus 2 PSs
cattgggttttccagcccctttgactaattggtggtgtgatttccatagggaatctatgctatcggatcctcatgctggagttaacatgccagtttcctttcagcgtcgtactggtgatgcttttacttattttgggaatactttggtgactatggccatgatggcctattgttgcgatatgaacaccgtggactgtgcta

SEQ ID NO: 90 - DNA, 201 bp, artificial Sequence
  Sequences containing packaging signals of Cowpea Chlorotic Mottle virus 2 PSs
ccatagggaatctatgctatcggatcctcatgctggagttaacatgccagtttcctttcagcgtcgtactggtgatgcttttacttattttgggaatactttggtgactatggccatgatggcctattgttgcgatatgaacaccgtggactgtgctatcttttccggtgatgattctctgttaatttgtaaaagtaaacc

SEQ ID NO: 91 - DNA, 201 bp, artificial Sequence
  Sequences containing packaging signals of Cowpea Chlorotic Mottle virus 2 PSs
ggaatactttggtgactatggccatgatggcctattgttgcgatatgaacaccgtggactgtgctatcttttccggtgatgattctctgttaatttgtaaaagtaaaccacatctggatgctaatgtttttcaatctctgtttaatatggaaattaaagttatggacccaagtttgccatacgtttgtagtaagtttcttt

SEQ ID NO: 92 - DNA, 201 bp, artificial Sequence
  Sequences containing packaging signals of Cowpea Chlorotic Mottle virus 2 PSs
tattgttgcgatatgaacaccgtggactgtgctatcttttccggtgatgattctctgttaatttgtaaaagtaaaccacatctggatgctaatgtttttcaatctctgtttaatatggaaattaaagttatggacccaagtttgccatacgtttgtagtaagtttcttttagaaactgaaatgaataacttggtgtctgtg

SEQ ID NO: 93 - DNA, 201 bp, artificial Sequence
  Sequences containing packaging signals of Cowpea Chlorotic Mottle virus 2 PSs
cacatctggatgctaatgtttttcaatctctgtttaatatggaaattaaagttatggacccaagtttgccatacgtttgtagtaagtttcttttagaaactgaaatgaataacttggtgtctgtgcctgatcctatgagagagatacagagactggctaagcgaaagatcatcaaatcgcctgagttgttaagagcccact

SEQ ID NO: 94 - DNA, 201 bp, artificial Sequence
  Sequences containing packaging signals of Cowpea Chlorotic Mottle virus 2 PSs
ttattatgcaagtttgtggctctcaagtataaaaaacctgacgttgaaaacgatgtcagagtagccattgctgctttcggctactactcagaaaatttcttgagattttgcgaatgttatgcgactgaaggggtcaatatatataaggtaaaacatcccatcacccaggagtggttcgaggcctctagggatcgagacggt

SEQ ID NO: 95 - DNA, 201 bp, artificial Sequence
  Sequences containing packaging signals of Cowpea Chlorotic Mottle virus 2 PSs
cttccttgaaacttgcctatgatcgtaggagtcttagtaaggataaagaaaccgttgcgtgggtgcgtaagaccctttctaaataatgttggtcacatttaagacttgtttagtccacattaggactggttctaacagtttctttaaactgtaatcgtcgttgcgacgttggtttgcttacaagcaatcaagctgcctttg

SEQ ID NO: 96 - DNA, 201 bp, artificial Sequence
  Sequences containing packaging signals of Cowpea Chlorotic Mottle virus 2 PSs
taaagaaaccgttgcgtgggtgcgtaagaccctttctaaataatgttggtcacatttaagacttgtttagtccacattaggactggttctaacagtttctttaaactgtaatcgtcgttgcgacgttggtttgcttacaagcaatcaagctgcctttgagttttactccttgaactcttcagaagaattcttcggaattcg

SEQ ID NO: 97 - DNA, 199 bp, artificial Sequence
  Sequences containing packaging signals of Cowpea Chlorotic Mottle virus 2 PSs
ctggttctaacagtttctttaaactgtaatcgtcgttgcgacgttggtttgcttacaagcaatcaagctgcctttgagttttactccttgaactcttcagaagaattcttcggaattcgtaccagtatctcacatagtgaggtaataagactggtgggcagcgcctagtcgaaagactaggtgatctctaaggagacca

SEQ ID NO: 98 - DNA, 201 bp, artificial Sequence
  Sequences containing packaging signals of Cowpea Chlorotic Mottle virus 3 PSs
taacgctaaaccgtaccatagtaggctgttacctgactcgaactcaggcggacgtcagctgacattcacggaatagttcgatatcataattcctcgttctttgctgttatagctcccgatgtctaacactacttttagaccttttactggttcctccaggaccgtggtcgagggagaacaagccggcgcccaggatgatat

SEQ ID NO: 99 - DNA, 201 bp, artificial Sequence
  Sequences containing packaging signals of Cowpea Chlorotic Mottle virus 3 PSs
gtctaacactacttttagaccttttactggttcctccaggaccgtggtcgagggagaacaagccggcgcccaggatgatatgtcgttgttacagtcacttttttccgacaaatccagggaggagtttgctaaggagtgtaagttgggtatgtataccaatttatcctctaataaccggcttaattatatagatctagtccc

SEQ ID NO: 100 - DNA, 201 bp, artificial Sequence
  Sequences containing packaging signals of Cowpea Chlorotic Mottle virus 3 PSs
acactggtagtagagctctgaacttatttaagtcagagtatgaaaaaggtcacattccctccagcggtgtgcttagtatacctagagtgctggtttttcttgtgaggacgacaacagtgactgaatctgggagtgtcaccattagattggttgacttgataagcgcttcgtcggttgagattttagaacctgtggatggta

SEQ ID NO: 101 - DNA, 201 bp, artificial Sequence; identical to SEQ ID NO: 98
  Sequences containing packaging signals of Cowpea Chlorotic Mottle virus 3 PSs

SEQ ID NO: 102 - DNA, 201 bp, artificial Sequence
  Sequences containing packaging signals of Cowpea Chlorotic Mottle virus 3 PSs
ccagcggtgtgcttagtatacctagagtgctggtttttcttgtgaggacgacaacagtgactgaatctgggagtgtcaccattagattggttgacttgataagcgcttcgtcggttgagattttagaacctgtggatggtacgcaagaggctactattcctatttctagtcttccggctatcgtttgtttttctcctagtt

SEQ ID NO: 103 - DNA, 201 bp, artificial Sequence
  Sequences containing packaging signals of Cowpea Chlorotic Mottle virus 3 PSs
ttgacttgataagcgcttcgtcggttgagattttagaacctgtggatggtacgcaagaggctactattcctatttctagtcttccggctatcgtttgtttttctcctagttatgactgtcccatgcagatgatagggaatagacacagatgtttcggtttggtaactcaactggatggtgtcatatcctcagggtctaccg

SEQ ID NO: 104 - DNA, 201 bp, artificial Sequence
  Sequences containing packaging signals of Cowpea Chlorotic Mottle virus 3 PSs
tgatagggaatagacacagatgtttcggtttggtaactcaactggatggtgtcatatcctcagggtctaccgtcgttatgagtcatgcgtattggtctgcgaactttcgtagtaaacctaataactacaagcagtacgcacctatgtataagtatgtggaaccctttgacaggttgaaacgtttgagccgtaaacaattga

SEQ ID NO: 105 - DNA, 201 bp, artificial Sequence
  Sequences containing packaging signals of Cowpea Chlorotic Mottle virus 3 PSs
gaacccgccgaaaggacaggctgagggcgtacgattcatgtgtagctggctgggtgtgagacacaaaaaaaaaaaaaaaaaaaaaaaaaaaaaaaaaaaaaaaatctatgtttaatttgatagtaatttatcatgtctacagtcggaacagggaagttaactcgtgcacaacgaagggctgcggcccgtaagaacaagcgg

SEQ ID NO: 106 - DNA, 201 bp, artificial Sequence
  Sequences containing packaging signals of Cowpea Chlorotic Mottle virus 3 PSs
ctgccgaagctaaagtaacctcggctataactatctctctccctaatgagctatcgtccgaaaggaacaagcagctcaaggtaggtagagttttattatggcttgggttgcttcccagtgttagtggcacagtgaaatcctgtgttacagagacgcagactactgctgctgcctcctttcaggtggcattagctgtggccg

SEQ ID NO: 107 - DNA, 201 bp, artificial Sequence
  Sequences containing packaging signals of Cowpea Chlorotic Mottle virus 3 PSs
acgtttgacgactctttcactccggtgtattagtgcccgctgaagagcgttacactagtgtggcctacttgaaggctagttataaccgtttctttaaacggtaatcgttgttgaaacgtcttccttttacaagaggattgagctgcccttgggttttactccttgaacccttcggaagaactctttggagttcgtaccagt

SEQ ID NO: 108 - RNA, 39 nt, artificial Sequence
  Aptamer CCMV
gauuaugugucucuuucuaauugguuuuaacacgguuuc

SEQ ID NO: 109 - RNA, 24 nt, artificial Sequence
  Aptamer CCMV
cuguagaaauugguuuucuuucag

SEQ ID NO: 110 - RNA, 24 nt, artificial Sequence
  Aptamer CCMV
cguacguuucucuucgaaauuucg

SEQ ID NO: 111 - RNA, 27 nt, artificial Sequence
  Aptamer CCMV
ucaacgcacuuuuauuuggcaacguga

SEQ ID NO: 112 - RNA, 33 nt, artificial Sequence
  Aptamer CCMV
gcgucaacaacgguuuucucguuuuccuuacgu

SEQ ID NO: 113 - RNA, 26 nt, artificial Sequence
  Aptamer CCMV
uuucguuucgucuuccuaaauuuaaa

SEQ ID NO: 114 - DNA, 152 bp, artificial Sequence
  Sequences containing Brome Mosaic virus 1 packaging signals
gtagaccacggaacgaggttcaatcccttgtcgaccacggttctgctacttgttctttgtttttcaccaacaaaatgtcaagttctatcgatttgctgaagttgattgctgagaagggtgctgacagccagagtgcccaagacatcgtagac

SEQ ID NO: 115 - DNA, 201 bp, artificial Sequence
  Sequences containing Brome Mosaic virus 1 packaging signals
gttgttgtcctgtgttgggtgttagagacgctgcccgacatgaggagaggatgtgccgcatgcgaaaaattttgcaagaaagcgatgatttcgatgaagtcccgaacttttgtcttaaccgagctcaagattgtgatgtccaagctgattgggctatctgtatccacggcggttatgatatgggcttccaaggtctgtgtg

SEQ ID NO: 116 - DNA, 201 bp, artificial Sequence
  Sequences containing Brome Mosaic virus 1 packaging signals
ggtctgtgtgacgccatgcattcgcatggagtacgcgtactacgtggtaccgttatgttcgacggcgccatgttgtttgaccgcgagggttttcttcccttgcttaaatgtcactggcaacgtgacgggtcaggcgcggatgaggtgatcaaattcgattttgaaaatgaaagcacattatcttacatccacggatggcaa

SEQ ID NO: 117 - DNA, 201 bp, artificial Sequence
  Sequences containing Brome Mosaic virus 1 packaging signals
tatccgccaagtcgtcgactgttattattaacggtcaggctatcatggctggtgagcgcttagacattgaagattatcatctagtggcctttgctttgactttgaatctgtatcaaaagtacgaaaagcttacggccctccgcgatgggatggaatggaaaggttggtgccatcacttcaaaactaggttttggtggggtg

SEQ ID NO: 118 - DNA, 201 bp, artificial Sequence
  Sequences containing Brome Mosaic virus 1 packaging signals
ggtggagattcatccagggcgaaagtaggatggctgagaacattggctagcagatttcccctactacgtctggattcttatgcggacagttttaagtttctgactcgtctctcaaacgttgaagaatttgagcaagattctgtaccgatatcacgtttgagaacgttttggactgaagaggacttattcgaccggctggag

SEQ ID NO: 119 - DNA, 201 bp, artificial Sequence
  Sequences containing Brome Mosaic virus 1 packaging signals
tggattgatggacacataaaaacagtacacgaagcgcaagggatctctgttgacaacgtcactttggttcggcttaagtcgaccaaatgtgatttgtttaaacatgaggagtactgtttggttgccttaacacgacacaagaagtcctttgagtattgctttaacggcgagctcgctggtgatttgatctttaattgtgtt

SEQ ID NO: 120 - DNA, 201 bp, artificial Sequence
  Sequences containing Brome Mosaic virus 1 packaging signals
taaacatgaggagtactgtttggttgccttaacacgacacaagaagtcctttgagtattgctttaacggcgagctcgctggtgatttgatctttaattgtgttaagtgatgcgcttgtctctgtgtgagacctctgctcgaggagagccctgttccaggtaggaacgttgtggtctaactcaagactagctgaatcggtgc

SEQ ID NO: 121 - DNA, 201 bp, artificial Sequence
  Sequences containing Brome Mosaic virus 1 packaging signals
tcaagactagctgaatcggtgctataaccgatagtcgtggttgacacgcagacctcttacaagagtgtctaggcgcctttgagagttactctttgctctcttcggaagaacccttaggggttcgtgcatgggcttgcatagcaagtcttagaatgcgggtgtcgtacagtgttgaaaaacactgtaaatctctaaaagaga

SEQ ID NO: 122 - DNA, 187 bp, artificial Sequence
  Sequences containing Brome Mosaic virus 2 packaging signals
gtaaaccacggaacgaggttcaatcccttgtcgacccacggtttgcgcaacacacatctgaccttgttgttgttgtgtgcttgttctttctactatcaccaagatgtcttcgaaaacctgggatgatgatttcgttcgccaggtcccgtctttccaatggatcatagatcaatccttagaagacgag

SEQ ID NO: 123 - DNA, 201 bp, artificial Sequence
  Sequences containing Brome Mosaic virus 2 packaging signals
cctgttgtaactgacacccttcacttggaacgagcagtagcagctactataacatttcatagtaaaggtgtgactagtaatttttcaccctttttcactgcttgtttcgagaagttatcactggccctgaaatccaggttcattgtgcctatcggaaagatatcctctctggagcttaagaatgtccgcttgaataacaga

SEQ ID NO: 124 - DNA, 201 bp, artificial Sequence
  Sequences containing Brome Mosaic virus 2 packaging signals
cagggtgagctgcacctagagtttcagagagagatactccttgcgctgggctttccagcgccgctgacgaattggtggtctgattttcatcgcgattcttatttatcagaccctcatgccaaggtgggaatgtccgtttccttccaacgcagaactggtgacgcgtttacatatttcggtaatactcttgtcactatggct

SEQ ID NO: 125 - DNA, 201 bp, artificial Sequence
  Sequences containing Brome Mosaic virus 2 packaging signals
acatatttcggtaatactcttgtcactatggctatgattgcatatgcctctgatctaagtgactgtgactgtgcaatattttcaggagatgattctttaatcatctctaaagttaagccagtcctggataccgatatgtttacgtctctcttcaatatggagataaaagtcatggaccctagtgtgccctacgtttgtagt

SEQ ID NO: 126 - DNA, 201 bp, artificial Sequence
  Sequences containing Brome Mosaic virus 2 packaging signals
gggcaatttggtgtctgtaccagatcctctgagagagatccagcgcttagctaagcgaaagattctgcgtgatgaacagatgctcagagcacatttcgtttccttctgtgatcgaatgaagtttattaatcaacttgatgagaagatgattacgacgctctgtcattttgtttatctgaaatatgggaaagaaaaaccttg

SEQ ID NO: 127 - DNA, 201 bp, artificial Sequence
  Sequences containing Brome Mosaic virus 2 packaging signals
cgtgatgaacagatgctcagagcacatttcgtttccttctgtgatcgaatgaagtttattaatcaacttgatgagaagatgattacgacgctctgtcattttgtttatctgaaatatgggaaagaaaaaccttggattttcgaggaggttagagctgctcttgcggctttttctttatactccgagaatttcctgaggttc

SEQ ID NO: 128 - DNA, 201 bp, artificial Sequence
  Sequences containing Brome Mosaic virus 2 packaging signals
acgacgctctgtcattttgtttatctgaaatatgggaaagaaaaaccttggattttcgaggaggttagagctgctcttgcggctttttctttatactccgagaatttcctgaggttctctgattgctactgtaccgaaggcatcagagtttatcagatgagcgatcctgtatgtaagttcaaacgcaccacggaagagcgt

SEQ ID NO: 129 - DNA, 201 bp, artificial Sequence
  Sequences containing Brome Mosaic virus 2 packaging signals
taaaagcttgttgaatcagtacaataactgatagtcgtggttgacacgcagacctcttacaagagtgtctaggtgcctttgagagttactctttgctctcttcggaagaacccttaggggttcgtgcatgggcttgcatagcaagtcttagaatgcgggtgccgtacagtgttgaaaaacactgtaaatctctaaaagaga

SEQ ID NO: 130 - DNA, 168 bp, artificial Sequence
  Sequences containing Brome Mosaic virus 3 packaging signals
gtaaaataccaactaattctcgttcgattccggcgaacattctattttaccaacatcggttttttcagtagtgatactgtttttgttcccgatgtctaacatagtttctcccttcagtggttcctcacgaactacgtctgacgttggcaagcaagcgggaggtactag

SEQ ID NO: 131 - DNA, 201 bp, artificial Sequence
  Sequences containing Brome Mosaic virus 3 packaging signals
cacacgtatctgcttggctctcatgggctacatccaagtatgataaaggagagttaccttccaggggattcatgaacgttccacgcatcgtttgttttctcgttcgtaccacagatagcgcagagtccggttctataaccgtgagcctgtgcgattctggtaaggctgctcgtgctggagtactcgaagccattgataatc

SEQ ID NO: 132 - DNA, 201 bp, artificial Sequence
  Sequences containing Brome Mosaic virus 3 packaging signals
aaatccggtctaacaagctcggtccatttcgtagagttaagcaagctggggagacccccgacagccgtttggatcagcgctcgcgtctcgtttgggttcaattcccttaccttacaacggcgtgttgagataggtcctcgggggaggttatccatgtttgtggatattctatgttgtgtgtctgagttattattaaaaaaa

SEQ ID NO: 133 - DNA, 201 bp, artificial Sequence
  Sequences containing Brome Mosaic virus 3 packaging signals
gtttggatcagcgctcgcgtctcgtttgggttcaattcccttaccttacaacggcgtgttgagataggtcctcgggggaggttatccatgtttgtggatattctatgttgtgtgtctgagttattattaaaaaaaaaaaaaaaagatctatgtcctaattcagcgtattaataatgtcgacttcaggaactggtaagatga

SEQ ID NO: 134 - DNA, 201 bp, artificial Sequence
  Sequences containing Brome Mosaic virus 3 packaging signals
ggttcaattcccttaccttacaacggcgtgttgagataggtcctcgggggaggttatccatgtttgtggatattctatgttgtgtgtctgagttattattaaaaaaaaaaaaaaaagatctatgtcctaattcagcgtattaataatgtcgacttcaggaactggtaagatgactcgcgcgcagcgtcgtgctgccgctcg

SEQ ID NO: 135 - DNA, 201 bp, artificial Sequence
  Sequences containing Brome Mosaic virus 3 packaging signals
ggttaaaagcttgttgaatcagtacaataactgatagtcgtggttgacacgcagacctcttacaagagtgtctaggtgcctttgagagttactctttgctctcttcggaagaacccttaggggttcgtgcatgggcttgcatagcaagtcttagaatgcgggtaccgtacagtgttgaaaaacactgtaaatctctaaaag

SEQ ID NO: 136 - protein, 350 aa, Turnip crinkle virus
MENDPRVRKFASDGAQWAIKWQKKGWSTLTSRQKQTARAAMGIKLSPVAQPVQKVTRLSAPVALAYREVSTQPRVSTARDGITRSGSELITTLKKNTDTEPKYTTAVLNPSEPGTFNQLIKEAAQYEKYRFTSLRFRYSPMSPSTTGGKVALAFDRDAAKPPPNDLASLYNIEGCVSSVPWTGFILTVPTDSTDRFVADGISDPKLVDFGKLIMATYGQGANDAAQLGEVRVEYTVQLKNRTGSTSDAQIGQFAGVKDGPRLVSWSKTKGTAGWEHDCHFLGTGNFSLTLFYEKAPVSGLENADASDFSVLGEAAAGSVQWAGVKVAERGQGVKMVTTEEQPKGKLQALR

SEQ ID NO: 137 - protein, 770 aa, Human Parechovirus 1
METIKSIADMATGVVSSVDSTINAVNEKVESVGNEIGGNLLTKVADDASNILGPNCFATTAEPENKNVVQATTTVNTTNLTQHPSAPTMPFSPDFSNVDNFHSMAYDITTGDKNPSKLVRLETHEWTPSWARGYQITHVELPKVFWDHQDKPAYGQSRYFAAVRCGFHFQVQVNVNQGTAGSALVVYEPKPVVTYDSKLEFGAFTNLPHVLMNLAETTQADLCIPYVADTNYVKTDSSDLGQLKVYVWTPLSIPTGSANQVDVTILGSLLQLDFQNPRVFAQDVNIYDNAPNGKKKNWKKIMTMSTKYKWTRTKIDIAEGPGSMNMANVLCTTGAQSVALVGERAFYDPRTAGSKSRFDDLVKIAQLFSVMADSTTPSENHGVDAKGYFKWSATTAPQSIVHRNIVYLRLFPNLNVFVNSYSYFRGSLVLRLSVYASTFNRGRLRMGFFPNATTDSTSTLDNAIYTICDIGSDNSFEITIPYSFSTWMRKTNGHPIGLFQIEVLNRLTYNSSSPSEVYCIVQGKMGQDARFFCPTGSVVTFQNSWGSQMDLTDPLCIEDDTENCKQTMSPNELGLTSAQDDGPLGQEKPNYFLNFRSMNVDIFTVSHTKVDNLFGRAWFFMEHTFTNEGQWRVPLEFPKQGHGSLSLLFAYFTGELNIHVLFLSERGFLRVAHTYDTSNDRVNFLSSNGVITVPAGEQMTLSAPYYSNKPLRTVRDNNSLGYLMCKPFLTGTSTGKIEVYLSLRCPNFFFPLPAPKVTSSRALRGDMANL

SEQ ID NO: 138 - protein, 190 aa, Cowpea Chlorotic Mottle virus
MSTVGTGKLTRAQRRAAARKNKRNTRVVQPVIVEPIASGQGKAIKAWTGYSVSKWTASCAAAEAKVTSAITISLPNELSSERNKQLKVGRVLLWLGLLPSVSGTVKSCVTETQTTAAASFQVALAVADNSKDVVAAMYPEAFKGITLEQLAADLTIYLYSSAALTEGDVIVHLEVEHVRPTFDDSFTPVY

SEQ ID NO: 139 - protein, 189 aa, Brome Mosaic Virus
MSTSGTGKMTRAQRRAAARRNRWTARVQPVIVEPLAAGQGKAIKAIAGYSISKWEASSDAITAKATNAMSITLPHELSSEKNKELKVGRVLLWLGLLPSVAGRIKACVAEKQAQAEAAFQVALAVADSSKEVVAAMYTDAFRGATLGDLLNLQIYLYASEAVPAKAVVVHLEVEHVRPTFDDFFTPVYR

SEQ ID NO: 140 - protein, 85 aa, Human immunodeficiency virus
AEAMSQVTNPATIMIQKGNFRNQRKTVKCFNCGKEGHIAKNCRAPRKKGCWKCGKEGHQMKDCTERQANFLGKIWPSHKGRPGNF

SEQ ID NO: 141 - protein, 215 aa, Human immunodeficiency virus
PRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTHNPPIPVGEIYKRWIILGLNKIVRMYSPTSILDIRQGPKEPFRDYVDRFYKTLRAEQASQEVKNWMTETLLVQNANPDCKTILKALGPGATLEEMMTACQGVGGPGHKARVL

SEQ ID NO: 142 - RNA, 34 nt, Artificial Sequence
  Hepatitis B Virus
uuuguuuaaagacugggaggaguugggggaggag

SEQ ID NO: 143 - RNA, 53 nt, Artificial Sequence
  Hepatitis virus B
gugggcccucugacaguuaaugaaaaaaggagauuaaaauuaauuaugccugc

SEQ ID NO: 144 - RNA, 44 nt, Artificial Sequence
  Hepatitis virus B
ggaaggcuggcauucuauauaagagagaaacuacacgcagcgcc

SEQ ID NO: 145 - RNA, 34 nt, artificial sequence
  bmv1_ps
cuuguucuuuguuuuucaccaacaaaaugucaag

SEQ ID NO: 146 - RNA, 12 nt, artificial sequence
  BMV1_PS
cucucuauugag

SEQ ID NO: 147 - RNA, 20 nt, artificial sequence
  BMV1_PS
agcucucuauugaggaggcu

SEQ ID NO: 148 - RNA, 20 nt, artificial sequence
  BMV1_PS
uugacuuaaauuugacucag

SEQ ID NO: 149 - RNA, 26 nt, artificial sequence
  BMV1_PS
gucucgacaguuuucccccugaagac

SEQ ID NO: 150 - RNA, 11 nt, artificial sequence
  BMV1_PS
gcacaguuguu

SEQ ID NO: 151 - RNA, 11 nt, artificial sequence
  BMV1_PS
gcacaguuguu

SEQ ID NO: 152 - RNA, 20 nt, artificial sequence
  BMV1_PS
aagucccgaacuuuugucuu

SEQ ID NO: 153 - RNA, 10 nt, artificial sequence
  BMV1_PS
ucuuaaccga

SEQ ID NO: 154 - RNA, 29 nt, artificial sequence
  BMV1_PS
ugaccgcgaggguuuucuucccuugcuua

SEQ ID NO: 155 - RNA, 12 nt, artificial sequence
  BMV1_PS
ggguuuucuucc

SEQ ID NO: 156 - RNA, 14 nt, artificial sequence
  BMV1_PS
gaucaaauucgauu

SEQ ID NO: 157 - RNA, 15 nt, artificial sequence
  BMV1_PS
gcuacaaauuuacgc

SEQ ID NO: 158 - RNA, 21 nt, artificial sequence
  BMV1_PS
gagauagcuuucagauguuuc

SEQ ID NO: 159 - RNA, 29 nt, artificial sequence
  BMV1_PS
cuucaaaacuagguuuuggugggguggag

SEQ ID NO: 160 - RNA, 16 nt, artificial sequence
  BMV1_PS
augacguuaaaccggu

SEQ ID NO: 161 - RNA, 20 nt, artificial sequence
  BMV1_PS
gcaugguuuagguccgaagc

SEQ ID NO: 162 - RNA, 17 nt, artificial sequence
  BMV1_PS
caugcgauauuuccaug

SEQ ID NO: 163 - RNA, 10 nt, artificial sequence
  BMV1_PS
accuaauugu

SEQ ID NO: 164 - RNA, 12 nt, artificial sequence
  BMV1_PS
ggcuuuauuccc

SEQ ID NO: 165 - RNA, 14 nt, artificial sequence
  BMV1_PS
cuuauaauuccaag

SEQ ID NO: 166 - RNA, 34 nt, artificial sequence
  BMV1_PS
guugaugaggcugguuuacuacauuauggucaac

SEQ ID NO: 167 - RNA, 34 nt, artificial sequence; identical to SEQ ID NO: 166
  BMV1_PS

SEQ ID NO: 168 - RNA, 30 nt, artificial sequence
  BMV1_PS
gggacacagagcagauuucguucaagucuc

SEQ ID NO: 169 - RNA, 10 nt, artificial sequence
  BMV1_PS
gauuucguuc

SEQ ID NO: 170 - RNA, 30 nt, artificial sequence
  BMV1_PS
ucgugacgcggguuuuaaauugcuccacgg

SEQ ID NO: 171 - RNA, 16 nt, artificial sequence
  BMV1_PS
ggguuuuaaauugcuc

SEQ ID NO: 172 - RNA, 10 nt, artificial sequence
  BMV1_PS
ggauuuuccc

SEQ ID NO: 173 - RNA, 9 nt, artificial sequence
  BMV1_PS
ggauuuucc

SEQ ID NO: 174 - RNA, 25 nt, artificial sequence
  BMV1_PS
uuugguucggcuuaagucgaccaaa

SEQ ID NO: 175 - RNA, 10 nt, artificial sequence
  BMV1_PS
uguuuaaaca

SEQ ID NO: 176 - RNA, 25 nt, artificial sequence
  BMV1_PS
uuugguugccuuaacacgacacaag

SEQ ID NO: 177 - RNA, 17 nt, artificial sequence
  BMV1_PS
uuugaguauugcuuuaa

SEQ ID NO: 178 - RNA, 26 nt, artificial sequence
  BMV1_PS
ugaguauugcuuuaacggcgagcucg

SEQ ID NO: 179 - RNA, 10 nt, artificial sequence
  BMV1_PS
uguuuaaaca

SEQ ID NO: 180 - RNA, 25 nt, artificial sequence
  BMV1_PS
uuugguugccuuaacacgacacaag

SEQ ID NO: 181 - RNA, 17 nt, artificial sequence
  BMV1_PS
uuugaguauugcuuuaa

SEQ ID NO: 182 - RNA, 26 nt, artificial sequence
  BMV1_PS
ugaguauugcuuuaacggcgagcucg

SEQ ID NO: 183 - RNA, 23 nt, artificial sequence
  BMV1_PS
ugauuugaucuuuaauuguguua

SEQ ID NO: 184 - RNA, 26 nt, artificial sequence
  Hepatitis C Virus PS
cgaccucaugggguacauccccgucg

SEQ ID NO: 185 - RNA, 29 nt, artificial sequence
  Hepatitis C Virus PS
ccugacccugggggaagccaugauucagg

SEQ ID NO: 186 - RNA, 23 nt, artificial sequence
  Hepatitis C Virus PS
gggacaagcggggagcauugcuc

SEQ ID NO: 187 - RNA, 23 nt, artificial sequence
  Hepatitis C Virus PS
uaccagcucagggagauguggug

SEQ ID NO: 188 - RNA, 25 nt, artificial sequence
  Hepatitis C Virus PS
ucagcgccgcgggcgcacagguaga

SEQ ID NO: 189 - RNA, 20 nt, artificial sequence
  Hepatitis C Virus PS
uaggccuggguaaggugcug

SEQ ID NO: 190 - RNA, 30 nt, artificial sequence
  Hepatitis C Virus PS
cgugggaccgggggagggcgcgguccaaug

SEQ ID NO: 191 - RNA, 19 nt, artificial sequence
  Hepatitis C Virus PS
cccccccaggggggggggg

SEQ ID NO: 192 - RNA, 27 nt, artificial sequence
  bmv2_packaging signal
ggaacgagguucaaucccuugucgacc

SEQ ID NO: 193 - RNA, 10 nt, artificial sequence
  bmv2_packaging signal
gguucaaucc

SEQ ID NO: 194 - RNA, 23 nt, artificial sequence
  bmv2_packaging signal
gugcuuguucuuucuacuaucac

SEQ ID NO: 195 - RNA, 27 nt, artificial sequence
  bmv2_packaging signal
ccugggaugaugauuucguucgccagg

SEQ ID NO: 196 - RNA, 18 nt, artificial sequence
  bmv2_packaging signal
ugaugauuucguucgcca

SEQ ID NO: 197 - RNA, 15 nt, artificial sequence
  bmv2_packaging signal
ccgucuuuccaaugg

SEQ ID NO: 198 - RNA, 20 nt, artificial sequence
  bmv2_packaging signal
cugcuagccuucaggugcag

SEQ ID NO: 199 - RNA, 18 nt, artificial sequence
  bmv2_packaging signal
cagacggaguugccauug

SEQ ID NO: 200 - RNA, 12 nt, artificial sequence
  bmv2_packaging signal
guugccauugac

SEQ ID NO: 201 - RNA, 25 nt, artificial sequence
  bmv2_packaging signal
cgcgaguuuuaaauuagcuauagcg

SEQ ID NO: 202 - RNA, 16 nt, artificial sequence
  bmv2_packaging signal
gggguauucgaucccc

SEQ ID NO: 203 - RNA, 23 nt, artificial sequence
  bmv2_packaging signal
gguauucgaucccccuuuugacc

SEQ ID NO: 204 - RNA, 28 nt, artificial sequence
  bmv2_packaging signal
guauucgaucccccuuuugaccgagugc

SEQ ID NO: 205 - RNA, 19 nt, artificial sequence
  bmv2_packaging signal
uggggcucuauuugcgaca

SEQ ID NO: 206 - RNA, 23 nt, artificial sequence
  bmv2_packaging signal
gggcucuauuugcgacaccgucc

SEQ ID NO: 207 - RNA, 35 nt, artificial sequence
  bmv2_packaging signal
aucuugacauuccgggcucuuucgugcucgaagau

SEQ ID NO: 208 - RNA, 14 nt, artificial sequence
  bmv2_packaging signal
caugggcauugaug

SEQ ID NO: 209 - RNA, 29 nt, artificial sequence
  bmv2_packaging signal
gguuucgcguguuauugauacacacugcc

SEQ ID NO: 210 - RNA, 29 nt, artificial sequence
  bmv2_packaging signal
ucucuacugggccaauuuauauggagaga

SEQ ID NO: 211 - RNA, 36 nt, artificial sequence
  bmv2_packaging signal
aagcgaccagucauuccauacugccaacccaugcuu

SEQ ID NO: 212 - RNA, 28 nt, artificial sequence
  bmv2_packaging signal
uaccaucaagcccuuguugaaaauggug

SEQ ID NO: 213 - RNA, 28 nt, artificial sequence
  bmv2_packaging signal
uaccaucaagcccuuguugaaaauggug

SEQ ID NO: 214 - RNA, 33 nt, artificial sequence
  bmv2_packaging signal
guugaaaauggugauuauuccauggacuuugau

SEQ ID NO: 215 - RNA, 14 nt, artificial sequence
  bmv2_packaging signal
acauuccuuaaugu

SEQ ID NO: 216 - RNA, 21 nt, artificial sequence
  bmv2_packaging signal
gcacauggacuugcaaggugu

SEQ ID NO: 217 - RNA, 14 nt, artificial sequence
  bmv2_packaging signal
gacugauuuauguc

SEQ ID NO: 218 - RNA, 12 nt, artificial sequence
  bmv2_packaging signal
cccuucacuugg

SEQ ID NO: 219 - RNA, 29 nt, artificial sequence
  bmv2_packaging signal
acugacacccuucacuuggaacgagcagu

SEQ ID NO: 220 - RNA, 29 nt, artificial sequence
  bmv2_packaging signal
acugacacccuucacuuggaacgagcagu

SEQ ID NO: 221 - RNA, 29 nt, artificial sequence
  bmv2_packaging signal
acugacacccuucacuuggaacgagcagu

SEQ ID NO: 222 - RNA, 24 nt, artificial sequence
  bmv2_packaging signal
cuacuauaacauuucauaguaaag

SEQ ID NO: 223 - RNA, 18 nt, artificial sequence
  bmv2_packaging signal
guuucgagaaguuaucac

SEQ ID NO: 224 - RNA, 24 nt, artificial sequence
  bmv2_packaging signal
uccagguucauugugccuaucgga

SEQ ID NO: 225 - RNA, 17 nt, artificial sequence
  bmv2_packaging signal
ggagcuuaagaaugucc

SEQ ID NO: 226 - RNA, 18 nt, artificial sequence
  bmv2_packaging signal
aauaacagauacuuucuu

SEQ ID NO: 227 - RNA, 14 nt, artificial sequence
  bmv2_packaging signal
ggcuuuccagcgcc

SEQ ID NO: 228 - RNA, 30 nt, artificial sequence
  bmv2_packaging signal
gaauugguggucugauuuucaucgcgauuc

SEQ ID NO: 229 - RNA, 26 nt, artificial sequence
  bmv2_packaging signal
gguggucugauuuucaucgcgauucu

SEQ ID NO: 230 - RNA, 14 nt, artificial sequence
  bmv2_packaging signal
gauucuuauuuauc

SEQ ID NO: 231 - RNA, 21 nt, artificial sequence
  bmv2_packaging signal
uccguuuccuuccaacgcaga

SEQ ID NO: 232 - RNA, 21 nt, artificial sequence
  bmv2_packaging signal
guuuacauauuucgguaauac

SEQ ID NO: 233 - RNA, 8 nt, artificial sequence
  bmv2_packaging signal
acucuugu

SEQ ID NO: 234 - RNA, 11 nt, artificial sequence
  bmv2_packaging signal
gcuaugauugc

SEQ ID NO: 235 - RNA, 30 nt, artificial sequence
  bmv2_packaging signal
uggauaccgauauguuuacgucucucuuca

SEQ ID NO: 236 - RNA, 16 nt, artificial sequence
  bmv2_packaging signal
gauauguuuacgucuc

SEQ ID NO: 237 - RNA, 21 nt, artificial sequence
  bmv2_packaging signal
gucucucuucaauauggagau

SEQ ID NO: 238 - RNA, 14 nt, artificial sequence
  bmv2_packaging signal
gcgaaagauucugc

SEQ ID NO: 239 - RNA, 34 nt, artificial sequence
  bmv2_packaging signal
acagaugcucagagcacauuucguuuccuucugu

SEQ ID NO: 240 - RNA, 24 nt, artificial sequence
  bmv2_packaging signal
augaaguuuauuaaucaacuugau

SEQ ID NO: 241 - RNA, 11 nt, artificial sequence
  bmv2_packaging signal
aucaacuugau

SEQ ID NO: 242 - RNA, 29 nt, artificial sequence
  bmv2_packaging signal
ucugucauuuuguuuaucugaaauauggg

SEQ ID NO: 243 - RNA, 27 nt, artificial sequence
  bmv2_packaging signal
cugucauuuuguuuaucugaaauaugg

SEQ ID NO: 244 - RNA, 18 nt, artificial sequence
  bmv2_packaging signal
gaaaaaccuuggauuuuc

SEQ ID NO: 245 - RNA, 28 nt, artificial sequence
  bmv2_packaging signal
gguuagagcugcucuugcggcuuuuucu

SEQ ID NO: 246 - RNA, 18 nt, artificial sequence
  bmv2_packaging signal
cuccgagaauuuccugag

SEQ ID NO: 247 - RNA, 18 nt, artificial sequence
  bmv2_packaging signal
cuccgagaauuuccugag

SEQ ID NO: 248 - RNA, 19 nt, artificial sequence
  bmv2_packaging signal
caucagaguuuaucagaug

SEQ ID NO: 249 - RNA, 18 nt, artificial sequence
  bmv2_packaging signal
uguauguaaguucaaacg

SEQ ID NO: 250 - RNA, 20 nt, artificial sequence
  bmv2_packaging signal
guauguaaguucaaacgcac

SEQ ID NO: 251 - RNA, 32 nt, artificial sequence
  bmv2_packaging signal
cuggaagaauccaaaguuuccugguguguuag

SEQ ID NO: 252 - RNA, 21 nt, artificial sequence
  bmv2_packaging signal
ccauuggaauuuauuccucgg

SEQ ID NO: 253 - RNA, 33 nt, artificial sequence
  bmv2_packaging signal
guagaggaggccuaacgucaguugaugcuuugc

SEQ ID NO: 254 - RNA, 17 nt, artificial sequence
  bmv2_packaging signal
gagacuuuuaagcccuc

SEQ ID NO: 255 - RNA, 22 nt, artificial sequence
  bmv2_packaging signal
gccuuugagaguuacucuuugc

SEQ ID NO: 256 - RNA, 32 nt, artificial sequence
  bmv2_packaging signal
cuuugagaguuacucuuugcucucuucggaag

SEQ ID NO: 257 - RNA, 17 nt, artificial sequence
  bmv3_packaging signal
guucgauuccggcgaac

SEQ ID NO: 258 - RNA, 11 nt, artificial sequence
  bmv3_packaging signal
aguuucucccu

SEQ ID NO: 259 - RNA, 33 nt, artificial sequence
  bmv3_packaging signal
uaguuucucccuucagugguuccucacgaacua

SEQ ID NO: 260 - RNA, 15 nt, artificial sequence
  bmv3_packaging signal
aaggagaguuaccuu

SEQ ID NO: 261 - RNA, 14 nt, artificial sequence
  bmv3_packaging signal
ggagaguuaccuuc

SEQ ID NO: 262 - RNA, 15 nt, artificial sequence
  bmv3_packaging signal
ggagaguuaccuucc

SEQ ID NO: 263 - RNA, 15 nt, artificial sequence
  bmv3_packaging signal
ggagaguuaccuucc

SEQ ID NO: 264 - RNA, 21 nt, artificial sequence
  bmv3_packaging signal
gaguuaccuuccaggggauuc

SEQ ID NO: 265 - RNA, 20 nt, artificial sequence
  bmv3_packaging signal
augaacguuccacgcaucgu

SEQ ID NO: 266 - RNA, 18 nt, artificial sequence
  bmv3_packaging signal
cguuuguuuucucguucg

SEQ ID NO: 267 - RNA, 18 nt, artificial sequence
  bmv3_packaging signal
ggccacaauucaguuguc

SEQ ID NO: 268 - RNA, 9 nt, artificial sequence
  bmv3_packaging signal
ggcuuuacc

SEQ ID NO: 269 - RNA, 29 nt, artificial sequence
  bmv3_packaging signal
aguugucggcuuuaccugcuuugauagcu

SEQ ID NO: 270 - RNA, 26 nt, artificial sequence
  bmv3_packaging signal
ccguugcaguuacucaugcguauugg

SEQ ID NO: 271 - RNA, 29 nt, artificial sequence
  bmv3_packaging signal
aguuacucaugcguauuggcaagcuaauu

SEQ ID NO: 272 - RNA, 25 nt, artificial sequence
  bmv3_packaging signal
ggcaagcuaauuucaaagcgaagcc

SEQ ID NO: 273 - RNA, 30 nt, artificial sequence
  bmv3_packaging signal
auggucccgcuacaauuaugguaaugccau

SEQ ID NO: 274 - RNA, 23 nt, artificial sequence
  bmv3_packaging signal
gccucaaaaauuauauuagaggu

SEQ ID NO: 275 - RNA, 23 nt, artificial sequence
  bmv3_packaging signal
gccucaaaaauuauauuagaggu

SEQ ID NO: 276 - RNA, 10 nt, artificial sequence
  bmv3_packaging signal
agguauuucu

SEQ ID NO: 277 - RNA, 23 nt, artificial sequence
  bmv3_packaging signal
uagagguauuucuaaccagucug

SEQ ID NO: 278 - RNA, 20 nt, artificial sequence
  bmv3_packaging signal
gauuuguuaguugaggaauc

SEQ ID NO: 279 - RNA, 16 nt, artificial sequence
  bmv3_packaging signal
gagucuccuuccgcuc

SEQ ID NO: 280 - RNA, 38 nt, artificial sequence
  bmv3_packaging signal
cgucaucugucgcuggacuuccuguguccaguccuacg

SEQ ID NO: 281 - RNA, 18 nt, artificial sequence
  bmv3_packaging signal
gcuuagaauuaaauaggu

SEQ ID NO: 282 - RNA, 12 nt, artificial sequence
  bmv3_packaging signal
guagaguuaagc

SEQ ID NO: 283 - RNA, 21 nt, artificial sequence
  bmv3_packaging signal
guuuggguucaauucccuuac

SEQ ID NO: 284 - RNA, 11 nt, artificial sequence
  bmv3_packaging signal
ccuuacaacgg

SEQ ID NO: 285 - RNA, 26 nt, artificial sequence
  bmv3_packaging signal
cauguuuguggauauucuauguugug

SEQ ID NO: 286 - RNA, 21 nt, artificial sequence
  bmv3_packaging signal
ucagcguauuaauaaugucga

SEQ ID NO: 287 - RNA, 22 nt, artificial sequence
  bmv3_packaging signal
gcaaggccauuaaagcgauugc

SEQ ID NO: 288 - RNA, 13 nt, artificial sequence
  bmv3_packaging signal
cgcgauuacagcg

SEQ ID NO: 289 - RNA, 14 nt, artificial sequence
  bmv3_packaging signal
gcuuuucaaguagc

SEQ ID NO: 290 - RNA, 12 nt, artificial sequence
  bmv3_packaging signal
cagauuuaucug

SEQ ID NO: 291 - RNA, 23 nt, artificial sequence
  bmv3_packaging signal
uguacaucuagaaguugagcacg

SEQ ID NO: 292 - RNA, 21 nt, artificial sequence
  bmv3_packaging signal
caccccgguuuauagguagug

SEQ ID NO: 293 - RNA, 27 nt, artificial sequence
  bmv3_packaging signal
gccccugacuggguuaaagucacaggc

SEQ ID NO: 294 - RNA, 19 nt, artificial sequence
  bmv3_packaging signal
gcuaagguuaaaagcuugu

SEQ ID NO: 295 - RNA, 22 nt, artificial sequence
  bmv3_packaging signal
gccuuugagaguuacucuuugc

SEQ ID NO: 296 - RNA, 29 nt, artificial sequence
  CCMV1_packaging signal
gagaacgagguucaaucccuugucgacuc

SEQ ID NO: 297 - RNA, 19 nt, artificial sequence
  CCMV1_packaging signal
ucuuaauuuuauuuaaugg

SEQ ID NO: 298 - RNA, 8 nt, artificial sequence
  CCMV1_packaging signal
ucuuuuga

SEQ ID NO: 299 - RNA, 28 nt, artificial sequence
  CCMV1_packaging signal
uuuagaucuuuugaaauugauuucugag

SEQ ID NO: 300 - RNA, 25 nt, artificial sequence
  CCMV1_packaging signal
ucuuuugaaauugauuucugagaga

SEQ ID NO: 301 - RNA, 25 nt, artificial sequence
  CCMV1_packaging signal
ucuuuugaaauugauuucugagaga

SEQ ID NO: 302 - RNA, 22 nt, artificial sequence
  CCMV1_packaging signal
gcuguaaagcaauugcuugagc

SEQ ID NO: 303 - RNA, 19 nt, artificial sequence
  CCMV1_packaging signal
caauugcuugagcaaguug

SEQ ID NO: 304 - RNA, 23 nt, artificial sequence
  CCMV1_packaging signal
uugauuugaacuuaacucaacaa

SEQ ID NO: 305 - RNA, 23 nt, artificial sequence
  CCMV1_packaging signal
gcuccccauaguuuggcuggagc

SEQ ID NO: 306 - RNA, 11 nt, artificial sequence
  CCMV1_packaging signal
uaguuuggcug

SEQ ID NO: 307 - RNA, 12 nt, artificial sequence
  CCMV1_packaging signal
cugucuuucaag

SEQ ID NO: 308 - RNA, 26 nt, artificial sequence
  CCMV1_packaging signal
gaucccaucauugauuuuggugguuc

SEQ ID NO: 309 - RNA, 28 nt, artificial sequence
  CCMV1_packaging signal
acacguauucacaguuguugucccgugu

SEQ ID NO: 310 - RNA, 24 nt, artificial sequence
  CCMV1_packaging signal
uuguugucccguguugggcgucag

SEQ ID NO: 311 - RNA, 21 nt, artificial sequence
  CCMV1_packaging signal
uugucccguguugggcgucag

SEQ ID NO: 312 - RNA, 18 nt, artificial sequence
  CCMV1_packaging signal
gccauauguauucauggu

SEQ ID NO: 313 - RNA, 23 nt, artificial sequence
  CCMV1_packaging signal
ggccauauguauucauggugguu

SEQ ID NO: 314 - RNA, 19 nt, artificial sequence
  CCMV1_packaging signal
gacauggguuacacagguc

SEQ ID NO: 315 - RNA, 13 nt, artificial sequence
  CCMV1_packaging signal
ugcguauuuugcg

SEQ ID NO: 316 - RNA, 22 nt, artificial sequence
  CCMV1_packaging signal
ggugcguauuuugcgggguacu

SEQ ID NO: 317 - RNA, 16 nt, artificial sequence
  CCMV1_packaging signal
ggguacuauuauguuc

SEQ ID NO: 318 - RNA, 19 nt, artificial sequence
  CCMV1_packaging signal
cgggguacuauuauguucg

SEQ ID NO: 319 - RNA, 24 nt, artificial sequence
  CCMV1_packaging signal
gguacuauuauguucgacggugcu

SEQ ID NO: 320 - RNA, 15 nt, artificial sequence
  CCMV1_packaging signal
uguuguuugacaacg

SEQ ID NO: 321 - RNA, 25 nt, artificial sequence
  CCMV1_packaging signal
caacgaaggcguuuuaccuuuguug

SEQ ID NO: 322 - RNA, 25 nt, artificial sequence
  CCMV1_packaging signal
ggcguuuuaccuuuguugaagugcc

SEQ ID NO: 323 - RNA, 28 nt, artificial sequence
  CCMV1_packaging signal
ucugaggucauuaaauuugauuucauga

SEQ ID NO: 324 - RNA, 30 nt, artificial sequence
  CCMV1_packaging signal
augaaugagagcacacuuucuuauauucau

SEQ ID NO: 325 - RNA, 21 nt, artificial sequence
  CCMV1_packaging signal
cuugguucauuuuugacugag

SEQ ID NO: 326 - RNA, 19 nt, artificial sequence
  CCMV1_packaging signal
guguuugguuugagaauau

SEQ ID NO: 327 - RNA, 23 nt, artificial sequence
  CCMV1_packaging signal
aagagauugcuuuucgauguuuu

SEQ ID NO: 328 - RNA, 20 nt, artificial sequence
  CCMV1_packaging signal
cuuuucgauguuuuaaggag

SEQ ID NO: 329 - RNA, 25 nt, artificial sequence
  CCMV1_packaging signal
agcgauagcaucuauucuguccgcu

SEQ ID NO: 330 - RNA, 9 nt, artificial sequence
  CCMV1_packaging signal
agaguuucu

SEQ ID NO: 331 - RNA, 30 nt, artificial sequence
  CCMV1_packaging signal
uuucuggcugguaaauucccuuggcugagg

SEQ ID NO: 332 - RNA, 29 nt, artificial sequence
  CCMV1_packaging signal
cguacaaagacaguuuuguuuuucugucg

SEQ ID NO: 333 - RNA, 17 nt, artificial sequence
  CCMV1_packaging signal
cugaggaguuucuucag

SEQ ID NO: 334 - RNA, 22 nt, artificial sequence
  CCMV1_packaging signal
acugaggaguuucuucagcagu

SEQ ID NO: 335 - RNA, 15 nt, artificial sequence
  CCMV1_packaging signal
gcauugaauuagagc

SEQ ID NO: 336 - RNA, 26 nt, artificial sequence
  CCMV1_packaging signal
uugaauuagagcuugaaucugcgcaa

SEQ ID NO: 337 - RNA, 12 nt, artificial sequence
  CCMV1_packaging signal
gcuugaaucugc

SEQ ID NO: 338 - RNA, 33 nt, artificial sequence
  CCMV1_packaging signal
aucgaugaggaggaauuucaagaugccaucgau

SEQ ID NO: 339 - RNA, 18 nt, artificial sequence
  CCMV1_packaging signal
aucaaggaauucucugau

SEQ ID NO: 340 - RNA, 34 nt, artificial sequence
  CCMV1_packaging signal
ucaaggaauucucugauuauugucgucgccuuga

SEQ ID NO: 341 - RNA, 19 nt, artificial sequence
  CCMV1_packaging signal
cgucgccuugacuguaacg

SEQ ID NO: 342 - RNA, 27 nt, artificial sequence
  CCMV1_packaging signal
cgauccuugaaacuuaucauaggguug

SEQ ID NO: 343 - RNA, 31 nt, artificial sequence
  CCMV1_packaging signal
gggcuuagguccgaaguuugaugaugagcuu

SEQ ID NO: 344 - RNA, 25 nt, artificial sequence
  CCMV1_packaging signal
gggaggcucuauucccucauaaucc

SEQ ID NO: 345 - RNA, 21 nt, artificial sequence
  CCMV1_packaging signal
ugcaugguuuaccgcgaugua

SEQ ID NO: 346 - RNA, 13 nt, artificial sequence
  CCMV1_packaging signal
cgcuuauuggucg

SEQ ID NO: 347 - RNA, 22 nt, artificial sequence
  CCMV1_packaging signal
agugucagucuguucuugcauu

SEQ ID NO: 348 - RNA, 20 nt, artificial sequence
  CCMV1_packaging signal
gagcaaauuucuuuuaaauc

SEQ ID NO: 349 - RNA, 19 nt, artificial sequence
  CCMV1_packaging signal
gcgagaugcaacuuuccgc

SEQ ID NO: 350 - RNA, 12 nt, artificial sequence
  CCMV1_packaging signal
gcaacuuuccgc

SEQ ID NO: 351 - RNA, 23 nt, artificial sequence
  CCMV1_packaging signal
gugauuugcaguuugacagucgc

SEQ ID NO: 352 - RNA, 18 nt, artificial sequence
  CCMV1_packaging signal
gcaagauguuauuuccgc

SEQ ID NO: 353 - RNA, 24 nt, artificial sequence
  CCMV1_packaging signal
agcaucaccuuuacaggugacgcu

SEQ ID NO: 354 - RNA, 27 nt, artificial sequence
  CCMV1_packaging signal
gggaaaaauucuauuugacaaugacuc

SEQ ID NO: 355 - RNA, 24 nt, artificial sequence
  CCMV1_packaging signal
ccgcccuuguuuccagggcuaagg

SEQ ID NO: 356 - RNA, 17 nt, artificial sequence
  CCMV1_packaging signal
gcccuuguuuccagggc

SEQ ID NO: 357 - RNA, 23 nt, artificial sequence
  CCMV1_packaging signal
gggcuaaggauuucccagagcuu

SEQ ID NO: 358 - RNA, 11 nt, artificial sequence
  CCMV1_packaging signal
gcuguauuggu

SEQ ID NO: 359 - RNA, 20 nt, artificial sequence
  CCMV1_packaging signal
acugaagaauauugcuuggu

SEQ ID NO: 360 - RNA, 25 nt, artificial sequence
  CCMV1_packaging signal
acucgacauaagauuaccuuugagu

SEQ ID NO: 361 - RNA, 30 nt, artificial sequence
  CCMV1_packaging signal
acauaagauuaccuuugaguaucuuuaugu

SEQ ID NO: 362 - RNA, 18 nt, artificial sequence
  CCMV1_packaging signal
guaucuuuauguugguau

SEQ ID NO: 363 - RNA, 22 nt, artificial sequence
  CCMV1_packaging signal
aguaucuuuauguugguaugcu

SEQ ID NO: 364 - RNA, 27 nt, artificial sequence
  CCMV1_packaging signal
agugugauucacuuacgaaucaguucu

SEQ ID NO: 365 - RNA, 10 nt, artificial sequence
  CCMV1_packaging signal
gccuuugggu

SEQ ID NO: 366 - RNA, 19 nt, artificial sequence
  CCMV1_packaging signal
uuuggguuuuacuccuuga

SEQ ID NO: 367 - RNA, 11 nt, artificial sequence
  CCMV1_packaging signal
ggguuuuacuc

SEQ ID NO: 368 - RNA, 12 nt, artificial sequence
  CCMV1_packaging signal
ggguuuuacucc

SEQ ID NO: 369 - RNA, 20 nt, artificial sequence
  CCMV1_packaging signal
uuuggguuuuacuccuugaa

SEQ ID NO: 370 - RNA, 29 nt, artificial sequence
  CCMV1_packaging signal
gaacccuucagaagaauucuucggaguuc

SEQ ID NO: 371 - RNA, 29 nt, artificial sequence
  CCMV2 packaging signal
gagagcgagguucaaucccuugucgacuc

SEQ ID NO: 372 - RNA, 10 nt, artificial sequence
  CCMV2 packaging signal
gauauuuuuc

SEQ ID NO: 373 - RNA, 35 nt, artificial sequence
  CCMV2 packaging signal
auuuuucuucuuuacuuccauuaauaugucuaagu

SEQ ID NO: 374 - RNA, 33 nt, artificial sequence
  CCMV2 packaging signal
cuuccauuaauaugucuaaguucauuccagaag

SEQ ID NO: 375 - RNA, 16 nt, artificial sequence
  CCMV2 packaging signal
ggcgauauucguaacc

SEQ ID NO: 376 - RNA, 22 nt, artificial sequence
  CCMV2 packaging signal
ccgaaucgauuaaugaaagugg

SEQ ID NO: 377 - RNA, 31 nt, artificial sequence
  CCMV2 packaging signal
uuaaugaaaguggaguugauacuucuguuga

SEQ ID NO: 378 - RNA, 10 nt, artificial sequence
  CCMV2 packaging signal
uucuguugaa

SEQ ID NO: 379 - RNA, 17 nt, artificial sequence
  CCMV2 packaging signal
gcuagcaaguuauaugc

SEQ ID NO: 380 - RNA, 18 nt, artificial sequence
  CCMV2 packaging signal
uagcaaguuauaugcaug

SEQ ID NO: 381 - RNA, 24 nt, artificial sequence
  CCMV2 packaging signal
aucccccuuuugaucaagcuagau

SEQ ID NO: 382 - RNA, 11 nt, artificial sequence
  CCMV2 packaging signal
ugguuucaccg

SEQ ID NO: 383 - RNA, 14 nt, artificial sequence
  CCMV2 packaging signal
gcaauguuugaugu

SEQ ID NO: 384 - RNA, 20 nt, artificial sequence
  CCMV2 packaging signal
cagagaggaguucgcgucug

SEQ ID NO: 385 - RNA, 24 nt, artificial sequence
  CCMV2 packaging signal
aguucgcgucuguugacucggauu

SEQ ID NO: 386 - RNA, 16 nt, artificial sequence
  CCMV2 packaging signal
gucuguugacucggau

SEQ ID NO: 387 - RNA, 30 nt, artificial sequence
  CCMV2 packaging signal
ccuggugagcccuguggaguucaggguggg

SEQ ID NO: 388 - RNA, 11 nt, artificial sequence
  CCMV2 packaging signal
ccgucauucgg

SEQ ID NO: 389 - RNA, 16 nt, artificial sequence
  CCMV2 packaging signal
caguuuaaaaucgcug

SEQ ID NO: 390 - RNA, 18 nt, artificial sequence
  CCMV2 packaging signal
ugauguugauugguaucg

SEQ ID NO: 391 - RNA, 17 nt, artificial sequence
  CCMV2 packaging signal
ccugaguuaaguauagg

SEQ ID NO: 392 - RNA, 9 nt, artificial sequence
  CCMV2 packaging signal
ggucauucc

SEQ ID NO: 393 - RNA, 8 nt, artificial sequence
  CCMV2 packaging signal
ucuguuga

SEQ ID NO: 394 - RNA, 25 nt, artificial sequence
  CCMV2 packaging signal
cuuaucuuaaucauuccgguauagg

SEQ ID NO: 395 - RNA, 13 nt, artificial sequence
  CCMV2 packaging signal
ggacuugaguacc

SEQ ID NO: 396 - RNA, 12 nt, artificial sequence
  CCMV2 packaging signal
gacaguuuuguc

SEQ ID NO: 397 - RNA, 12 nt, artificial sequence
  CCMV2 packaging signal
caguuuugucug

SEQ ID NO: 398 - RNA, 13 nt, artificial sequence
  CCMV2 packaging signal
ccaguugucucgg

SEQ ID NO: 399 - RNA, 10 nt, artificial sequence
  CCMV2 packaging signal
agcuguugcu

SEQ ID NO: 400 - RNA, 12 nt, artificial sequence
  CCMV2 packaging signal
cggcuuguuucg

SEQ ID NO: 401 - RNA, 23 nt, artificial sequence
  CCMV2 packaging signal
uucaucuugaguuccaaagagag

SEQ ID NO: 402 - RNA, 16 nt, artificial sequence
  CCMV2 packaging signal
gagagauauuguuguc

SEQ ID NO: 403 - RNA, 12 nt, artificial sequence
  CCMV2 packaging signal
gauauuguuguc

SEQ ID NO: 404 - RNA, 27 nt, artificial sequence
  CCMV2 packaging signal
gucauuggguuuuccagccccuuugac

SEQ ID NO: 405 - RNA, 27 nt, artificial sequence
  CCMV2 packaging signal
gucauuggguuuuccagccccuuugac

SEQ ID NO: 406 - RNA, 13 nt, artificial sequence
  CCMV2 packaging signal
ugugauuuccaua

SEQ ID NO: 407 - RNA, 16 nt, artificial sequence
  CCMV2 packaging signal
gcuggaguuaacaugc

SEQ ID NO: 408 - RNA, 10 nt, artificial sequence
  CCMV2 packaging signal
cuuauuuugg

SEQ ID NO: 409 - RNA, 11 nt, artificial sequence
  CCMV2 packaging signal
cuuauuuuggg

SEQ ID NO: 410 - RNA, 10 nt, artificial sequence
  CCMV2 packaging signal
uacuuuggug

SEQ ID NO: 411 - RNA, 17 nt, artificial sequence
  CCMV2 packaging signal
cuguuaauuuguaaaag

SEQ ID NO: 412 - RNA, 9 nt, artificial sequence
  CCMV2 packaging signal
uguuuuuca

SEQ ID NO: 413 - RNA, 23 nt, artificial sequence
  CCMV2 packaging signal
aaucucuguuuaauauggaaauu

SEQ ID NO: 414 - RNA, 11 nt, artificial sequence
  CCMV2 packaging signal
acguuuguagu

SEQ ID NO: 415 - RNA, 32 nt, artificial sequence
  CCMV2 packaging signal
uuguaguaaguuucuuuuagaaacugaaauga

SEQ ID NO: 416 - RNA, 18 nt, artificial sequence
  CCMV2 packaging signal
ugaguuguuaagagccca

SEQ ID NO: 417 - RNA, 18 nt, artificial sequence
  CCMV2 packaging signal
guccuuuugugauaggau

SEQ ID NO: 418 - RNA, 29 nt, artificial sequence
  CCMV2 packaging signal
uuaugcaaguuuguggcucucaaguauaa

SEQ ID NO: 419 - RNA, 25 nt, artificial sequence
  CCMV2 packaging signal
ucagaguagccauugcugcuuucgg

SEQ ID NO: 420 - RNA, 9 nt, artificial sequence
  CCMV2 packaging signal
gcuuucggc

SEQ ID NO: 421 - RNA, 32 nt, artificial sequence
  CCMV2 packaging signal
gcuacuacucagaaaauuucuugagauuuugc

SEQ ID NO: 422 - RNA, 24 nt, artificial sequence
  CCMV2 packaging signal
agauuuugcgaauguuaugcgacu

SEQ ID NO: 423 - RNA, 10 nt, artificial sequence
  CCMV2 packaging signal
uucuuuggaa

SEQ ID NO: 424 - RNA, 12 nt, artificial sequence
  CCMV2 packaging signal
uucuuccuugaa

SEQ ID NO: 425 - RNA, 33 nt, artificial sequence
  CCMV2 packaging signal
uaaauaauguuggucacauuuaagacuuguuua

SEQ ID NO: 426 - RNA, 13 nt, artificial sequence
  CCMV2 packaging signal
gacuuguuuaguc

SEQ ID NO: 427 - RNA, 31 nt, artificial sequence
  CCMV2 packaging signal
uguuuaguccacauuaggacugguucuaaca

SEQ ID NO: 428 - RNA, 14 nt, artificial sequence
  CCMV2 packaging signal
guugguuugcuuac

SEQ ID NO: 429 - RNA, 29 nt, artificial sequence
  CCMV2 packaging signal
ucaagcugccuuugaguuuuacuccuuga

SEQ ID NO: 430 - RNA, 31 nt, artificial sequence
  CCMV3 packaging signal
caacuuucaaacuuuauaguuuauguaguug

SEQ ID NO: 431 - RNA, 23 nt, artificial sequence
  CCMV3 packaging signal
gacacaucgguuuuugaagcauc

SEQ ID NO: 432 - RNA, 31 nt, artificial sequence; identical to SEQ ID NO: 430
  CCMV3 packaging signal

SEQ ID NO: 433 - RNA, 23 nt, artificial sequence
  CCMV3 packaging signal
gacacaucgguuuuugaagcauc

SEQ ID NO: 434 - RNA, 19 nt, artificial sequence
  CCMV3 packaging signal
aguaggcuguuaccugacu

SEQ ID NO: 435 - RNA, 9 nt, artificial sequence
  CCMV3 packaging signal
guucuuugc

SEQ ID NO: 436 - RNA, 26 nt, artificial sequence
  CCMV3 packaging signal
gaugucuaacacuacuuuuagaccuu

SEQ ID NO: 437 - RNA, 15 nt, artificial sequence
  CCMV3 packaging signal
gaccuuuuacugguu

SEQ ID NO: 438 - RNA, 34 nt, artificial sequence
  CCMV3 packaging signal
ggaugauaugucguuguuacagucacuuuuuucc

SEQ ID NO: 439 - RNA, 8 nt, artificial sequence
  CCMV3 packaging signal
guuguuac

SEQ ID NO: 440 - RNA, 15 nt, artificial sequence
  CCMV3 packaging signal
gcugguuuuucuugu

SEQ ID NO: 441 - RNA, 14 nt, artificial sequence
  CCMV3 packaging signal
uuuucuugugagga

SEQ ID NO: 442 - RNA, 22 nt, artificial sequence
  CCMV3 packaging signal
uagacacagauguuucgguuug

SEQ ID NO: 443 - RNA, 16 nt, artificial sequence
  CCMV3 packaging signal
caugcguauuggucug

SEQ ID NO: 444 - RNA, 27 nt, artificial sequence
  CCMV3 packaging signal
gagucaugcguauuggucugcgaacuu

SEQ ID NO: 445 - RNA, 35 nt, artificial sequence
  CCMV3 packaging signal
uaugagucaugcguauuggucugcgaacuuucgua

SEQ ID NO: 446 - RNA, 35 nt, artificial sequence
  CCMV3 packaging signal
uggucugcgaacuuucguaguaaaccuaauaacua

SEQ ID NO: 447 - RNA, 29 nt, artificial sequence
  CCMV3 packaging signal
auguggaacccuuugacagguugaaacgu

SEQ ID NO: 448 - RNA, 11 nt, artificial sequence
  CCMV3 packaging signal
ccuuugacagg

SEQ ID NO: 449 - RNA, 17 nt, artificial sequence
  CCMV3 packaging signal
ucaugguuaucuauugg

SEQ ID NO: 450 - RNA, 22 nt, artificial sequence
  CCMV3 packaging signal
ugguuaucuauuggguaaacca

SEQ ID NO: 451 - RNA, 21 nt, artificial sequence
  CCMV3 packaging signal
ccguugcggggcuuccgacgg

SEQ ID NO: 452 - RNA, 8 nt, artificial sequence
  CCMV3 packaging signal
gggcuucc

SEQ ID NO: 453 - RNA, 8 nt, artificial sequence
  CCMV3 packaging signal
uauguuua

SEQ ID NO: 454 - RNA, 26 nt, artificial sequence
  CCMV3 packaging signal
uugauaguaauuuaucaugucuacag

SEQ ID NO: 455 - RNA, 18 nt, artificial sequence
  CCMV3 packaging signal
acagggaaguuaacucgu

SEQ ID NO: 456 - RNA, 12 nt, artificial sequence
  CCMV3 packaging signal
cuguuauuguag

SEQ ID NO: 457 - RNA, 13 nt, artificial sequence
  CCMV3 packaging signal
guuauuguagaac

SEQ ID NO: 458 - RNA, 24 nt, artificial sequence
  CCMV3 packaging signal
guggaccgccucuugugcggcugc

SEQ ID NO: 459 - RNA, 14 nt, artificial sequence
  CCMV3 packaging signal
gccucuugugcggc

SEQ ID NO: 460 - RNA, 19 nt, artificial sequence
  CCMV3 packaging signal
uagguagaguuuuauuaug

SEQ ID NO: 461 - RNA, 31 nt, artificial sequence
  CCMV3 packaging signal
agguagaguuuuauuauggcuuggguugcuu

SEQ ID NO: 462 - RNA, 10 nt, artificial sequence
  CCMV3 packaging signal
ggcuuggguu

SEQ ID NO: 463 - RNA, 13 nt, artificial sequence
  CCMV3 packaging signal
ggcuuggguugcu

SEQ ID NO: 464 - RNA, 11 nt, artificial sequence
  CCMV3 packaging signal
gguugcuuccc

SEQ ID NO: 465 - RNA, 25 nt, artificial sequence
  CCMV3 packaging signal
uggcuuggguugcuucccaguguua

SEQ ID NO: 466 - RNA, 22 nt, artificial sequence
  CCMV3 packaging signal
uuuggagguugagcaugucaga

SEQ ID NO: 467 - RNA, 9 nt, artificial sequence
  CCMV3 packaging signal
gacucuuuc

SEQ ID NO: 468 - RNA, 18 nt, artificial sequence
  CCMV3 packaging signal
uggccuacuugaaggcua

SEQ ID NO: 469 - RNA, 10 nt, artificial sequence
  CCMV3 packaging signal
ucguuguuga

SEQ ID NO: 470 - RNA, 25 nt, artificial sequence
  CCMV3 packaging signal
gguaaucguuguugaaacgucuucc

SEQ ID NO: 471 - RNA, 11 nt, artificial sequence
  CCMV3 packaging signal
gguuuuacucc

SEQ ID NO: 472 - RNA, 31 nt, artificial sequence
  TCV packaging signal
ggagcuggucgggagggagacucaaaucucc

SEQ ID NO: 473 - RNA, 40 nt, artificial sequence
  TCV packaging signal
ucuacagguuauccaagaacgggaugaggggcugcaaaga

SEQ ID NO: 474 - RNA, 43 nt, artificial sequence
  TCV packaging signal
cuguuuuaaacaagaaaagaaaugaagguucugcuagucacgg

SEQ ID NO: 475 - RNA, 37 nt, artificial sequence; identical to SEQ ID NO: 66
  TCV packaging signal

SEQ ID NO: 476 - RNA, 22 nt, artificial sequence
  PS STNV1
aguaaagacaggaaacuuuacu

SEQ ID NO: 477 - RNA, 17 nt, artificial sequence
  PS STNV1
acaacagaacaacaggc

SEQ ID NO: 478 - RNA, 12 nt, artificial sequence
  PS STNV1
cgcaacaaugcg

SEQ ID NO: 479 - RNA, 13 nt, artificial sequence
  PS STNV1
ugauaaauacaca

SEQ ID NO: 480 - RNA, 15 nt, artificial sequence
  PS STNV1
gcauaaaagguuugc

SEQ ID NO: 481 - RNA, 15 nt, artificial sequence
  PS STNV1
cagggaacaccaaug

SEQ ID NO: 482 - RNA, 16 nt, artificial sequence
  PS STNV1
acaguacaaaaucugu

SEQ ID NO: 483 - RNA, 18 nt, artificial sequence
  PS STNV1
auaauccaaggagaugau

SEQ ID NO: 484 - RNA, 17 nt, artificial sequence
  PS STNV1
caaccagagaaguggug

SEQ ID NO: 485 - RNA, 16 nt, artificial sequence
  PS STNV1
cacguacgaggcacug

SEQ ID NO: 486 - RNA, 16 nt, artificial sequence
  PS STNV1
uucgugauaacaugaa

SEQ ID NO: 487 - RNA, 16 nt, artificial sequence
  PS STNV1
gugggaccacucccac

SEQ ID NO: 488 - RNA, 14 nt, artificial sequence
  PS STNV1
uguugaacacugcg

SEQ ID NO: 489 - RNA, 20 nt, artificial sequence
  PS STNV1
uauaacccaaucacguugca

SEQ ID NO: 490 - RNA, 14 nt, artificial sequence
  PS STNV1
uacucaaggaugua

SEQ ID NO: 491 - RNA, 18 nt, artificial sequence
  PS STNV1
agaucggauaauuaaccu

SEQ ID NO: 492 - RNA, 13 nt, artificial sequence
  PS STNV1
ccaggacaacugg

SEQ ID NO: 493 - RNA, 16 nt, artificial sequence
  PS STNV1
ggcuguagcagccucc

SEQ ID NO: 494 - RNA, 16 nt, artificial sequence
  PS STNV1
gcgcugaaagaugcgu

SEQ ID NO: 495 - RNA, 14 nt, artificial sequence
  PS STNV1
uaagcagaaaucca

SEQ ID NO: 496 - RNA, 20 nt, artificial sequence
  PS STNV1
gguggaaagcagucccagcu

SEQ ID NO: 497 - RNA, 19 nt, artificial sequence
  PS STNV1
uagucuaaaugagacguug

SEQ ID NO: 498 - RNA, 17 nt, artificial sequence
  PS STNV1
ugccauuaguaggucua

SEQ ID NO: 499 - RNA, 19 nt, artificial sequence
  PS STNV1
ugcaacaagaauaugugcg

SEQ ID NO: 500 - RNA, 18 nt, artificial sequence
  PS STNV1
gcgguauauuaagugcgc

SEQ ID NO: 501 - RNA, 14 nt, artificial sequence
  PS STNV1
guuuggaccagggc

SEQ ID NO: 502 - RNA, 15 nt, artificial sequence
  PS STNV1
gcuuuaggagaugau

SEQ ID NO: 503 - RNA, 21 nt, artificial sequence
  PS STNV1
guauaguuauuagacaaaugc

SEQ ID NO: 504 - RNA, 21 nt, artificial sequence
  PS STNV1
ggccaagcgaagaaccucauc

SEQ ID NO: 505 - RNA, 22 nt, artificial sequence
  PS STNV1
aaauuugguaccauccaaacuu

SEQ ID NO: 506 - RNA, 15 nt, artificial sequence
  PS STNV2
aagacaggaaacuuu

SEQ ID NO: 507 - RNA, 13 nt, artificial sequence
  PS STNV2
ugacaaaacguca

SEQ ID NO: 508 - RNA, 15 nt, artificial sequence
  PS STNV2
aaccgcaagagcguu

SEQ ID NO: 509 - RNA, 15 nt, artificial sequence
  PS STNV2
ugcguaguauuguug

SEQ ID NO: 510 - RNA, 14 nt, artificial sequence
  PS STNV2
gagcagaagcgauu

SEQ ID NO: 511 - RNA, 14 nt, artificial sequence
  PS STNV2
uacgaacaccaaca

SEQ ID NO: 512 - RNA, 23 nt, artificial sequence
  PS STNV2
gucacuacagcagguaccgugau

SEQ ID NO: 513 - RNA, 14 nt, artificial sequence
  PS STNV2
accugagcaacaac

SEQ ID NO: 514 - RNA, 18 nt, artificial sequence
  PS STNV2
gcaaggagaugaccuugu

SEQ ID NO: 515 - RNA, 17 nt, artificial sequence
  PS STNV2
gauuaagaccauacacc

SEQ ID NO: 516 - RNA, 17 nt, artificial sequence
  PS STNV2
gguguacaggaauuacc

SEQ ID NO: 517 - RNA, 16 nt, artificial sequence
  PS STNV2
uucgugacaacaccaa

SEQ ID NO: 518 - RNA, 16 nt, artificial sequence
  PS STNV1
ggggacuacaccggcu

SEQ ID NO: 519 - RNA, 23 nt, artificial sequence
  PS STNV2
gugcuaguauaacaucccaguau

SEQ ID NO: 520 - RNA, 19 nt, artificial sequence
  PS STNV2
cagcaaaagagguucacug

SEQ ID NO: 521 - RNA, 21 nt, artificial sequence
  PS STNV1
ugccguugauaagaaacggcg

SEQ ID NO: 522 - RNA, 23 nt, artificial sequence
  PS STNV2
gcgauauuuuacaacggugcugc

SEQ ID NO: 523 - RNA, 14 nt, artificial sequence
  PS STNV2
cauuggaucacaug

SEQ ID NO: 524 - RNA, 21 nt, artificial sequence
  PS STNV2
cuggacaguaugaugugacag

SEQ ID NO: 525 - RNA, 18 nt, artificial sequence
  PS STNV2
ucaugaugaugauaguga

SEQ ID NO: 526 - RNA, 16 nt, artificial sequence
  PS STNV2
acgcugaaagaugcgu

SEQ ID NO: 527 - RNA, 12 nt, artificial sequence
  PS STNV2
ggacaguagucc

SEQ ID NO: 528 - RNA, 12 nt, artificial sequence
  PS STNV2
aacuaguaaauc

SEQ ID NO: 529 - RNA, 19 nt, artificial sequence
  PS STNV1
gaccgggagaaaaccagcu

SEQ ID NO: 530 - RNA, 17 nt, artificial sequence
  PS STNV2
guggaacgaggccccgc

SEQ ID NO: 531 - RNA, 12 nt, artificial sequence
  PS STNV2
guggaaaaccau

SEQ ID NO: 532 - RNA, 15 nt, artificial sequence
  PS STNV2
gugcaacaaugcugu

SEQ ID NO: 533 - RNA, 16 nt, artificial sequence
  PS STNV2
cucaacaucacuucaa

SEQ ID NO: 534 - RNA, 13 nt, artificial sequence
  PS STNV2
augucacaagaau

SEQ ID NO: 535 - RNA, 21 nt, artificial sequence
  PS STNV2
guauagugacuagacaaaugc

SEQ ID NO: 536 - RNA, 13 nt, artificial sequence
  PS STNV2
gccucaacaaggu

SEQ ID NO: 537 - RNA, 15 nt, artificial sequence
  PS STNV2
ugcauaggagaugug

SEQ ID NO: 538 - RNA, 13 nt, artificial sequence
  PS STNVc
uuauacaaaguag

SEQ ID NO: 539 - RNA, 20 nt, artificial sequence
  PS STNVc
ucaugguauuaggguggugg

SEQ ID NO: 540 - RNA, 12 nt, artificial sequence
  PS STNVc
cugaaagauuaa

SEQ ID NO: 541 - RNA, 16 nt, artificial sequence
  PS STNVc
aacaugacuaaacguc

SEQ ID NO: 542 - RNA, 18 nt, artificial sequence
  PS STNVc
acaaacaacuagaucugu

SEQ ID NO: 543 - RNA, 16 nt, artificial sequence
  PS STNVc
uguuagaucacucacg

SEQ ID NO: 544 - RNA, 20 nt, artificial sequence
  PS STNVc
acgugcggaacaucauacgu

SEQ ID NO: 545 - RNA, 14 nt, artificial sequence
  PS STNVc
accaaacgauuugu

SEQ ID NO: 546 - RNA, 19 nt, artificial sequence
  PS STNVc
ucuuaacaguaccgcugga

SEQ ID NO: 547 - RNA, 17 nt, artificial sequence
  PS STNVc
caucauacaaggcgaug

SEQ ID NO: 548 - RNA, 17 nt, artificial sequence
  PS STNVc
uggagauaagauucgua

SEQ ID NO: 549 - RNA, 20 nt, artificial sequence
  PS STNVc
agcgacugccauaacaaauu

SEQ ID NO: 550 - RNA, 15 nt, artificial sequence
  PS STNVc
guuuaaggauaacac

SEQ ID NO: 551 - RNA, 17 nt, artificial sequence
  PS STNVc
ucgugguaccacuccaa

SEQ ID NO: 552 - RNA, 17 nt, artificial sequence
  PS STNVc
gacugaaguacuuaacu

SEQ ID NO: 553 - RNA, 14 nt, artificial sequence
  PS STNVc
ggcccaauacaacc

SEQ ID NO: 554 - RNA, 14 nt, artificial sequence
  PS STNVc
acuacagcauaggu

SEQ ID NO: 555 - RNA, 16 nt, artificial sequence
  PS STNVc
uccucaaggauguuga

SEQ ID NO: 556 - RNA, 14 nt, artificial sequence
  PS STNVc
cugucaggagagag

SEQ ID NO: 557 - RNA, 17 nt, artificial sequence
  PS STNVc
uuggugaugacgcaugg

SEQ ID NO: 558 - RNA, 16 nt, artificial sequence
  PS STNVc
guuucuauaauggaac

SEQ ID NO: 559 - RNA, 13 nt, artificial sequence
  PS STNVc
ggagcaauauucc

SEQ ID NO: 560 - RNA, 12 nt, artificial sequence
  PS STNVc
gguuacgaggcu

SEQ ID NO: 561 - RNA, 18 nt, artificial sequence
  PS STNVc
uuugaaaaaucauucaaa

SEQ ID NO: 562 - RNA, 14 nt, artificial sequence
  PS STNVc
augucaccagacgu

SEQ ID NO: 563 - RNA, 18 nt, artificial sequence
  PS STNVc
aucccugaaccaggcugu

SEQ ID NO: 564 - RNA, 16 nt, artificial sequence
  PS STNVc
cugcuaggacgaaugg

SEQ ID NO: 565 - RNA, 16 nt, artificial sequence
  PS STNVc
uaauacacaagguucg

SEQ ID NO: 566 - RNA, 15 nt, artificial sequence
  PS STNVc
auaguaggaagccgu

SEQ ID NO: 567 - RNA, 18 nt, artificial sequence
  PS STNVc
gguaauuuacgaaagacc

SEQ ID NO: 568 - RNA, 16 nt, artificial sequence
  PS STNVc
uucuggcauaauugag

SEQ ID NO: 569 - RNA, 17 nt, artificial sequence
  PS STNVc
gauaaaaggaguugauc

SEQ ID NO: 570 - RNA, 15 nt, artificial sequence
  PS STNVc
uguggaagaauucug

SEQ ID NO: 571 - RNA, 18 nt, artificial sequence
  PS STNVc
ggggaguacuacaccuuc

SEQ ID NO: 572 - RNA, 14 nt, artificial sequence
  PS STNVc
cacuaaggacuaug

SEQ ID NO: 573 - RNA, 30 nt, artificial sequence
  PS HIV
gcugacacaggacacagcaaucaggucagc

SEQ ID NO: 574 - RNA, 26 nt, artificial sequence
  PS HIV
ccccucgucacaauaaagauaggggg

SEQ ID NO: 575 - RNA, 16 nt, artificial sequence
  PS HIV
uaguguuacgaaacug

SEQ ID NO: 576 - RNA, 14 nt, artificial sequence
  PS HIV
gccuguccaaaggu

SEQ ID NO: 577 - RNA, 16 nt, artificial sequence
  PS HIV
uaagaccaaugacuua

SEQ ID NO: 578 - RNA, 21 nt, Artificial Sequence
  PS HPeV
uaaaaugucuggugagaugug

SEQ ID NO: 579 - RNA, 39 nt, Artificial Sequence
  PS HPeV
ucccugguuuccuuuuauuguuaauauugacauuaugga

SEQ ID NO: 580 - RNA, 27 nt, Artificial Sequence
  PS HPeV
gguguuguaaguucuguugauucuacc

SEQ ID NO: 581 - RNA, 26 nt, Artificial Sequence
  PS HPeV
auuggagguaauuuguuaacuaaagu

SEQ ID NO: 582 - RNA, 22 nt, Artificial Sequence
  PS HPeV
auuaccuaaaguuuuuugggau

SEQ ID NO: 583 - RNA, 19 nt, Artificial Sequence
  PS HPeV
uuccacauguuuugaugaa

SEQ ID NO: 584 - RNA, 22 nt, Artificial Sequence
  PS HPeV
ugaauguuuuuguuaacaguua

SEQ ID NO: 585 - RNA, 20 nt, Artificial Sequence
  PS HPeV
gguucauuaguuuuaagauu

SEQ ID NO: 586 - RNA, 23 nt, Artificial Sequence
  PS HPeV
cugguucuguuguuacauuccag

SEQ ID NO: 587 - RNA, 22 nt, Artificial Sequence
  PS HPeV
uucucaauuuuaggucgaugaa

SEQ ID NO: 588 - RNA, 32 nt, Artificial Sequence
  PS HPeV
uuaucacuguuguuugcuuauuuuacugguga

SEQ ID NO: 589 - RNA, 28 nt, Artificial Sequence
  PS HPeV
agucuugguuauuugaugugcaagcccu

SEQ ID NO: 590 - RNA, 22 nt, Artificial Sequence
  PS HPeV
uugagguuuaucuuagccugag

SEQ ID NO: 591 - RNA, 24 nt, Artificial Sequence
  PS HPeV
uggauaaugauuuagucaaguuca

SEQ ID NO: 592 - RNA, 17 nt, Artificial Sequence
  PS HPeV
gacauuauuguugaguc

SEQ ID NO: 593 - RNA, 19 nt, Artificial Sequence
  PS HPeV
uuaaugguguuuuuacuaa

SEQ ID NO: 594 - RNA, 25 nt, Artificial Sequence
  PS HPeV
uccaugcucaguuuuguugagagga

SEQ ID NO: 595 - RNA, 25 nt, Artificial Sequence
  PS HPeV
uuaguauacuuuuguugguaacaaa

SEQ ID NO: 596 - RNA, 29 nt, Artificial Sequence
  PS HPeV
agcugguuauaguuuuguuaaaucuggcu

SEQ ID NO: 597 - RNA, 30 nt, Artificial Sequence
  PS HPeV
uugugaaguugauuauugcauuguuuacag

SEQ ID NO: 598 - RNA, 20 nt, Artificial Sequence
  PS HPeV
ugauguguauuuacacuaca

SEQ ID NO: 599 - RNA, 23 nt, Artificial Sequence
  PS HPeV
aagauuaauguuuuguuuuucuu

SEQ ID NO: 600 - RNA, 23 nt, Artificial Sequence
  PS HPeV
cuggaaguguaguaacauuccag

SEQ ID NO: 601 - RNA, 23 nt, Artificial Sequence
  PS HPeV
aagacgaaugaaacguucgucuu

(19) United States
(12) Patent Application Publication
(10) Pub. No.: US 2016/0326529 A1
(43) Pub. Date: Nov. 10, 2016

(54) ANTI-VIRAL THERAPY

(71) Applicants: THE UNIVERSITY OF YORK, York (GB); UNIVERSITY OF LEEDS, Leeds (GB); UNIVERSITY OF HELSINKI, Helsinki (FI)

(72) Inventors: Reidun TWAROCK, York (GB); Eric DYKEMAN, York (GB); Peter STOCKLEY, Leeds (GB); Simon WHITE, Leeds (GB); Amy BARKER, Leeds (GB); Nikesh PATEL, Leeds (GB); Sarah BUTCHER, Helsinki (FI); Shabih SHAKEEL, Helsinki (FI); Mark HARRIS, Leeds (GB); Hazel STEWART, Leeds (GB)

(73) Assignees: The University of York, York (GB); University of Leeds, Leeds (GB); University of Helsinki, Helsinki (FI)

(21) Appl. No.: 14/916,945

(22) PCT Filed: Sep. 5, 2014
(86) PCT No.: PCT/GB2014/052696

§ 371 Date: Mar. 4, 2016

(30) Foreign Application Priority Data
Sep. 5, 2013 (GB) 1315785.4

(51) Int. Cl. C12N 15/115; C12N 15/82; G01N 33/53; C12N 15/10; A61K 31/7105; A01N 57/16

(52) U.S. Cl. CPC C12N 15/115; A61K 31/7105; A01N 57/16; G01N 33/5308; C12N 15/1048; C12N 15/8283; C12N 2310/531; C12N 2320/31; C12N 2310/13; C12Q 2600/136; C12N 2310/16; C12N 2330/31

(57) ABSTRACT

The disclosure relates to anti-viral agents that mimic or inhibit packaging singles of RNA viruses that function in viral capsid formation and their use in the control of viral infection.

### CROSS REFERENCE TO RELATED APPLICATIONS

This is the U.S. National Stage of International Application No. PCT/GB2014/052696, filed Sep. 5, 2014, which was published in English under PCT Article 21(2), which in turn claims the benefit of Great Britain Application No. 1315785.4, filed Sep. 5, 2013.

### FIELD OF THE INVENTION

The disclosure relates to anti-viral agents that either mimic or bind to packaging signals of RNA viruses that function in viral capsid formation; pharmaceutical and plant viral control compositions for use in the treatment of viral infections; methods to treat viral infections; and methods to screen for packaging signals in viral RNA genomes.

### BACKGROUND OF THE INVENTION

Several diseases in humans, animals and plants are caused by so called RNA viruses. Single-stranded RNA viruses are divided into three groups: Positive-sense ssRNA viruses (Group IV), negative-sense ssRNA viruses (Group V) and retroviruses (Group VI). On infection, the viral RNA enters the host cells and, dependent on the type of virus, RNA is directly translated (Group IV) into the viral proteins necessary for replication or is, prior to translation, transcribed into a more suitable form of RNA by an RNA-dependent RNA polymerase (Group V). Group VI RNA viruses utilise a virally encoded reverse transcriptase to produce DNA from the RNA genome, which is often integrated into the host genome and so replicated and transcribed by the host. Group IV viruses include the picornaviruses, such as polio, foot & mouth disease virus, human rhinovirus, Coxsackievirus B, and other enteroviruses, as well as the alpha viruses, including Chikungunya and West Nile virus and the hepatitis viruses A, C-E. Hepatitis B is a dsDNA virus but co-assembles via a pro-genomic ssRNA.

RNA viruses have a simple structure comprising RNA enclosed in a protein shell called a capsid, (i.e. they form a nucleocapsid). The formation of a protein container that encapsulates and provides protection for the viral genome is a vital step in most viral life-cycles (M. G. Rossmann and J. E. Johnson, Icosahedral RNA virus structure Annu Rev Biochem. 58, 533-73 (1989)). It is a prime example of molecular self-assembly, exemplifying the fundamental principles underlying the formation of protein nano-containers that are important both in virology (Isolation of an asymmetric RNA uncoating intermediate for a single-stranded RNA plant virus Bakker S E, Ford R J, Barker A M, Robottom J, Saunders K, Pearson A R, Ranson N A, Stockley P G. J Mol Biol. 2012 Mar. 16; 417(1-2):65-78.), and for applications in bionanotechnology (M. Wu, W. L. Brown, and P. G. Stockley, Cell-specific delivery of bacteriophage-encapsidated ricin A chain. Bioconjug Chem. 6, 587-95 (1995)) and synthetic biology (N. F. Steinmetz, V. Hong, E. D. Spoerke, P. Lu, K. Breitenkamp, M. G. Finn, and M. Manchester, Buckyballs meet viral nanoparticles: candidates for biomedicine J Am Chem Soc. 131, 17093-5 (2009)).

Methods and compositions for controlling capsid formation are disclosed in US2013156818. Similarly, US2013/0165489 discloses small molecule modulators of HIV-1 capsid stability.

While the mechanisms of (nucleo-) capsid formation and genome encapsulation vary across viral families, there are a number of common features that can be characterised collectively. For example, pro-capsid formation may occur via the self- or assisted assembly of protein subunits and be followed by the introduction of the genomic material via a packaging motor, as seen in many double-stranded DNA viruses (S. Sun, S. Gao, K. Kondabagil, Y. Xiang, M. G. Rossmann, and V. B. Rao. Structure and function of the small terminase component of the DNA packaging machine in T4-like bacteriophages. Proc Natl Acad Sci USA. 109, 817-22 (2012)). Alternatively, capsid assembly may follow a co-assembly process involving protein subunits and the viral genome, a phenomenon occurring in many single-stranded RNA viruses [5,6]. These latter comprise one of the largest viral families and include major human, animal and plant pathogens.

In contrast to bacterial infections, once a subject has contracted a virus there is little that can be done to cure the patient. Viruses cause debilitating diseases in humans which can ultimately result in the death of the infected subject. The detrimental effect of viruses is not just restricted to human related illnesses, viruses cause also many important animal and plant diseases, causing huge losses of animal related products such as meat or diary, or resulting in severely reduced crop yields.

Vaccination is the most effective form of disease prevention and has been successfully developed for some viral diseases such as influenza, hepatitis B, polio or measles. Vaccination is the administration of antigenic material to stimulate an individual's immune system to develop adaptive immunity to a pathogen. The active agent of a vaccine may be, for example, an inactivated form of the pathogen, or highly immunogenic components of the pathogen. Although vaccines provide effective protection against many diseases, and have almost eradicated diseases such as polio, measles and tetanus from many parts of the world, some viral infections such as HIV are less susceptible to vaccines and moreover, RNA viruses have enormously high mutation rates, making the development of vaccines difficult and reducing their effectiveness.

Additionally, there are no vaccines available for the use in plants, and control of plant viruses requires typically a great amount of effort such as the development of disease resistant plants or employing carefully controlled growth conditions to minimise infections.

We disclose that single-stranded RNA viruses assemble their capsids with great fidelity and efficiency at low concentrations using a mechanism that involves multiple coat protein (CP)-genomic RNA interactions at sites consisting of sequence-degenerate short fragments of RNA called Packaging Signals (PSs) [1-2].

This disclosure relates to an anti-viral therapy comprising: 1) the use of small organic compounds or example nucleic acid based compounds, ablating PS-CP interaction and therefore preventing or severely reducing capsid assembly; or 2) the production of decoy RNAs in plants displaying PSs on non-genomic and therefore non-pathogenic RNAs. Defective capsid assembly has several beneficial effects such as lower viral titres and therefore reducing symptoms caused by a viral infection, exposing conserved protein epitopes in animal viruses thus acting as good adjuvants for immune recognition and exposing viral genomes to RNA silencing in plants. Since PSs function collectively during assembly and are also part of the coding of viral genes, development of resistances are reduced when compared to methods that target the functions of individual viral proteins.

### STATEMENTS OF THE INVENTION

According to an aspect of the invention there is provided an anti-viral agent effective in controlling the formation of the viral capsid of an RNA virus wherein said agent is a nucleic acid stem-loop structure and comprises:
i) a nucleic acid loop domain comprising one or more nucleotide bases comprising a nucleotide binding motif for one or more capsid assembly domains in a viral capsid protein; and
ii) a nucleic acid stem domain wherein the stem domain is at least two nucleotide bases in length which over all or part of its length forms a double-stranded region by intramolecular complementary base pairing,
   wherein said anti-viral agent inhibits the formation of the viral capsid.

In a preferred embodiment of the invention said loop domain comprises at least 4 nucleotides; preferably said loop domain comprises between 4 and 8 nucleotides.

In a preferred embodiment of the invention said stem domain comprises at least 2 nucleotides wherein at least one nucleotide is base paired with a complementary base.

In a preferred embodiment of the invention said stem domain comprises between 2 and 13 nucleotides which are base paired by intramolecular complementary base paring.

In a preferred embodiment of the invention said loop domain comprises at least one uracil base; preferably at least 2, 3 or 4 uracil bases.

In a preferred embodiment of the invention said RNA virus is an animal virus.

In a preferred embodiment of the invention said animal RNA virus is a human virus.

In a preferred embodiment of the invention said human virus is a hepatitis virus; preferably hepatitis B virus [HBV] or hepatitis C virus [HCV].

In a preferred embodiment of the invention said human virus is hepatitis B virus [HBV].

In a preferred embodiment of the invention said nucleic acid based anti-viral agent comprises:
i) a nucleic acid loop domain comprising 5 to 12 nucleotide bases comprising an A-G nucleotide base rich binding motif for one or more HBV capsid assembly domains in a HBV capsid protein; and
ii) a nucleic acid stem domain wherein the stem domain comprises 4 to 30 nucleotides in length which over all or part of its length forms a double-stranded region by intramolecular complementary base pairing, wherein said anti-viral agent inhibits the formation of the HBV capsid.

In a preferred embodiment of the invention said binding motif comprises an A-G nucleotide base rich loop motif separated by 3 to 5 nucleotide base pairs from a bulge region containing A and/or G nucleotide base[s].

In a preferred embodiment of the invention said stem domain comprises between 3 and 5 nucleotide base pairs, followed by a bulge region that preferentially contains A and G nucleotide bases.

In a preferred embodiment of the invention said nucleic acid based anti-viral agent comprises or consists of a nucleotide sequence set forth in the group: SEQ ID NO: 142, 143 or 144.

In a preferred embodiment of the invention said human virus is hepatitis C virus [HCV]

In a preferred embodiment of the invention said nucleic acid based anti-viral agent comprises:
i) a nucleic acid loop domain comprising 5 to 11 nucleotide bases comprising a G-rich nucleotide binding motif, preferentially containing the nucleotide bases GGG and a G and/or A nucleotide base at the start and/or end of the loop domain, for one or more HCV capsid assembly domains in a HCV capsid protein; and
ii) a nucleic acid stem domain wherein the stem domain is 14 to 23 nucleotides in length which over all or part of its length forms a double-stranded region by intramolecular complementary base pairing, wherein said anti-viral agent inhibits the formation of the HCV capsid.

In a preferred embodiment of the invention said binding motif comprises a G-rich nucleotide base motif; preferably GGG, and an A and/or G nucleotide base at the start and/or end of the loop portion.

In a preferred embodiment of the invention said nucleic acid based anti-viral agent comprises or consists of a nucleotide sequence set forth in the group: 184, 185, 186, 187, 188, 189, 190 or 191.

In a preferred embodiment of the invention said human virus is human parechovirus (HPeV).

In a preferred embodiment of the invention said nucleic acid based anti-viral agent comprises:
i) a nucleic acid loop domain comprising 4 to 6 nucleotide bases comprising a binding motif for one or more parechoviral capsid assembly domains in a parechoviral capsid protein; and
ii) a nucleic acid stem domain I stem domain comprises 13 to 35 nucleotides which over all or part of its length forms a double-stranded region by intramolecular complementary base pairing, wherein said anti-viral agent inhibits the formation of the parechoviral capsid.

In a preferred embodiment of the invention said binding motif comprises a poly-U nucleotide base motif with a single purine, preferably a G nucleotide base

In a preferred embodiment of the invention said stem domain comprises between 2 and 5 base pairs adjacent to a bulge region which is preferentially pyrimidine rich.

In a preferred embodiment of the invention said nucleic acid based anti-viral agent comprises or consists of a nucleotide sequence set forth in the group: SEQ ID NO: 1, 2, 3, 4, 5, 6, 7, 8, 9, 10, 11, 12, 13 or 14.

In a preferred embodiment of the invention said nucleic acid based anti-viral agent comprises or consists of a nucleotide sequence set forth in the group: SEQ ID NO: 578, 579, 580, 581, 582, 583, 584, 585, 586, 587, 588, 589, 590, 591, 592, 593, 594, 595, 596, 597, 598, 599, 600 or 601.

In a further embodiment of the invention said human virus is human immune deficiency virus [HIV].

In a preferred embodiment of the invention said nucleic acid based anti-viral agent comprises:
i) a nucleic acid loop domain comprising 6 to 8 nucleotide bases comprising one or two of the binding motifs comprising at least one A nucleotide base for one or more Human Immunodeficiency Virus [HIV] capsid assembly domains in a HIV capsid protein; and
ii) a nucleic acid stem domain wherein the stem domain is 4, 5, 6, 7 or 8 nucleotides in length which over all or part of its length forms a double-stranded region by intramolecular complementary base pairing, wherein said anti-viral agent inhibits the formation of the HIV capsid.

In a preferred embodiment of the invention said binding motif comprises a nucleic acid loop with one or two of the nucleotide base motifs selected from the group consisting of: [AAX . . . X], [X . . . XAA], [CAX . . . X], [X . . . XCA], [ACX . . . X], [X . . . XAC] wherein X is any nucleotide base and further wherein the nucleotide bases AA, CA, or AC is separated by one or more nucleotide bases, preferably separated by 1, 2 or 3 nucleotide bases.

In a preferred embodiment of the invention said nucleic acid based anti-viral agent comprises or consists of a nucleotide sequence as set forth in the group: SEQ ID NO: 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, or 53.

In a further preferred embodiment of the invention said nucleic acid based anti-viral agent comprises or consists of a nucleotide sequence as set forth in the group: SEQ ID NO: 573, 574, 575, 576 or 577.

In an alternative preferred embodiment of the invention said RNA virus is a plant RNA virus.

In a preferred embodiment of the invention said plant virus is Turnip Crinkle Virus.

In a preferred embodiment of the invention said nucleic acid based anti-viral agent comprises:
i) a nucleic acid loop domain comprising 7 to 12 nucleotide bases comprising a nucleotide binding motif for one or more Turnip Crinkle Virus [TCV] capsid assembly domains in a TCV capsid protein; and
ii) a nucleic acid stem domain wherein the stem domain is 24 to 32 nucleotide bases in length which over all or part of its length forms a double-stranded region by intramolecular complementary base pairing, wherein said anti-viral agent inhibits the formation of the TCV capsid.

In a preferred embodiment of the invention said nucleotide binding motif comprises a purine rich binding motif; preferably said motif comprises the nucleotide bases GGG or AAA.

In a preferred embodiment of the invention said stem domain comprises at least one purine rich bulge of three or more nucleotide bases.

In a preferred embodiment of the invention said nucleic acid based anti-viral agent comprises or consists of a nucleotide sequence set forth in the group: SEQ ID NO: 54, 55, 56, 57, 58, 59, 60, 61, 62, 63, 64, 65, 66, 67, 68, or 69.

In a preferred embodiment of the invention said nucleic acid based anti-viral agent comprises or consists of a nucleotide sequence set forth in the group 472, 473, 474 or 475.

In a preferred embodiment of the invention said plant virus is Cowpea Chlorotic Mottle Virus 1, 2 or 3.

In a preferred embodiment of the invention said nucleic acid based anti-viral agent comprises:
i) a nucleic acid loop domain comprising 4 to 8 nucleotide bases comprising a binding motif with at least one U nucleotide base for one or more Cowpea Chlorotic Mottle Virus 1 [CCMV1] capsid assembly domains in a CCMV1 capsid protein; and
ii) a nucleic acid stem domain wherein the stem domain is 8 to 31 nucleotide bases in length which over all or part of its length forms a double-stranded region by intramolecular complementary base pairing, wherein said anti-viral agent inhibits the formation of the CCMV1 capsid.

In a preferred embodiment of the invention said binding motif comprises the sequence UUXX or XXUU wherein X is any nucleotide base; preferably said motif comprises the sequence UUXA.

In a preferred embodiment of the invention said nucleic acid based anti-viral agent comprises or consists of a nucleotide sequence set forth in the group: 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369 or 370.

In a preferred embodiment of the invention said nucleic acid based anti-viral agent comprises:
i) a nucleic acid loop domain comprising 4 to 8 nucleotide bases comprising a binding motif comprising at least one U nucleotide base for one or more Cowpea Chlorotic Mottle Virus 2 [CCMV2] capsid assembly domains in a CCMV2 capsid protein; and
ii) a nucleic acid stem domain wherein the stem domain is 8 to 32 nucleotides in length which over all or part of its length forms a double-stranded region by intramolecular complementary base pairing, wherein said anti-viral agent inhibits the formation of the CCMV2 capsid.

In a preferred embodiment of the invention said binding motif comprises the sequence UUXX or XXUU wherein X is any nucleotide base; preferably the sequence UUXA.

In a preferred embodiment of the invention said nucleic acid based anti-viral agent comprises or consists of a nucleotide sequence set forth in the group: 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, or 429.

In a preferred embodiment of the invention said nucleic acid based anti-viral agent comprises:
i) a nucleic acid loop domain comprising 4 to 8 nucleotide bases comprising a binding motif comprising at least one U nucleotide base for one or more Cowpea Chlorotic Mottle Virus 3 [CCMV3] capsid assembly domains in a CCMV3 capsid protein; and
ii) a nucleic acid stem domain wherein the stem domain is 8 to 35 nucleotides in length which over all or part of its length forms a double-stranded region by intramolecular complementary base pairing, wherein said anti-viral agent inhibits the formation of the CCMV3 capsid.

In a preferred embodiment of the invention said binding motif comprises the sequence the sequence UUXX or XXUU wherein X is any nucleotide base; preferably the sequence UUXA.

In a preferred embodiment of the invention said nucleic acid based anti-viral agent comprises or consists of a nucleotide sequence set forth in the group: 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470 or 471.

In a preferred embodiment of the invention said nucleic acid based anti-viral agent comprises or consists of a nucleotide sequence set forth in the group: SEQ ID NO: 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113.

In a preferred embodiment of the invention said plant virus is Brome Mosaic Virus 1, 2, or 3.

In a preferred embodiment of the invention said nucleic acid based anti-viral agent comprises:
i) a nucleic acid loop domain comprising 4 to 8 nucleotide bases comprising a binding motif comprising at least one U nucleotide base for one or more Brome Mosaic Virus 1 [BMV1] capsid assembly domains in a BMV1 capsid protein; and
ii) a nucleic acid stem domain wherein the stem domain is 9 to 34 nucleotides in length which over all or part of its length forms a double-stranded region by intramolecular complementary base pairing, wherein said anti-viral agent inhibits the formation of the BMV1 capsid.

In a preferred embodiment of the invention said binding motif comprises the sequence UUXX or XXUU wherein X is any nucleotide base; preferably the sequence UUXA or UUXC.

In a preferred embodiment of the invention said nucleic acid based anti-viral agent comprises or consists of a nucleotide sequence set forth in the group: 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182 or 183.

In a preferred embodiment of the invention said nucleic acid based anti-viral agent comprises:
i) a nucleic acid loop domain comprising 4 to 8 nucleotide bases comprising a binding motif comprising at least one U nucleotide base for one or more Brome Mosaic Virus 2 [BMV2] capsid assembly domains in a BMV2 capsid protein; and
ii) a nucleic acid stem domain wherein the stem domain is 8 to 35 nucleotides in length which over all or part of its length forms a double-stranded region by intramolecular complementary base pairing, wherein said anti-viral agent inhibits the formation of the BMV2 capsid.

In a preferred embodiment of the invention said nucleic acid based anti-viral agent comprises or consists of a nucleotide sequence set forth in the group: 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255 or 256,

In a preferred embodiment of the invention said nucleic acid based anti-viral agent comprises:
i) a nucleic acid loop domain comprising 4 to 8 nucleotide bases comprising a binding motif comprising at least one U nucleotide base for one or more Brome Mosaic Virus 3 [BMV3] capsid assembly domains in a BMV3 capsid protein; and
ii) a nucleic acid stem domain wherein the stem domain is 9 to 38 nucleotides in length which over all or part of its length forms a double-stranded region by intramolecular complementary base pairing, wherein said anti-viral agent inhibits the formation of the BMV3 capsid.

In a preferred embodiment of the invention said binding motif comprises the sequence UUXX or XXUU wherein X is any nucleotide base; preferably said sequence is UUXA or UUXC.

In a preferred embodiment of the invention said nucleic acid based anti-viral agent comprises or consists of a nucleotide sequence set forth in the group: 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294 or 295.

In a preferred embodiment of the invention said nucleic acid based anti-viral agent comprises or consists of a nucleotide sequence set forth in the group: SEQ ID NO: 114, 115, 116, 117, 118, 119, 120, 121, 122, 123, 124, 125, 126, 127, 128, 129, 130, 131, 132, 133, 134, or 135.

In a preferred embodiment of the invention said nucleic acid based anti-viral agent comprises:
i a nucleic acid loop domain comprising 4 to 6 nucleotide bases comprising a binding motif comprising at least one A nucleotide base for one or more Satellite Tobacco Necrosis Virus 1 [STNV-1], capsid assembly domains in an STNV-1 capsid protein; and
ii a nucleic acid stem domain wherein the stem domain is 4 to 26 nucleotides in length which over all or part of its length forms a double-stranded region by intramolecular complementary base pairing, wherein said anti-viral agent inhibits the formation of the STNV 1 capsid.

In a preferred embodiment of the invention said nucleic acid based anti-viral agent comprises:
i a nucleic acid loop domain comprising 4 to 6 nucleotide bases comprising a binding motif comprising at least one A nucleotide base for one or more Satellite Tobacco Necrosis Virus 2 [STNV-2] capsid assembly domains in an STNV-2 capsid protein; and
ii a nucleic acid stem domain wherein the stem domain is 4 to 26 nucleotides in length which over all or part of its length forms a double-stranded region by intramolecular complementary base pairing, wherein said anti-viral agent inhibits the formation of the STNV-2 capsid.

In a preferred embodiment of the invention said nucleic acid based anti-viral agent comprises:
i a nucleic acid loop domain comprising 4 to 6 nucleotide bases comprising a binding motif comprising at least one A nucleotide base for one or more Satellite Tobacco Necrosis Virus c [STNV-c] capsid assembly domains in an STNV-c capsid protein; and
ii a nucleic acid stem domain wherein the stem domain is 4 to 26 nucleotides in length which over all or part of its length forms a double-stranded region by intramolecular complementary base pairing, wherein said anti-viral agent inhibits the formation of the STNV-c capsid.

In a preferred embodiment of the invention said binding motif comprises the motif selected from the group consisting of: [AX . . . XA] or [XAX . . . XA] or [AX . . . XAX] wherein X is any nucleotide base and further wherein each A nucleotide base is separated by at least one nucleotide base; preferably 1, 2 or 3 nucleotide bases

In a preferred embodiment of the invention said nucleic acid based anti-viral agent comprises or consists of a nucleotide sequence set forth in the group: SEQ ID NO: 476, 477, 478, 479, 480, 481, 482, 483, 484, 485, 486, 487, 488, 489, 490, 491, 492, 493, 494, 495, 496, 497, 498, 499, 500, 501, 502, 503, 504 or 505.

In a preferred embodiment of the invention said nucleic acid based anti-viral agent comprises or consists of a nucleotide sequence set forth in the group: SEQ ID NO: 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536 or 537.

In a preferred embodiment of the invention said nucleic acid based anti-viral agent comprises or consists of a nucleotide sequence set forth in the group: SEQ ID NO: 538, 539, 540, 541, 542, 543, 544, 545, 546, 547, 548, 549, 550, 551, 552, 553, 554, 556, 557, 558, 559, 560, 561, 562, 563, 564, 565, 566, 567, 568, 569, 570, 571 or 572.

In a preferred embodiment of the invention said nucleic acid based agent comprises modified nucleotides.

The term “modified” as used herein describes a nucleic acid molecule in which:

i) at least two of its nucleotides are covalently linked via a synthetic internucleotide linkage (i.e., a linkage other than a phosphodiester linkage between the 5′ end of one nucleotide and the 3′ end of another nucleotide). Alternatively or preferably said linkage may be the 5′ end of one nucleotide linked to the 5′ end of another nucleotide or the 3′ end of one nucleotide with the 3′ end of another nucleotide; and/or
ii) a chemical group, such as cholesterol, not normally associated with nucleic acids has been covalently attached to the single-stranded nucleic acid.
iii) Preferred synthetic internucleotide linkages are phosphorothioates, alkylphosphonates, phosphorodithioates, phosphate esters, alkylphosphonothioates, phosphoramidates, carbamates, phosphate triesters, acetamidates, peptides, and carboxymethyl esters.

The term “modified” also encompasses nucleotides with a covalently modified base and/or sugar. For example, modified nucleotides include nucleotides having sugars which are covalently attached to low molecular weight organic groups other than a hydroxyl group at the 3′ position and other than a phosphate group at the 5′ position. Thus modified nucleotides may also include 2′ substituted sugars such as 2′-O-methyl-; 2-O-alkyl; 2-O-allyl; 2′-S-alkyl; 2′-S-allyl; 2′-fluoro-; 2′-halo or 2; azido-ribose, carbocyclic sugar analogues a-anomeric sugars; epimeric sugars such as arabinose, xyloses or lyxoses, pyranose sugars, furanose sugars, and sedoheptulose.

Modified nucleotides are known in the art and include alkylated purines and/or pyrimidines; acylated purines and/or pyrimidines; or other heterocycles. These classes of pyrimidines and purines are known in the art and include, pseudoisocytosine; N4, N4-ethanocytosine; 8-hydroxy-N6-methyladenine; 4-acetylcytosine, 5-(carboxyhydroxylmethyl) uracil; 5-fluorouracil; 5-bromouracil; 5-carboxymethylaminomethyl-2-thiouracil; 5 carboxymethylaminomethyl uracil; dihydrouracil; inosine; N6-isopentyl-adenine; I-methyladenine; 1-methylpseudouracil; 1-methylguanine; 2,2-dimethylguanine; 2-methyladenine; 2-methylguanine; 3-methylcytosine; 5-methylcytosine; N6-methyladenine; 7-methylguanine; 5-methylaminomethyl uracil; 5-methoxy amino methyl-2-thiouracil; □-D-mannosylqueosine; 5-methoxycarbonylmethyluracil; 5-methoxyuracil; 2 methylthio-N6-isopentenyladenine; uracil-5-oxyacetic acid methyl ester; psuedouracil; 2-thiocytosine; 5-methyl-2 thiouracil, 2-thiouracil; 4-thiouracil; 5-methyluracil; N-uracil-5-oxyacetic acid methylester; uracil 5-oxyacetic acid; queosine; 2-thiocytosine; 5-propyluracil; 5-propylcytosine; 5-ethyluracil; 5-ethylcytosine; 5-butyluracil; 5-pentyluracil; 5-pentylcytosine; and 2,6,-diaminopurine; methylpsuedouracil; 1-methylguanine; 1-methylcytosine. Modified double stranded nucleic acids also can include base analogs such as C-5 propyne modified bases (see Wagner et al., Nature Biotechnology 14:840-844, 1996). The use of modified nucleotides confers, amongst other properties, resistance to nuclease digestion and improved stability.

According to a further aspect of the invention there is provided an anti-viral agent according to the invention for use in the treatment of viral infections.

According to a further aspect of the invention there is a pharmaceutical composition comprising an anti-viral agent and a pharmaceutical excipient.

When administered the compositions of the present invention are administered in pharmaceutically acceptable preparations. Such preparations may routinely contain pharmaceutically acceptable concentrations of salt, buffering agents, preservatives, compatible carriers and supplementary therapeutic agents.

The compositions of the invention can be administered by any conventional route, including injection or by gradual infusion over time. The administration may, for example, be oral, intravenous, intraperitoneal, intramuscular, intracavity, subcutaneous, transdermal or trans-epithelial. The compositions of the invention are administered in effective amounts. An “effective amount” is that amount of a composition that alone, or together with further doses, produces the desired response. In the case of treating a particular viral disease the desired response is inhibiting or reversing the progression of the disease. This may involve only slowing the progression of the disease temporarily to enable the host's natural antiviral defences to clear the infection and ideally reversing disease phenotype. This can be monitored by routine methods.

Such amounts will depend, of course, on the particular condition being treated, the severity of the condition, the individual patient parameters including age, physical condition, size and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

The pharmaceutical compositions used in the foregoing methods preferably are sterile and contain an effective amount of agent according to the invention for producing the desired response in a unit of weight or volume suitable for administration to a patient.

The doses of the agent according to the invention administered to a subject can be chosen in accordance with different parameters, in particular in accordance with the mode of administration used and the state of the subject. Other factors include the desired period of treatment. In the event that a response in a subject is insufficient at the initial doses applied, higher doses (or effectively higher doses by a different, more localized delivery route) may be employed to the extent that patient tolerance permits.

In general, doses of agent of between 1 nM-1 μM generally will be formulated and administered according to standard procedures. Preferably doses can range from 1 nM-500 nM, 5 nM-200 nM, and 10 nM-100 nM. Other protocols for the administration of compositions will be known to one of ordinary skill in the art, in which the dose amount, schedule of injections, sites of injections, mode of administration and the like vary from the foregoing. The administration of compositions to mammals other than humans, (e.g. for testing purposes or veterinary therapeutic purposes), is carried out under substantially the same conditions as described above. A subject, as used herein, is a mammal, preferably a human, and including a non-human primate, cow, horse, pig, sheep, goat, dog, cat or rodent.

When administered, the pharmaceutical preparations of the invention are applied in pharmaceutically-acceptable amounts and in pharmaceutically-acceptable compositions. The term “pharmaceutically acceptable” means a non-toxic material that does not interfere with the effectiveness of the biological activity of the active ingredients. Such preparations may routinely contain salts, buffering agents, preservatives, compatible carriers, and optionally other therapeutic agents used in the treatment of viral disease. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

Compositions may be combined, if desired, with a pharmaceutically-acceptable carrier. The term “pharmaceutically-acceptable carrier” as used herein means one or more compatible solid or liquid fillers, diluents or encapsulating substances which are suitable for administration into a human. The term “carrier” in this context denotes an organic or inorganic ingredient, natural or synthetic, with which the active ingredient is combined to facilitate the application, (e.g. liposome or immuno-liposome). The components of the pharmaceutical compositions also are capable of being co-mingled with the molecules of the present invention, and with each other, in a manner such that there is no interaction which would substantially impair the desired pharmaceutical efficacy.

The pharmaceutical compositions may contain suitable buffering agents, including: acetic acid in a salt; citric acid in a salt; boric acid in a salt; and phosphoric acid in a salt. The pharmaceutical compositions also may contain, optionally, suitable preservatives, such as: benzalkonium chloride; chlorobutanol; parabens and thimerosal.

The pharmaceutical compositions may conveniently be presented in unit dosage form and may be prepared by any of the methods well-known in the art of pharmacy. All methods include the step of bringing the active agent into association with a carrier which constitutes one or more accessory ingredients. In general, the compositions are prepared by uniformly and intimately bringing the active compound into association with a liquid carrier, a finely divided solid carrier, or both, and then, if necessary, shaping the product.

Compositions suitable for oral administration may be presented as discrete units, such as capsules, tablets, lozenges, each containing a predetermined amount of the active compound. Other compositions include suspensions in aqueous liquids or non-aqueous liquids such as syrup, elixir or an emulsion or as a gel. Compositions may be administered as aerosols and inhaled.

Compositions suitable for parenteral administration conveniently comprise a sterile aqueous or non-aqueous preparation of agent, which is preferably isotonic with the blood of the recipient. This preparation may be formulated according to known methods using suitable dispersing or wetting agents and suspending agents. The sterile injectable preparation also may be a sterile injectable solution or suspension in a non-toxic parenterally-acceptable dilutent or solvent, for example, as a solution in 1, 3-butane diol. Among the acceptable solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid may be used in the preparation of injectable. Carrier formulation suitable for oral, subcutaneous, intravenous, intramuscular, etc. administrations can be found in Remington's Pharmaceutical Sciences, Mack Publishing Co., Easton, Pa.

According to a further aspect of the invention there is provided a combined pharmaceutical composition comprising an agent according to the invention and one or more additional anti-viral agents different from said agent according to the invention.

In a preferred embodiment of the invention the additional anti-viral agent is an anti-retroviral agent.

Anti-viral agents are known in the art and include by example Amantadine, deoxythymidine, zidovudine, stavudine, didanosine, zalcitabine, abacavir, lamivudine, emtricitabine, tenofovir, maraviroc, efuvirtide, nevirapine, delavirdine, efavirenz, rilpivirine, Elvitegravir, Lopinavir, Indinavir, Nelfinavir, Amprenavir, Ritonavir, Bevirimat and Vivecon or combinations thereof.

Anti-viral agents also include by example: ACH-3102, Arbidol, Boceprevir, Daclatasvir, Faldaprevir, Fluvir, Ledipasvir, Moroxydine, Pleconaril, PSI-6130, Ribavirin, Rimantadine, Setrobuvir, Simeprevir, Sofosbuvir, Taribavirin and Telaprevir.

According to a further aspect of the invention the pharmaceutical composition is adapted to be delivered as an aerosol.

According to a further aspect of the invention there is provided an inhaler comprising a pharmaceutical composition according to the invention.

According to a further aspect of the invention there is provided an anti-viral agent according to the invention for use as a plant protection product in preventing or treating plant viral infections.

In a preferred embodiment of the invention said anti-viral agent is provided in a plant expression vector adapted for expression in a plant cell.

By “promoter” is meant a nucleotide sequence upstream from the transcriptional initiation site and which contains all the regulatory regions required for transcription. Suitable promoters include constitutive, tissue-specific, inducible, developmental or other promoters for expression in plant cells comprised in plants depending on design. Such promoters include viral, fungal, bacterial, animal and plant-derived promoters capable of functioning in plant cells.

Constitutive promoters include, for example CaMV 35S promoter (Odell et al. (1985) Nature 313, 9810-812); rice actin (McElroy et al. (1990) Plant Cell 2: 163-171); ubiquitin (Christian et al. (1989) Plant Mol. Biol. 18 (675-689); pEMU (Last et al. (1991) Theor Appl. Genet. 81: 581-588); MAS (Velten et al. (1984) EMBO J. 3. 2723-2730); ALS promoter (U.S. Application Ser. No. 08/409,297), and the like. Other constitutive promoters include those in U.S. Pat. Nos. 5,608,149; 5,608,144; 5,604,121; 5,569,597; 5,466,785; 5,399,680, 5,268,463; and 5,608,142, each of which is incorporated by reference.

Chemical-regulated promoters can be used to modulate the expression of a gene in a plant through the application of an exogenous chemical regulator. Depending upon the objective, the promoter may be a chemical-inducible promoter, where application of the chemical induces gene expression, or a chemical-repressible promoter, where application of the chemical represses gene expression. Chemical-inducible promoters are known in the art and include, but are not limited to, the maize ln2-2 promoter, which is activated by benzenesulfonamide herbicide safeners, the maize GST promoter, which is activated by hydrophobic electrophilic compounds that are used as pre-emergent herbicides, and the tobacco PR-1a promoter, which is activated by salicylic acid. Other chemical-regulated promoters of interest include steroid-responsive promoters (see, for example, the glucocorticoid-inducible promoter in Schena et al. (1991) Proc. Natl. Acad. Sci. USA 88: 10421-10425, and McNellis et al. (1998) Plant J. 14(2): 247-257) and tetracycline-inducible and tetracycline-repressible promoters (see, for example, Gatz et al. (1991) Mol. Gen. Genet. 227: 229-237, and U.S. Pat. Nos. 5,814,618 and 5,789,156, herein incorporated by reference).

Where enhanced expression in particular tissues is desired, tissue-specific promoters can be utilised. Tissue-specific promoters include those described by Yamamoto et al. (1997) Plant J. 12(2): 255-265; Kawamata et al. (1997) Plant Cell Physiol. 38(7): 792-803; Hansen et al. (1997) Mol. Gen. Genet. 254(3): 337-343; Russell et al. (1997) Transgenic Res. 6(2): 157-168; Rinehart et al. (1996) Plant Physiol. 112(3): 1331-1341; Van Camp et al. (1996) Plant Physiol. 112(2): 525-535; Canevascni et al. (1996) Plant Physiol. 112(2): 513-524; Yamamoto et al. (1994) Plant Cell Physiol. 35(5): 773-778; Lam (1994) Results Probl. Cell Differ. 20: 181-196; Orozco et al. (1993) Plant Mol. Biol. 23(6): 1129-1138; Mutsuoka et al. (1993) Proc. Natl. Acad. Sci. USA 90 (20): 9586-9590; and Guevara-Garcia et al (1993) Plant J. 4(3): 495-50.

“Operably linked” means joined as part of the same nucleic acid molecule, suitably positioned and oriented for transcription to be initiated from the promoter. DNA operably linked to a promoter is “under transcriptional initiation regulation” of the promoter. In a preferred aspect, the promoter is a tissue specific promoter, an inducible promoter or a developmentally regulated promoter.

Particularly of interest in the present context are nucleic acid constructs which operate as plant vectors. Specific procedures and vectors previously used with wide success in plants are described by Guerineau and Mullineaux (1993) (Plant transformation and expression vectors. In: Plant Molecular Biology Labfax (Croy RRD ed) Oxford, BIOS Scientific Publishers, pp 121-148). Suitable vectors may include plant viral-derived vectors (see e.g. EP194809). If desired, selectable genetic markers may be included in the construct, such as those that confer selectable phenotypes such as resistance to herbicides (e.g. kanamycin, hygromycin, phosphinotricin, chlorsulfuron, methotrexate, gentamycin, spectinomycin, imidazolinones and glyphosate).

According to a further aspect of the invention there is provided a transgenic plant cell transfected with an expression vector according to the invention.

According to a further aspect of the invention there is provided a plant comprising a plant cell according to the invention.

According to a further aspect of the invention there is provided a method to screen for anti-viral agents that bind to one or more packaging signals and/or one or more viral capsid proteins comprising the steps:
i) providing a preparation comprising a combinatorial library of small molecular weight compounds and contacting said library with a preparation comprising:
   a. a viral capsid protein or part thereof; or
   b. a viral packaging signal;
ii) providing conditions sufficient to allow the binding of one or more compounds to either said viral capsid protein or viral packaging signal;
iii) selecting candidate agents that associate or bind either the viral capsid protein or viral packaging signal; and
iv) testing the activity of a selected compound for anti-viral activity.

In a preferred method of the invention said viral packaging signal is derived from human parecho virus and comprises the nucleotide sequence selected from the group: SEQ 1, 2, 3, 4, 5, 6, 7, 8, 9, 10, 11, 12, 13 or 14.

In a preferred method of the invention said viral packaging signal is derived from human parecho virus and comprises the nucleotide sequence selected from the group: SEQ ID NO: 578, 579, 580, 581, 582, 583, 584, 585, 586, 587, 588, 589, 590, 591, 592, 593, 594, 595, 596, 597, 598, 599, 600 or 601.

In a preferred method of the invention said viral capsid protein is derived from human parecho virus and comprises the capsid protein SEQ ID NO: 137.

In a preferred method of the invention said viral packaging signal is derived from HIV selected from the group consisting of: SEQ ID NO: SEQ ID NO: 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, or 53.

In a further preferred method of the invention said viral packaging signal is derived from HIV selected from the group consisting of: SEQ ID NO: 573, 574, 575, 576 or 577.

In a further alternative preferred method of the invention said viral capsid protein is derived from HIV and comprises the capsid protein SEQ ID NO: 140 or 141.

In a preferred method of the invention said viral packaging signal is derived from Turnip Crinkle Virus comprises the nucleotide sequence selected from the group: SEQ ID NO: 54, 55, 56, 57, 58, 59, 60, 61, 62, 63, 64, 65, 66, 67, 68 or 69.

In a further preferred method of the invention said viral packaging signal is derived from Turnip Crinkle Virus comprises the nucleotide sequence selected from the group: SEQ ID NO: 472, 473, 474 or 475.

In a preferred method of the invention said viral capsid protein is derived from Turnip Crinkle Virus and comprises the capsid protein SEQ ID NO: 136.

In a preferred method of the invention said viral packaging signal is derived from Cowpea Chlorotic Mottle Virus selected from the group consisting of: SEQ ID NO: 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112 or 113.

In an alternative preferred method of the invention said viral packaging signal is derived from Cowpea Chlorotic Mottle Virus selected from the group consisting of: SEQ ID NO:296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470 or 471.

In an alternative method embodiment of the invention said viral capsid protein is derived from Cowpea Chlorotic Mottle Virus and comprises the capsid protein SEQ ID NO: 138.

In a preferred method of the invention said viral packaging signal is derived from Brome Mosaic Virus selected from the group consisting of: SEQ ID NO: 114, 115, 116, 117, 118, 119, 120, 121, 122, 123, 124, 125, 126, 127, 128, 129, 130, 131, 132, 133, 134 or 135.

In a preferred method of the invention said viral packaging signal is derived from Brome Mosaic Virus selected from the group consisting of: SEQ ID NO: 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294 or 295.

In an alternative method of the invention said viral capsid protein is derived from Brome Mosaic Virus and comprises the capsid protein SEQ ID NO: 139.

In a preferred method of the invention said viral packaging signal is derived from STNV-1 selected from the group consisting of: SEQ ID NO: 476, 477, 478, 479, 480, 481, 482, 483, 484, 485, 486, 487, 488, 489, 490, 491, 492, 493, 494, 495, 496, 497, 498, 499, 500, 501, 502, 503, 504 or 505.

In a preferred method of the invention said viral packaging signal is derived from STNV-2 selected from the group consisting of: SEQ ID NO: 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536 or 537.

In a preferred method of the invention said viral packaging signal is derived from STNV-c selected from the group consisting of: SEQ ID NO: 538, 539, 540, 541, 542, 543, 544, 545, 546, 547, 548, 549, 550, 551, 552, or 553.

In a preferred method of the invention said viral capsid protein is derived from STNV-1.

In a preferred method of the invention said viral capsid protein is derived from STNV-2.

In a preferred method of the invention said viral capsid protein is derived from STNV-c.

According to a further aspect of the invention there is provided a modelling method to determine the association of an anti-viral agent with a viral capsid protein or a viral packaging signal comprising the steps:
i) providing computational means to perform a fitting operation between a candidate agent and
   a) a viral capsid protein or part thereof; or
   b) a viral packaging signal; and
ii) analysing the results of said fitting operation to quantify the association between the agent and the viral capsid protein or part thereof or the viral packaging signal.

In the computational design protein ligands demand various computational analyses which are necessary to determine whether a molecule is sufficiently similar to the target moiety or structure. Such analyses may be carried out in current software applications, such as the Molecular Similarity application of QUANTA (Molecular Simulations Inc., Waltham, Mass.) version 3.3, and as described in the accompanying User's Guide, Volume 3 pages. 134-135. The Molecular Similarity application permits comparisons between different structures, different conformations of the same structure, and different parts of the same structure. Each structure is identified by a name. One structure is identified as the target (i.e., the fixed structure); all remaining structures are working structures (i.e., moving structures). When a rigid fitting method is used, the working structure is translated and rotated to obtain an optimum fit with the target structure.

The person skilled in the art may use one of several methods to screen chemical entities or fragments for their ability to associate with a target. The screening process may begin by visual inspection of the target on the computer screen, generated from a machine-readable storage medium. Selected fragments or chemical entities may then be positioned in a variety of orientations, or docked, within that binding pocket. Docking may be accomplished using software such as Quanta and Sybyl, followed by energy minimization and molecular dynamics with standard molecular mechanics force fields, such as CHARMM and AMBER.

Specialized computer programs may also assist in the process of selecting fragments or chemical entities. These include: GRID (P. J. Goodford, “A Computational Procedure for Determining Energetically Favorable Binding Sites on Biologically Important Macromolecules”, J. Med. Chem., 28, pp. 849-857 (1985)). GRID is available from Oxford University, Oxford, UK; MCSS (A. Miranker et al., “Functionality Maps of Binding Sites: A Multiple Copy Simultaneous Search Method.” Proteins: Structure, Function and Genetics, 11, pp. 29-34 (1991)). MCSS is available from Molecular Simulations, Burlington, Mass.; AUTODOCK (D. S. Goodsell et al., “Automated Docking of Substrates to Proteins by Simulated Annealing”, Proteins: Structure, Function, and Genetics, 8, pp. 195-202 (1990)). AUTODOCK is available from Scripps Research Institute, La Jolla, Calif.; DOCK (I. D. Kuntz et al., “A Geometric Approach to Macromolecule-Ligand Interactions”, J. Mol. Biol., 161, pp. 269-288 (1982)). DOCK is available from University of California, San Francisco, Calif. Each of these citations is incorporated by reference.

Once suitable chemical entities have been selected, they can be assembled into a single compound or complex. This would be followed by manual model building using software such as Quanta or Sybyl. Useful programs to aid the person skilled in the art in connecting the individual chemical entities or fragments include: CAVEAT (P. A. Bartlett et al, “CAVEAT: A Program to Facilitate the Structure-Derived Design of Biologically Active Molecules”. In: “Molecular Recognition in Chemical and Biological Problems”, Special Pub., Royal Chem. Soc., 78, pp. 182-196 (1989)). CAVEAT is available from the University of California, Berkeley, Calif., 3D Database systems such as MACCS-3D (MDL Information Systems, San Leandro, Calif.). This is reviewed in Y. C. Martin, “3D Database Searching in Drug Design”, J. Med. Chem., 35, pp. 2145-2154 (1992); and HOOK (available from Molecular Simulations, Burlington, Mass.). These citations are incorporated by reference.

As the skilled reader will already know instead of proceeding to build a ligand for the target in a step-wise fashion, target-binding compounds may be designed as a whole or de novo. These methods include: LUDI (H.-J. Bohm, “The Computer Program LUDI: A New Method for the De Novo Design of Enzyme Inhibitors”, J. Comp. Aid. Molec. Design, 6, pp. 61-78 (1992)). LUDI is available from Biosym Technologies, San Diego, Calif.; LEGEND (Y. Nishibata et al., Tetrahedron, 47, p. 8985 (1991)). LEGEND is available from Molecular Simulations, Burlington, Mass.; LeapFrog (available from Tripos Associates, St. Louis, Mo.), each of which is incorporated by reference. Other molecular modelling techniques may also be employed, see, e.g., N. C. Cohen et al., “Molecular Modeling Software and Methods for Medicinal Chemistry, J. Med. Chem., 33, pp. 883-894 (1990). See also, M. A. Navia et al., “The Use of Structural Information in Drug Design”, Current Opinions in Structural Biology, 2, pp. 202-210 (1992), which are incorporated by reference.

Typically, once a compound has been designed or selected by the above methods, the efficiency with which that entity binds to a target may be tested and optimized by computational evaluation. For example, an effective ligand will preferably demonstrate a relatively small difference in energy between its bound and free states (i.e., a small deformation energy of binding). Thus, the most efficient ligands should preferably be designed with deformation energy of binding of not greater than about 10 kcal/mol, preferably, not greater than 7 kcal/mol.

A ligand designed or selected as binding to a target may be further computationally optimized so that in its bound state it would preferably lack repulsive electrostatic interaction with the target enzyme. Such non-complementary (e.g., electrostatic) interactions include repulsive charge-charge, dipole-dipole and charge-dipole interactions. Specifically, the sum of all electrostatic interactions between the inhibitor or other ligand and the target, when the inhibitor is bound to the target, preferably make a neutral or favourable contribution to the enthalpy of binding.

Specific computer software is available in the art to evaluate compound deformation energy and electrostatic interaction. Examples of programs designed for such uses include: Gaussian 92, revision C (M. J. Frisch, Gaussian, Inc., Pittsburgh, Pa. COPYRGT. 1992); AMBER, version 4.0 (P. A. Kollman, University of California at San Francisco, .COPYRGT. 1994); QUANTA/CHARMM (Molecular Simulations, Inc., Burlington, Mass. COPYRGT. 1994); and Insight II/Discover (Biosysm Technologies Inc., San Diego, Calif. COPYRGT. 1994). These programs may be implemented, for instance, using a Silicon Graphics workstation, IRIS 4D/35 or IBM RISC/6000 workstation model 550. Other hardware systems and software packages will be known to those skilled in the art.

Once the ligand has been optimally selected or designed, as described above, substitutions may then be made in some of its atoms or side groups in order to improve or modify its binding properties. Generally, initial substitutions are conservative, i.e., the replacement group will have approximately the same size, shape, hydrophobicity and charge as the original group.

Another approach is the computational screening of small molecule data bases for chemical entities or compounds that can bind in whole, or in part, to a target. In this screening, the quality of fit of such entities to the binding site may be judged either by shape complementarity or by estimated interaction energy (E. C. Meng et al., J. Comp. Chem., 13, pp. 505-524 (1992)). The computational analysis and design of molecules, as well as software and computer systems therefore are described in U.S. Pat. No. 5,978,740 which is included herein by reference.

According to an aspect of the invention there is provided a screening method for identification of nucleic acid based agents comprising one or more nucleotide sequences comprising a binding motif for one or more capsid assembly domains in a viral capsid protein comprising the steps:
i) forming a preparation comprising a viral capsid protein and a library of nucleic acid based agents;
ii) providing conditions suitable for specifically binding a nucleic acid based agent in (i) above with one or more capsid proteins;

iii) eluting capsid bound nucleic binding agents from said capsid protein[s];
iv) amplification of the eluted nucleic acid binding agents in (iii) above;
v) repeat steps (ii) to (iv) one or more times to enrich for said nucleic acid based agent[s]; and
vi) determine the sequence of the enriched nucleic acid based agent[s].

In a preferred method of the invention the nucleic acid based agent[s] are tested for inhibition of viral capsid formation.

According to a further aspect of the invention there is provided an enriched nucleic acid based agent isolated by the method according to the invention.

According to a further aspect of the invention there is provided a method to determine one or more packaging signals in an RNA virus comprising the steps:
i) providing a nucleotide sequence of one or more nucleic acid binding agents selected by the method according to the invention;
ii) comparing the nucleotide sequence in (i) above with the genomic nucleotide sequence of an RNA virus to be assessed for the presence of a packaging signal;
iii) selecting a genomic RNA sequence based on a degree of similarity to the nucleotide sequence in (i) above; and optionally
iv) determining whether the selected genomic RNA sequence or part thereof binds the viral capsid protein of the RNA virus.

In a preferred method of the invention the selected genomic RNA sequence is correlated with the anti-viral capsid binding activity of the nucleic acid binding agent selected in (i) above thereby ranking the importance of the selected packaging signal for assembly.

Throughout the description and claims of this specification, the words “comprise” and “contain” and variations of the words, for example “comprising” and “comprises”, means “including but not limited to”, and is not intended to (and does not) exclude other moieties, additives, components, integers or steps. “Consisting essentially” means having the essential integers but including integers which do not materially affect the function of the essential integers.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

An embodiment of the invention will now be described by example only and with reference to the following figures:

### BRIEF DESCRIPTION OF THE DRAWINGS

FIG. 1A illustrates: Histogram plot of aptamer hits on the HPeV1 Harris genome sequence. The peaks represent alignments between aptamers and sequence motifs with Bernoulli scores of 12 or above. Two regions containing significant peaks within the coding region of the genome are marked by arrows and boxed, and discussed in more detail in FIG. 1B.

FIGS. 1B and 1C illustrate: Identification of packaging signals compared to a naïve unselected library. The two areas highlighted in FIG. 1A are shown in magnification, and the secondary structure of the genome fragment corresponding to the highest peak in each area is shown underneath the arrow (FIG. 1B, area 1=nucleotides 3-21 of SEQ ID NO: 584; FIG. 1C, area 2=SEQ ID NO: 7). Coincidence with the best-matching aptamer sequence is indicated via capital letters in the adjacent stem-loop.

FIG. 1D illustrates: Nucleotide variation plots across 21 different strains identify areas conserved across all strains. Nucleotide variation plots are superimposed on the analysis in FIGS. 1A-1C, demonstrating that areas identified correspond to conserved areas across different strains, as expected for motifs that have functional significance. Due to the averaging procedure (over fragments of 5 nt) a zero value indicates perfect alignment of at least contiguous nucleotides.

FIG. 1E illustrates: Alignment of aptamers from SELEX against the viral genome using Bernoulli scores. Bernouilli peaks are shown in green, compared to background signals in red;

FIGS. 1F-1H illustrate: Predicted mfold structures for the 22 packaging sequences in the human parechovirus genome 1 after analysing the Bernouilli peaks in FIG. 1F (PS1 to PS12 are shown in SEQ ID NOS: 578-589, respectively, FIG. 1G PS13-PS22 are shown in SEQ ID NOS: 590-599, respectively). PS9 with silent mutations to CUGGAAGUGUAGUAACAUUCCAG (mutated residues in bold) and PS22 to AAGACGAAUGAAACGUUCGUCUU were introduced in to cDNA copies of the viral genome. The cDNA shows a 4 log reduction in titre of productive virus compared to the WT after 24 hours (FIG. 16) and a 6 log reduction after 96 hours (FIG. 17). In addition, when cells are loaded with this mutated mRNA (the virus genome is equivalent to the mRNA) and challenged 6 hours later with wild type virus the mutant mRNA delays the onset of infection (FIG. 18). Among the different folds of similar energy returned by Mfold, we have chosen those that show the strongest similarity with the folds of the 5 most abundant aptamers returned by SELEX (cf. FIG. 1H, APT 1-APT 5 are shown in SEQ ID NOS: 10-14, respectively). These packaging signals are labelled HPeV-PS1 to HPeV-PS22 in FIGS. 1F-1G;

FIG. 2A illustrates: Alignment plots for Toy. Peaks labelled correspond to packaging signals as indicated;

FIG. 2B illustrates: Secondary structures of the TCV packaging signals corresponding to the peaks in FIG. 2A (1=SEQ ID NO: 472, 2=SEQ ID NO: 473, 3=SEQ ID NO: 474, 4=SEQ ID NO: 475);

FIG. 3A illustrates: Alignment plots for CCMV1, 2 and 3. The 5 highest peaks in CCMV1 &2, and the 4 highest peaks in CCMV 3 have been identified as putative packaging signals;

FIG. 3B illustrates: Schematic representation of the packaging signals. PS positions are indicated with reference to the gene product in each segment; the green one corresponds to the known B-Box;

n

FIG. 3C illustrates: Secondary structures of the CCMV aptamer sequences, with N indicating their frequency of occurrence in the aptamer pool (APT1=SEQ ID NO: 108, APT2=SEQ ID NO: 109, APT3=SEQ ID NO: 110, APT4=SEQ ID NO: 111, APT5=SEQ ID NO: 112, APT6=SEQ ID NO: 113);

FIGS. 3D-3E illustrate: Secondary structures of the packaging signals corresponding to the largest peaks in FIG. 3A;

FIG. 4: The secondary structures of the HIV-1 secondary packaging signals in the HxB2 strain. From left to right, top to bottom, PS1 (SEQ ID NO: 573), PS2a (SEQ ID NO: 574) and PS2b (SEQ ID NO: 574) (two different possible folds for PS2 resulting in the same loop sequence), PS3 (SEQ ID NO: 575), PS4 (SEQ ID NO: 576) and PS5 (SEQ ID NO: 577);

FIG. 5: Top shows single molecule FCS re-assembly as time-dependent or Rh distribution plots. SL1/3 are HepB PSs, epsilon is the known “assembly site” that binds polymerase; B3 is a PS for STNV-1, TEMs of assembly products are shown coded red. Bottom shows Hepatitis B reassembly in presence of PSs monitored by single molecule fluorescence correlation spectroscopy (smFCS) and Transmission Electron Microscopy (TEM);

FIG. 6A: Packaging signal of Hepatitis B virus. 1 (1722-1756) 5′-UUUGUUUAAAGACUGGGAGGAGUUGGGGGAGGAG-3 ‘ (SEQ ID NO: 142),

FIG. 6B Packaging signal 2 of Hepatitis B virus (2583-2636); 5’-GUGGGCCCUCUGACAGUUAAUGAAAAAAGGAGAUUAAAAUUAAUUAUGCCUGC-3′ (SEQ ID NO: 143),

FIG. 6C Packaging signal 3 of Hepatitis B virus (2761-2804) 5′-GGAAGGCUGGCAUUCUAUAUAAGAGAGAAACUACACGCAGCGCC-3′ (SEQ ID NO: 144);

FIG. 7A: Illustration of the PS-mediated assembly of the STNV capsid. B3 binding facilitates coat protein association and renders capsid assembly more efficient.

FIG. 7B: Natural PSs at the 5′ end of the STNV-1 genome;

FIG. 7C: PS positions in the STNV genome with reference to the coat protein gene;

FIGS. 8A-8B: Evidence that natural PSs exist, are recognised sequence-specifically, work co-operatively and that their relative positioning along the genome is vital. FIG. 8 A compares the co-operative assembly via smFCS of the 5′ fragment from the STNV genome with 5 PSs (black) vs a single PS (purple) (top). The figures in the middle and bottom show a same-sized genomic fragment with sequences of PSs flanking high affinity site mutated (blue). FIG. 8B STNV reassembly in presence of PS 1-5 with a 10 nucleotide insert either 3′, 5′ or both sides of the high affinity PS3 site monitored by single molecule fluorescence correlation spectroscopy (smFCS) plotted as a time course and a distribution plot;

FIGS. 9A-9E: CCMV1 packaging signals identified from the consensus recognition motifs described above (SEQ ID NO 296-370, from left to right and top to bottom);

FIGS. 10A-10D: CCMV2 packaging signals identified from the consensus recognition motifs described above (SEQ ID NO 371-429, from left to right and top to bottom);

FIGS. 11A-11D: CCMV3 packaging signals identified from the consensus recognition motifs described above (SEQ ID NO 430-471, from left to right and top to bottom);

FIGS. 12A-12D: BMV1 packaging signals identified from the consensus recognition motifs described above (SEQ ID NO 145-183, from left to right and top to bottom);

FIGS. 13A-13C: BMV2 packaging signals identified from the consensus recognition motifs described above (SEQ ID NO 192-256, from left to right and top to bottom);

FIGS. 14A-14B: BMV3 packaging signals identified from the consensus recognition motifs described above (SEQ ID NO 257-295, from left to right and top to bottom);

FIGS. 15A-15B: Positions of the CCMV and BMV PSs in the respective genomes;

FIG. 16: Determination of infection potential of packaging signal mutants of HPeV1. The supernatant of freeze-thawed GMK cell lysate transfected with cDNA wild type, packaging signal mutants PS3, PS9, PS11, PS19 or PS22 was added on HPeV1-sensitive HT29 cells in 10-fold serial dilution. The infectivity was recorded as the extent of cytopathic effect (CPE) for each dilution. The CPE score was as follows: 5, All cells lysed; 4, 75%-100% CPE; 3, 50%-75% CPE; 2, 25%-50% CPE; 1, 10-25% CPE; 0, No CPE. The assay was done in triplicate. The above graph shows the CPE score for 10-fold serial dilution up to 10-6 at 24 h post infection. At short times after transfection, PS22 and PS9 show significantly less virus production than PS3 or PS11. PS19 has a mild effect only evident in the third dilution;

FIG. 17: Determination of infection potential of packaging signal mutants of HPeV1. The supernatant of freeze-thawed GMK cell lysate transfected with cDNA wild type, packaging signal mutants PS3, PS9, PS11, PS19 or PS22 was added on HPeV1-sensitive HT29 cells in 10-fold serial dilution. The infectivity was recorded as the extent of cytopathic effect (CPE) for each dilution. The CPE score was as follows: 5, All cells lysed; 4, 75%-100% CPE; 3, 50%-75% CPE; 2, 25%-50% CPE; 1, 10-25% CPE; 0, No CPE. The assay was done in triplicate. The above graph shows the CPE score for 10-fold serial dilution up to 10-6 at 96 h post infection. At longer times of incubation, still there is no evident CPE formed for PS22 and PS9 is greatly reduced compared to wild type. PS19 has a much milder effect;

FIG. 18: Competitive assay between RNA of packaging signal mutants PS9 or PS22 against the wild type virion. GMK cells were transfected with PS9 or PS22 mutant RNA of the same length as the wild type genome, followed by infection with wild type virion at 6 h post transfection. At 24 h post infection, the supernatant of freeze-thawed GMK cell lysate of wild type or mutants was added on HPeV1-sensitive HT29 cells in 10-fold serial dilution. The infectivity was recorded as the extent of cytopathic effect (CPE) for each dilution. The CPE score was as follows: 5, All cells lysed; 4, 75%-100% CPE; 3, 50%-75% CPE; 2, 25%-50% CPE; 1, 10-25% CPE; 0, No CPE. The assay was done in triplicate. The above graph shows the CPE score for 10-fold serial dilution up to 10-7 at 48 h post infection. At higher dilutions on the sensitive cell line, the mutant RNAs show delayed onset of infection (readout as lower cytopathic effect compared to the untransfected cells treated with virus);

FIG. 19: Hepatitis C virus-packaging signals. Predicted structures, based upon mFold analysis of the selected RNA aptamers and comparison to the HCV genome. The packaging signals are named according to the position of the first nucleotide within the JFH1 strain of HCV (GenBank accession AB047639.1). Their conserved features include a hairpin structure (7 of the 8 possess an internal bulge) and a purine-rich terminal loop. (SL733=SEQ ID NO: 184, SL2899=SEQ ID NO: 185, SL3789=SEQ ID NO: 186, SL4629=SEQ ID NO: 187, SL4807=SEQ ID NO: 188, SL5877=SEQ ID NO: 189, SL6067=SEQ ID NO: 190, SL7580=SEQ ID NO: 191);

FIG. 20: The impact of PSs on STNV assembly. Coloured lines are; Black=5 PS construct (PS1-5); red=PS1, 2, 3, green=PS2, 3, 4; and blue=PS3, 4, 5. Shows the three PS constructs do not form capsids; this illustrates that fragments containing incomplete sets of PSs can inhibit assembly. In this case fragments carrying just 3 out of 5 PSs inhibit assembly by misdirecting the assembly intermediate to an off-assembly pathway state;

FIGS. 21A-21B: STNV-1 packaging signals identified from the consensus recognition motifs described above (SL1-SL30 are SEQ ID NOS: 476-505, respectively);

FIGS. 22A-22B: STNV-2 packaging signals identified from the consensus recognition motifs described above (SL1-SL32 are SEQ ID NOS: 506-537, respectively); and

FIGS. 23A-23B: STNV-c packaging signals identified from the consensus recognition motifs described above (SL1-SL35 are SEQ ID NOS: 538-572, respectively).

### Materials & Methods

SELEX: In Vitro Isolation of RNA Oligos with High Affinity for Viral CPs.

Initial selection libraries are described as xN, where x is the number of degenerate nucleotides (N) in a row in the library. X defines the random region and is sometimes referred to as the selected region. These libraries are prepared as dsDNA fragments synthesised by commercially. As well as the random region they encompass defined sequence regions on either side. On the 5′ side they encompass a promoter for the bacteriophage T7 RNA polymerase, allowing transcription to create the RNA library, whilst at the 3′ side they have a short fixed region to allow recovery and amplification of the aptamers that bind to the desired target.

Following completion of the SELEX process pools were amplified by a further 10 rounds of PCR to produce enough material for sequencing. The PCR product for each SELEX library was then purified using a commercial PCR DNA clean up kit to remove the excess nucleotides and enzymes. Adaptor DNA sequences needed for the Illumina MiSeq next generation sequencing machine were ligated onto the PCR products and further amplification was carried out. These libraries were then loaded on the next generation sequencing machine.

### Brome Mosaic Virus (BMV) and Cowpea Chlorotic Mosaic Virus (CCMV)

Whole virions, gifts from Prof William Gilbert at UCLA, were biotinylated using the chemical modification reagent, EZ-link biotin (Pierce) which modifies surface lysine residues. The reaction is deliberately incomplete implying that lysines are modified at random and that each protein will carry one or very few biotin labels. Modified virus particles were then dissociated by altering solution conditions, thus ensuring that only the outside of the CPs was biotinylated.

Biotinylated CPs were incubated with streptavidin beads for 1 hour and then washed with 5 mM Tris-HCl (pH 7.5) 1 M NaCl (note: all buffers contained protease inhibitor) three times (to remove excess coat protein and RNA). At this point the beads were split in half and washed three times either with RNA assembly buffer (50 mM NaCl, 10 mM KCl, 5 mM MgCl₂, 1 mM DTT, 50 mM Tris-HCl pH 7.2) or virus suspension buffer (50 mM sodium acetate, 8 mM magnesium acetate pH 4.5) to create pH 7.2 and pH 4.5 positive selection beads, respectively.

An N40 2′F RNA library (modified CTP and UTP) was used (to protect against nuclease activity) for selection. Three transcriptions of the N40 library were performed, pooled together and then split evenly between the two pH selections (this ensured both pH selections had the same starting material).

Fourteen standard rounds of SELEX were performed whereby the negative beads were bare streptavidin beads, which had been washed in the same manner as the positive beads (to remove RNA sequences that bound to streptavidin). The RNA library was incubated with negative and positive beads for 5 minutes at 37° C.

The 2^{nd} and 8^{th} rounds of selection were done as normal but before the SELEX the RNA library was exposed to 0.1 mg/mL of biotinylated capsid (this removed RNA sequences with a greater affinity either for the outside of the capsid or for the biotin linker). The capsids were then pulled out of solution using streptavidin beads. The remaining RNA was then used as normal.

The final round of selection was a standard round of SELEX but the positive beads were exposed to 0.1 mg/mL of unbiotinylated capsid (to remove RNA sequences with a greater affinity for the outside of the capsid).

### Turnip Crinkle Virus (TCV)

Whole virions, a gift from Drs George Lomonossoff & Keith Saunders at the John Innes Centre, Norwich, were biotinylated and then dissociated into high-salt/pH buffer. Biotinylated coat proteins were incubated with streptavidin beads for 1 hour and then washed with 50 mM PIPES (ph 6.5), 2 mM MgCl₂, 50 mM NaCl (note: all buffers contained protease inhibitor) three times.

An N30 RNA library was used for selection. Selection buffer was 50 mM PIPES (pH 6.5), 2 mM MgCl₂, 50 mM NaCl.

Fourteen standard rounds of SELEX were performed whereby the negative beads were bare streptavidin beads, which had been washed in the same manner as the positive beads. RNA library was incubated with negative and positive beads for 5 minutes at 37° C.

The 2^{nd} and 8^{th} rounds of selection were done as normal but before the SELEX the RNA library was exposed to 0.1 mg/mL of biotinylated capsid. The capsids were then pulled out of solution using streptavidin beads. The remaining RNA was then used as normal.

The final round of selection was a standard round of SELEX but the positive beads were exposed to 0.1 mg/mL of unbiotinylated capsid.

### Human Parechovirus 1 (HPeV 1)

Samples of HPeV1 CP as a pentamer were supplied by our collaborator, Prof Sarah Butcher from the University of Helsinki.

The virus was buffered exchanged to PBS using a 100 kDa cutoff centricon (Millipore). It was mixed with biotin (NHS-LC-LC-biotin, Pierce) at a molar ratio of 1:20 of number of lysines on the virus capsid to the biotin and kept at room temperature for 2 h. Unreacted biotin was quenched using 1M Tris-HCl, pH 8.2 and the biotinylated virus was buffer exchanged to TNM buffer (10 mM Tris-HCl pH 7.7, 150 mM NaCl and 1 mM MgCl₂) using a 100 kDa cutoff centricon (Millipore).

The biotinylated virus was heated at 56° C. for 30 min to disrupt it into pentamers and centrifuged at 92000 rpm for 10 min at room temperature in Beckman Coulter Airfuge with A-110 fixed angle rotor to pellet down undisrupted capsids. The supernatant was collected and pentamer formation was confirmed by running native 4-20% (w/v) Tris glycine gel (Biorad) with NativeMark unstained protein standards (Cat#LC0725, Life technologies). In addition, thyroglobulin (669 kDa) and β amylase (200 kDa) were used as two other reference standards. A band of the expected size for a pentamer containing all three capsid proteins was observed at ˜431 kDa.

Biotinylated coat proteins were incubated with streptavidin beads for 1 hour and then washed with 5 mM Tris-HCl (pH 7.5) 1 M NaCl (note: all buffers contained protease inhibitor) three times (to remove excess coat protein and RNA) with 10 mM Tris-HCl, pH 7.7, 150 mM NaCl. An N40 RNA library was used for selection. Selection buffer was 10 mM Tris-HCl, pH 7.7, 150 mM NaCl

Eleven standard rounds of SELEX were performed whereby the negative beads were either bare streptavidin beads or biotinylated capsid. The RNA library was incubated with negative and positive beads for 5 min at 37° C.

Negative selections were alternated at each round, i.e. round 1 used bare streptavidin beads and round 2 used biotinylated capsid.

### Methods for Characterising Packaging Signals

The large numbers of putative PSs uncovered by SELEX and bioinformatics cannot be analysed by traditional approaches. We have therefore devised a protocol for high-throughput screening. Single-stranded DNA oligos encompassing all the RNA sites to be tested, designed to incorporate flanking sites for amplification and T7 RNA polymerase transcription, are purchased, used to create dsDNA templates for in vitro transcription and the transcripts aliquoted into in vitro binding/assembly assays using fluorescently-labelled viral CPs. The CP-PS affinities will be determined initially using thermophoresis (MST), which monitors the movement of dye-labelled species in differentially heated solution. MST requires only ˜10 μL of sample, is rapid (<1 h), not destructive and cheap. Binding curves are constructed via titrations of up to 16 ligand concentrations at a time and we have shown that this yields the same Kd for the MS2 CP-TR (its highest affinity PS) interaction as stopped-flow fluorescence measurements. Surface Plasmon Resonance, stopped-flow fluorescence, isothermal titration calorimetry and single molecule fluorescence spectroscopy can all then be used for assessing the effects of drugs on the CP-PS interaction. If PS-CP interaction triggers assembly, it can be detected using fluorescence anisotropy. The structures of assembled material can then be assessed by negative-stain transmission electron microscopy (TEM) and determined by cryo-EM reconstruction. Those PSs with the highest CP affinity and favourable effects on CP assembly are then subjected to more thorough analysis including making sequence variants to determine the precise sequences/motifs required for CP binding.

Methods for Identifying Small Molecular Weight Drugs that Bind PSs or their CP Binding Sites.

PSs are most likely to encompass at least one stem-loop, the lowest level of secondary structure within RNAs. These do not have unique structures in solution but exist as ensembles of differing conformations in equilibrium with each other. Traditionally this has made isolation of specific binding ligands difficult. However, a generic method for isolation of ligands with nanomolar affinities has recently been developed iDiscovery of selective bioactive small molecules by targeting an RNA dynamic ensemble. Stelzer A C, Frank A T, Kratz J D, Swanson M D, Gonzalez-Hernandez M J, Lee J, Andricioaei I, Markovitz D M, Al-Hashimi H M. Nat Chem Biol. 2011 Jun. 26; 7(8):553-9) using NMR structure determination to define the principal conformers of the RNA and de novo drug design strategies that are routine within the Pharma industry. Similar ligands that bind to the PS binding sites on viral CPs can be designed/screened for, once the structures of the PS-CP complex are known from X-ray crystallography or NMR spectroscopy.

### Bioinformatics

For each virus all unique aptamer sequences from next generation sequencing results were aligned to available strains using the following in-house protocols. Comparison frames were generated by sliding of the aptamer sequence along the genome in increments of 1 nucleotide, resulting in genome fragments of the same length as the aptamers (typically 40 nt length each) that are to be compared with the aptamer sequences. In order not to miss any information at the 5′ and 3′ end, we also considered shorter frames obtained by overlaps of at least 12 nucleotide length of the 3′ end of the aptamer sequence with the 5′ end of the genomic sequence and vice versa. In particular, we start the alignment procedure by aligning the last nucleotide of the aptamer sequence with the first nucleotide at the 5′ end of the genome. The comparison frame in this case is a single nucleotide. Then the aptamer is slid one nucleotide at a time across the genome, increasing the comparison frame one nt at a time until its length is the same as that of the aptamer. This was done so as not to overlook potential stem-loop structures at the 5′ and 3′ end of the genomic sequence.

For each aptamer, we calculated the maximum Bernoulli score for its overlap with each of its comparison frames. The Bernoulli score B(L,N) is normailized so that it ranges from 0 to L, with L being the length of the aptamer. It can be converted to a probability via P(L,N)=(1/4)^{B(L,N)} which corresponds to the probability that a random sequence of B(L,N) letters would align precisely with the genome. The procedure identifies the largest fragment of the aptamer that has the highest Bernoulli score, and therefore, the lowest probability of having aligned to the genome fragment given by the comparison frame just by chance. The Bernoulli score (and associated probability) for a sequence of L letters to have N or fewer mismatches over the length of L nucleotides is calculated using the formula (Altschul & Erickson, 1986):

$$B ( L , N ) = L - {log}_{4} ( x )$$

$$x = {\sum }_{i = 0}^{N} ( \begin{matrix}L \\ i\end{matrix} ) {3}^{i}$$

Note that in most if not all cases, the fragment contributing to the score is smaller than the length of the aptamer, and contains some mismatches. For each comparison frame, the fragment of the aptamer which aligned to the genome with the maximum Bernoulli score was identified. If this maximum score was larger or equal to a threshold value corresponding to the most significant alignments, we logged it into the data file that was subsequently used to compute the histogram. The bioinformatics algorithm has been developed so that the threshold value can be adjusted depending on the needs of the user.

The histogram is then used to identify areas in the genome which are potential PSs. This is done by identifying the locations of the largest peaks in the histogram (or equivalently the genomic sequence) along with the aptamer which aligns to this area with the highest Bernoulli score. After having identified the set of aptamers which align to each peak with the highest Bernoulli score B(L,N), the corresponding areas of the genome are folded into stem-loops using Mfold (Zuker 2003). These are subsequently compared with the stem-loop structures of the most abundant aptamers obtained from next generation sequencing data. Finally, we also compute the statistical significance of the peaks (individual aptamer alignments) by comparing with the number of times that the consensus motif would occur in random sequences of the same length and letter content as the genomic sequence.

### EXAMPLE 1

We have shown using single molecule fluorescence spectroscopy assays of in vitro virus assembly that at nanomolar concentrations, e.g. approximating the conditions in vivo, there is packaging specificity with respect to the RNA for the model viruses bacteriophage MS2 and satellite tobacco necrosis virus (STNV). Assembly of capsids is also very precise and complete under these conditions. These observations mimic what is seen in vivo.

### EXAMPLE 2

The data from Example 1 can only be interpreted in terms of multiple interaction sites (PSs) between the cognate viral RNAs and their CPs that facilitate capsid assembly. We have worked out the molecular basis of such PS action for both MS2 and STNV [4-7].

### EXAMPLE 3

We have used RNA SELEX to identify putative PSs for a range of additional viruses, including TCV, BMV, and CCMV from plants, and HCV, HBV, HIV and HPeV from humans. In each case NextGen sequencing of the selected RNA pools yields millions of sequence reads that have been sorted and rank ordered by numbers of precise repeats of the same sequence. These individual sequences have been scanned against the cognate viral genome sequence as a reference. This yields multiple, statistically significant matches implying that there are multiple areas of each genome that have specific affinity for their cognate CPs.

### EXAMPLE 4

Mfold has been used to generate predicted secondary structures of the matching PSs within each genome. Moreover, aptamer Logos are generated using Clustl to identify consensus motifs. In every case so far the PSs fold into extended stem-loop regions in which the selected, previously random, regions play a significant role, often exhibiting sequence similarities/identities.

### EXAMPLE 5

For two viruses, Human Parecho virus (HPeV) and Turnip Crinkle virus (TCV), we have explored the affinity of the predicted PSs for their CPs and for the latter their effects on assembly. Specific binding (HPeV, Kd ˜100 nM) and in vitro capsid assembly (TCV) have been demonstrated for these viruses.

### EXAMPLE 6

Throughout the description the following terminology will be used:

aptamers for the RNA sequences identified via SELEX to bind to the coat protein target;
packaging signals (PS) for the regions in the viral genomes that the aptamers are aligning to with statistical significance.

Aptamer sequences will be represented by upper case letters and PSs by lower case letters. If a mix of upper and lower case letters occurs, this signifies that matches with the aptamer sequence have been superimposed on the genomic sequence to identify consensus motifs. Matches do not need to be contiguous in the RNA primary sequence.

As earlier work on bacteriophage MS2 demonstrates [7], the RNA sequences corresponding to PSs are only required to contain (not necessarily contiguous) motifs in order to be functional (e.g. an AxxA motif in the loop portion of a stem-loop, where x denotes any nucleotide).

### Human Parecho Virus (HPeV):

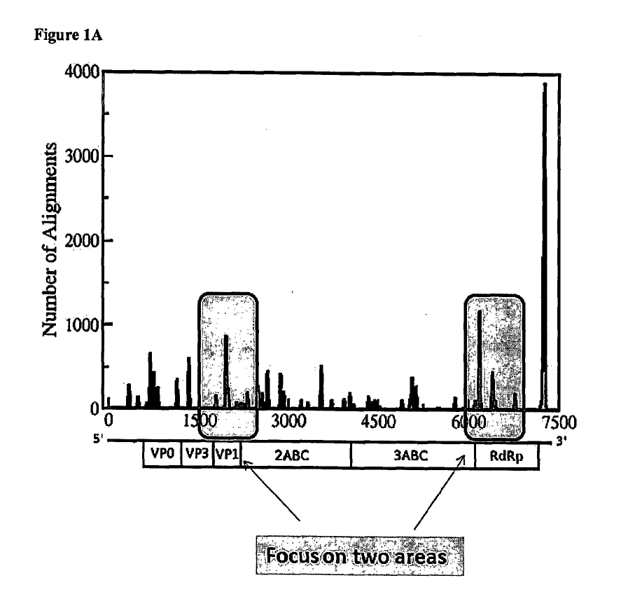

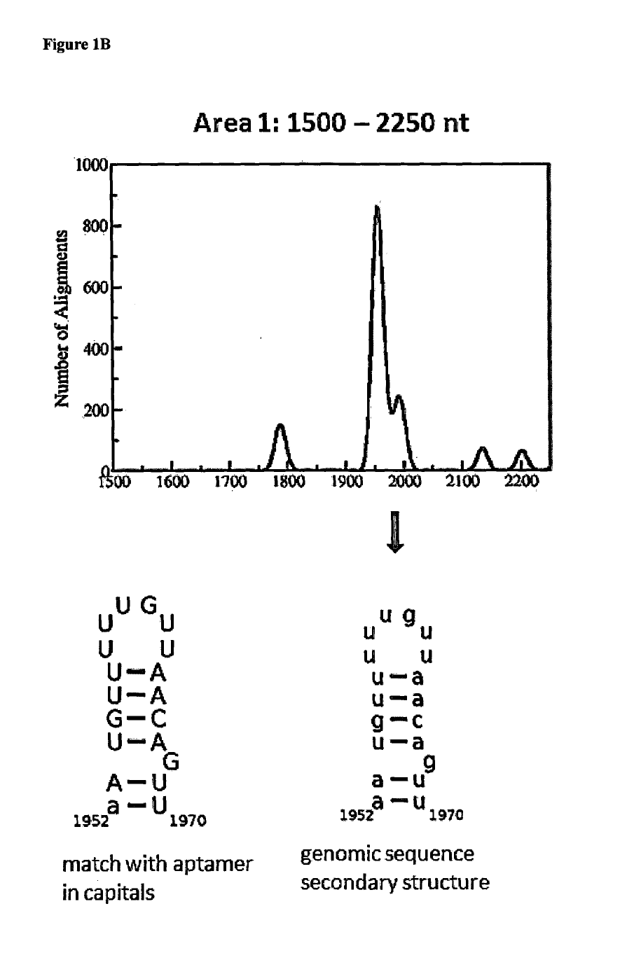

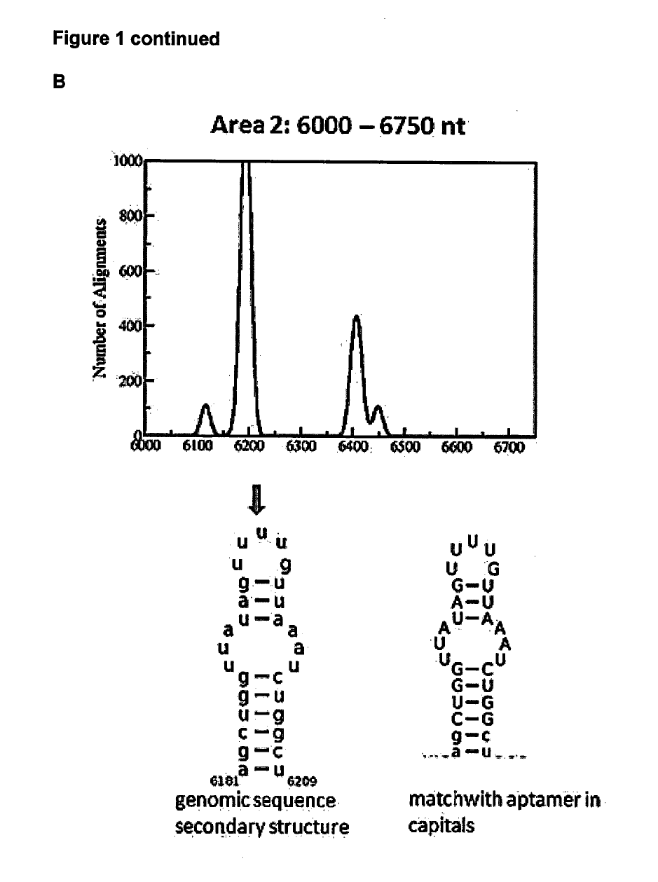

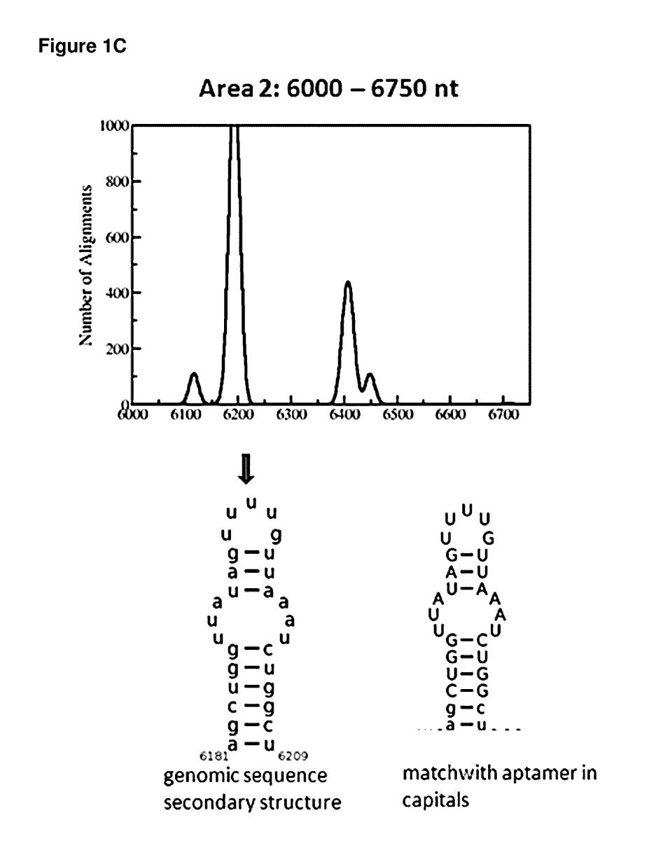

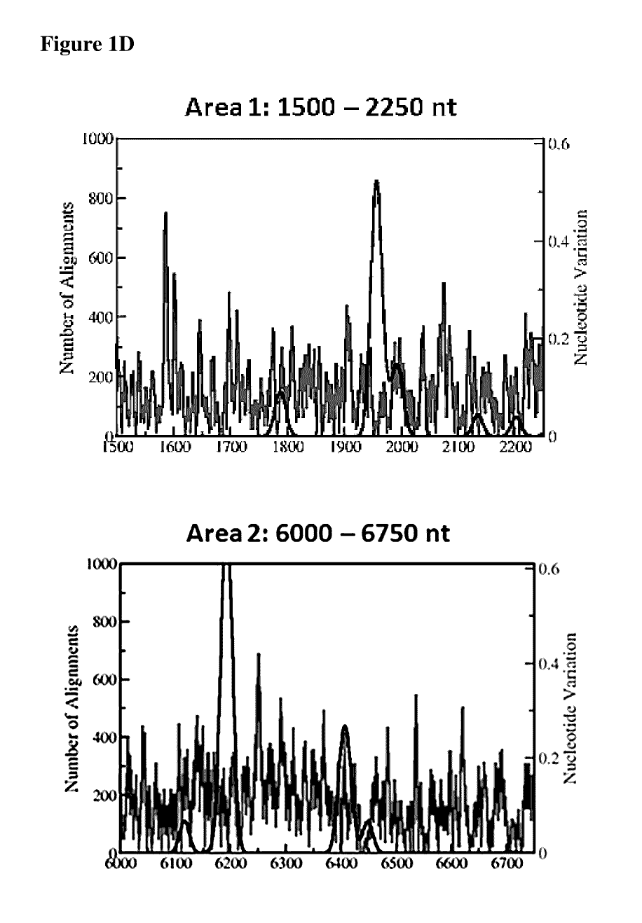

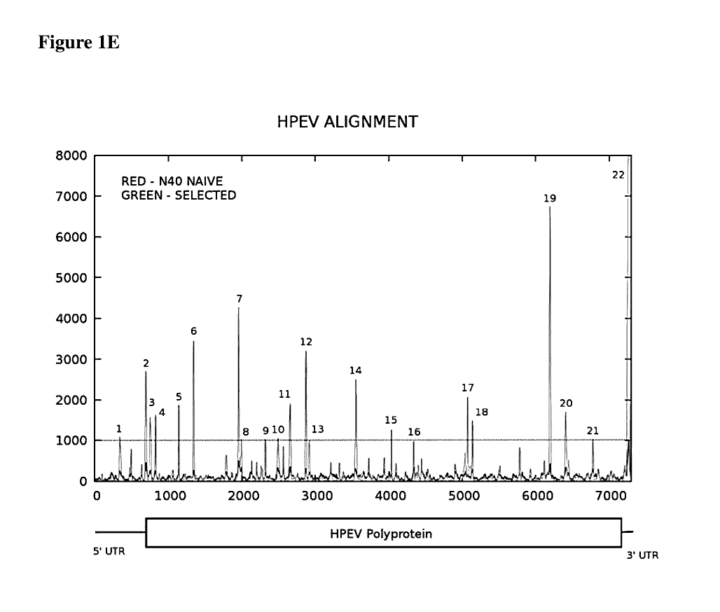

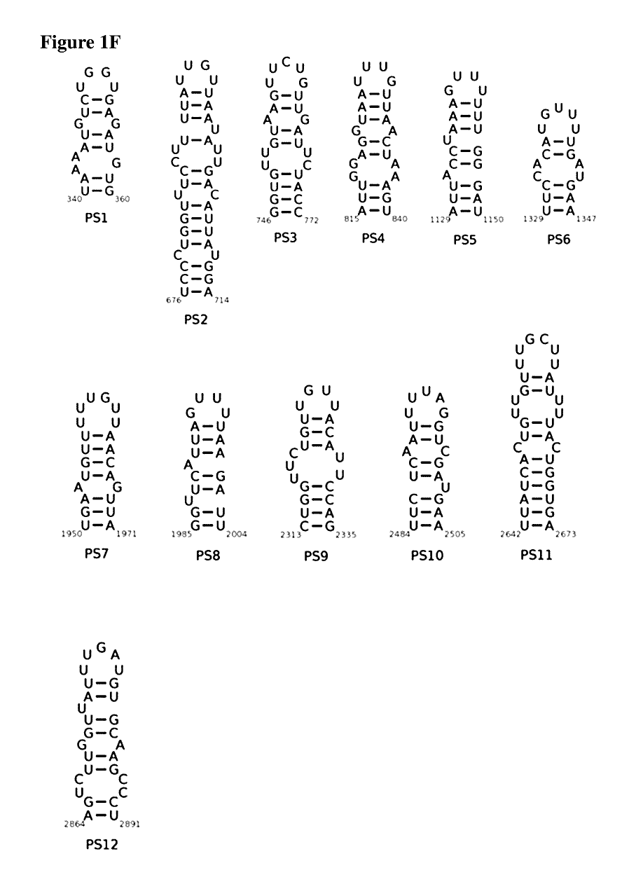

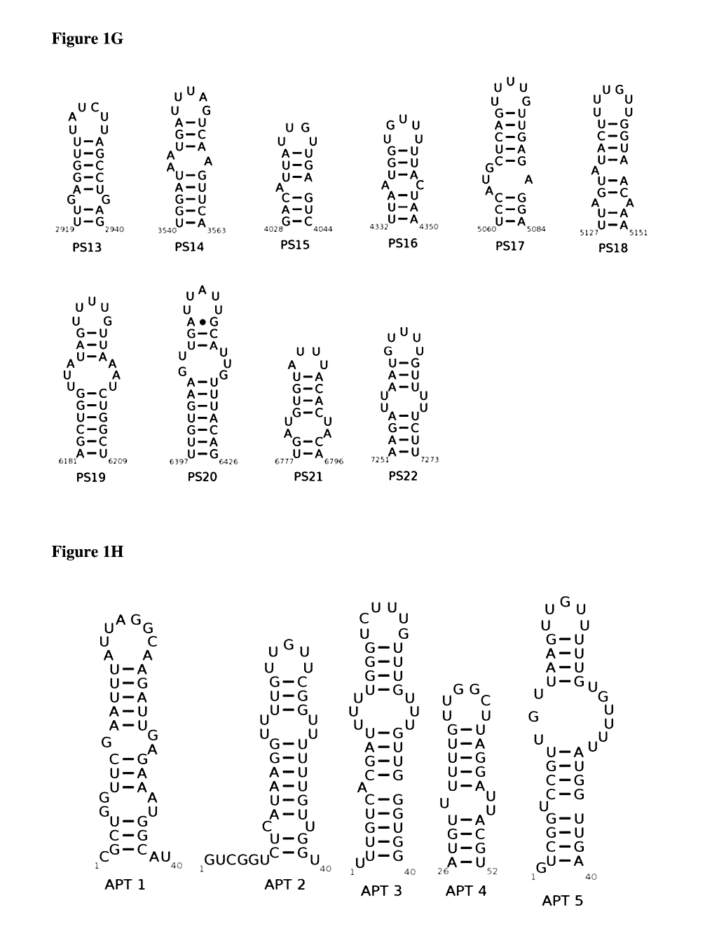

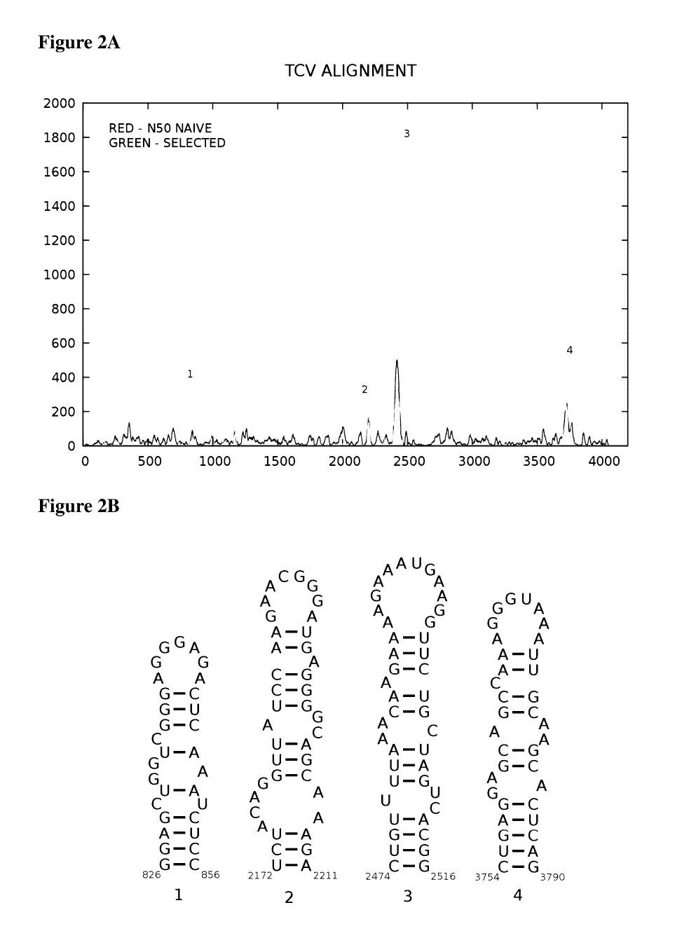

Aptamer alignment to the HPeV1 Harris genome (genebank id: L02971) resulted in the histogram plot in FIG. 1D. Only alignments with a Bernoulli scores of 12 or above are shown, because all others are not statistically significant (as random sequences also show hits of the same frequency with such scores). As demonstrated in FIG. 1H, we identified packaging signals as those peaks that have the largest possible Bernoulli scores (scores of 17 or 18 in this case). We checked that the areas thus identified correspond to conserved areas across all 21 available strains (FIG. 1A), as expected if these areas correspond to packaging signals with functional significance. We then folded these areas of the Harris genome via Mfold Among the different folds of similar energy returned by Mfold, we have chosen those that show the strongest similarity with the folds of the five most abundant aptamers returned by SELEX (FIG. 1H).

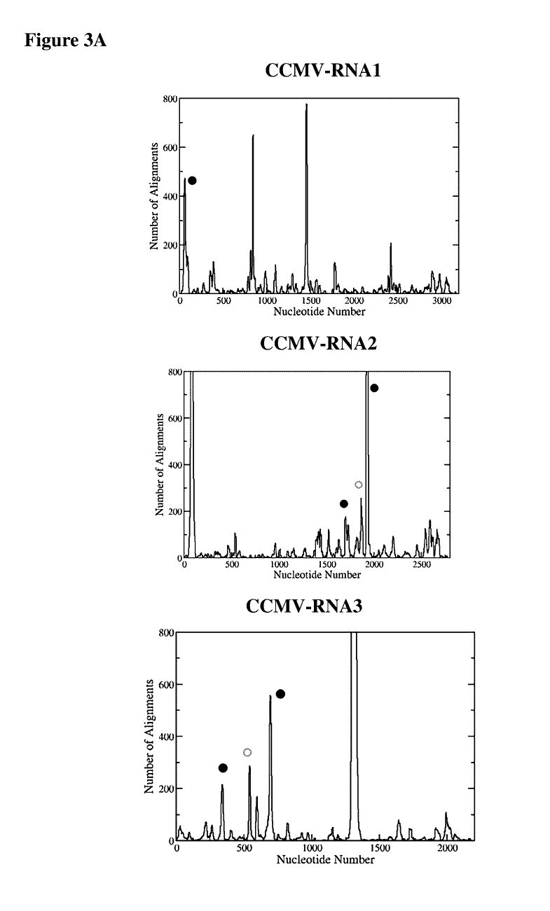

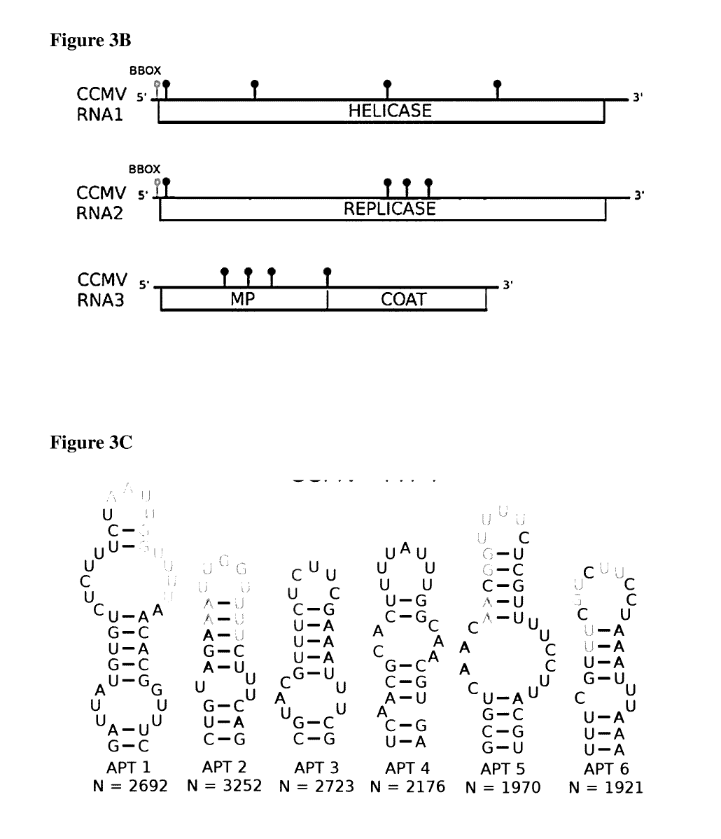

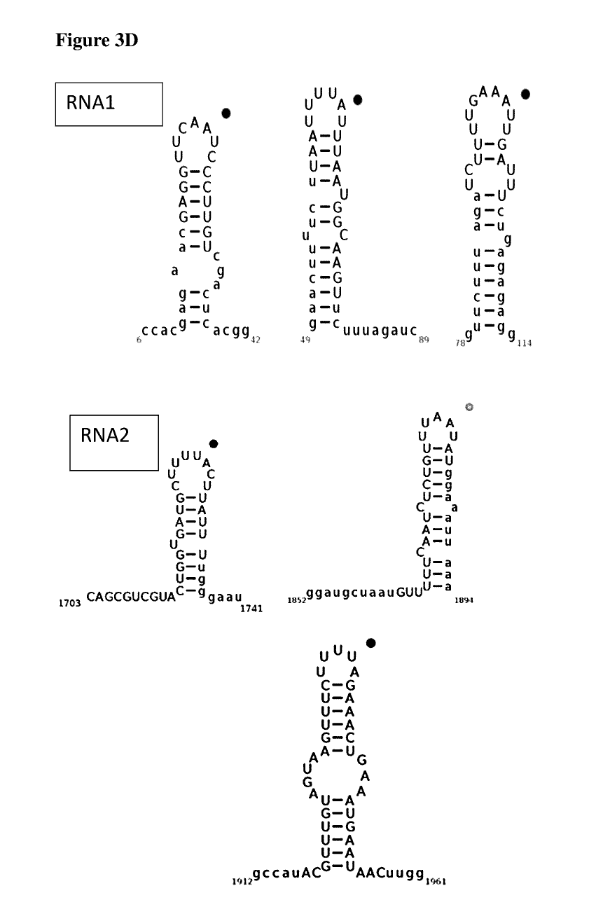

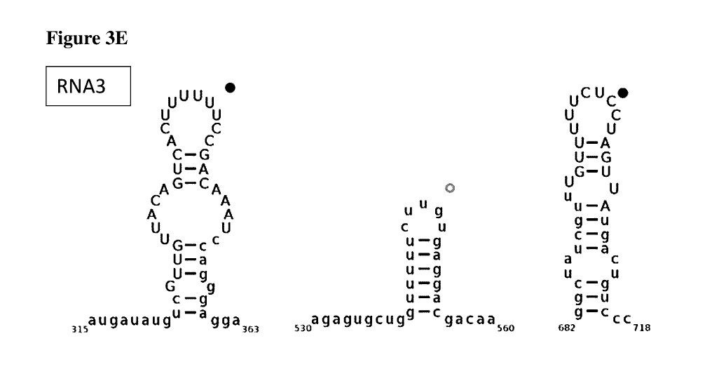

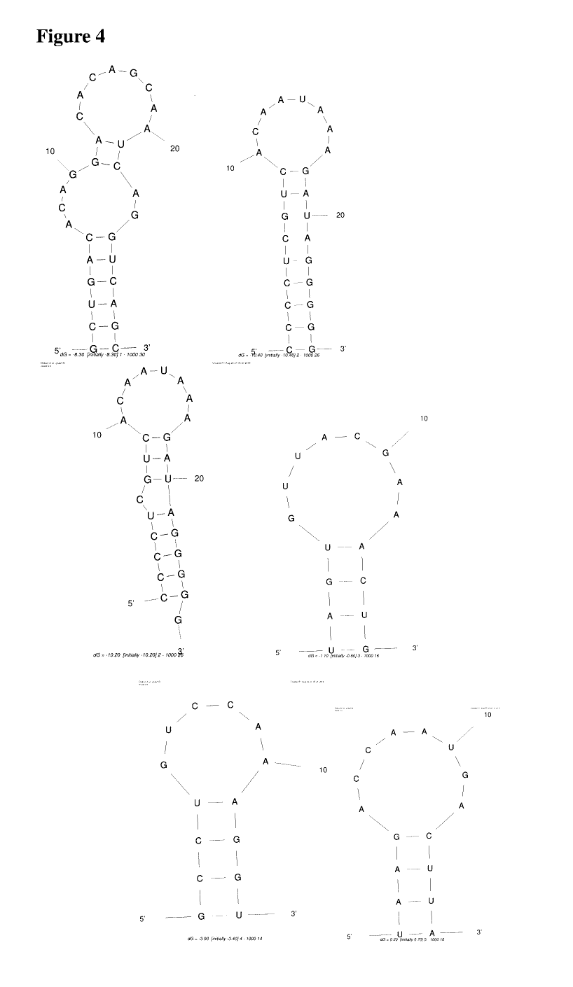

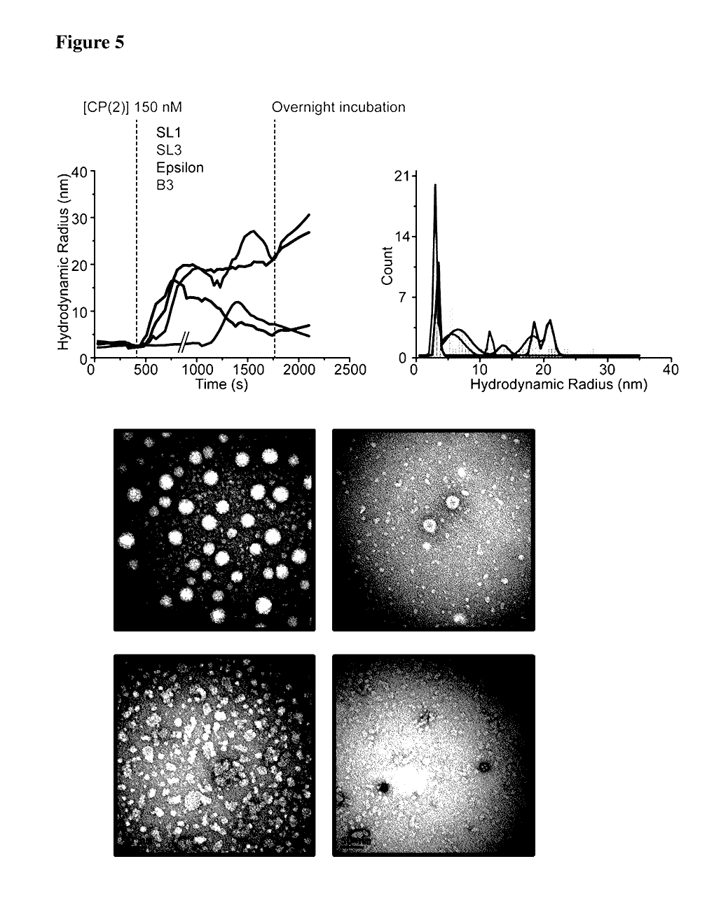

An alignment of the 9 stem-loops in FIG. 3D via Clustal identified characteristic poly-uridine motifs, e.g. UUUUGUU. The nucleotide composition of the genome was given by 29% U, 20% G, 18.8% C, and 1.9% A. The number of UUUG motifs expected in a genome with this composition was (on average) 36. The number of UUUG motifs in the Harris genome is 44, pointing to the fact that this motif could be significant. This is then probed via experiment (binding and assembly assays).

We performed the following statistical test: Each peak area in the black curve coincides with minima of value 0 in the red curve, i.e. an area of at least 5 perfectly aligned nucleotides across the 21 genomes. The chance of having perfect alignment (i.e. a value of 0 in the red curve) is 429/7339, i.e. 0.058%; the chance that any given nucleotide is part of a peak area is approximately 1036/7339, i.e. 14%, and significantly reduced if required to be central to the peak area. Hence, the overall chance of having an area with perfect alignment (zero value of red curve) in a peak area in the black curve is 0.8%, and the chance of finding this 26 times in the genome is 0.8²⁶%, i.e. very small. This implies that these alignments are significant.

We have established that one of these PSs binds its capsid protein specifically with an affinity in the nanomolar range.

### Human Immunodeficiency Virus (HIV):

HIV assembly takes place in two stages. First, GAG protein assembles a protein shell around the bipartite RNA genome. Then GAG cleaves into three domains: the nucleocapsid domain (NC domain) that is in complex with the genomic RNA; the middle domain (CA domain); and the out (MA) domain. At this stage, CA assembles the distinctive cone structure characteristic of mature HIV particles around the RNA-NC complex and inside the spherical shell defined by the MA domain. The assembly of HIV capsid is reviewed in Bell N M & Lever A M C, (2013), Trends in Microbiology Volume (21) (3).

It has been shown previously that there exists a packaging signal in the region towards the 5′ end (Psi) that binds the NC domain of GAG. The structural determinants of the high affinity binding site within the HIV-Psi element have been characterised with different experimental techniques (Berglund et al, 1997; Clever et al., 2000; Fisher et al., 1998). Based on these studis, a characteristic G-x-G motif, where x can be any nucleotide, has been suggested to account for affinity of Psi to NC and is present in all four stem-loops of the Psi packaging site. Further analysis (Lodwell et al., 2000; Paoletti et al., 2002; Yuan et al., 2003; Webb et al., 2013) suggests that the motif does not need to be connected, but that variants including G-x in a single-stranded bulge, followed by G in the loop of a stem-loop, and locations of the G-x-G in both loops and bulges are possible.

Given this information, we did not perform a SELEX analysis for this virus as for the others, but rather searched for the G-x-G motifs (in all its allowed variants) in the published secondary structure of the entire HIV-1 RNA genome (Watts et al, 2009) in order to identify all packaging signals that bind to the NC domain of GAG during stage 1. We performed a bioinformatics analysis similar to the one outlined above to establish that this motif occurs with statistical significance across the genome, and we identified the locations of the putative multiple degenerate packaging signals with that motif across the genome. We hypothesize that they are playing an active role as packaging signals during stage 1 of the assembly process (hence termed by us primary packaging signals). This idea of multiple degenerate packaging signals in HIV is new, as it is also for all the other viruses exemplified here.

We used these results to identify which areas of the genome are likely to be in complex with the NC domain at the onset of stage 2 of the assembly process. We then analysed the remaining regions (i.e. those not in complex with NC) for possible binding sites to the CA domain that could play the role of packaging signals during cone formation. For this we isolated all stem-loops (39, see table) in the secondary structure not in complex with NC at the onset of stage 2 and preformed a similarity analysis (see weblogo) which shows a clear bias towards a specific common motif (A-rich loop). Since CA binding can only occur during stage 2 after GAG cleavage, these are termed by us secondary packaging signals.

i) Plant Viruses:

Turnip Crinkle virus (TCV):

The analysis of the TCV genome has been performed following the same protocol as above. In this case, the histogram plot shows a number of packaging signals located in close proximity of each other that we label as Pair 1-Pair 3; in addition, there are 5 packaging signals that we term S1-S5. Our discovery of multiple packaging signals and their pairing sheds new light on the assembly mechanism. The distinctive pattern of packaging signal pairs suggests that pairs may have a specific functional role, perhaps in bracketing protein dimers and hence aiding with capsid assembly.

### Cowpea Chlorotic Mottle Virus (CCMV):

The analysis of the three CCMV genomes (CCMV1-CCMV3) has been performed following the same protocol as above. The histogram plot shows a number of peaks above the cut-off marking statistical-significant hits. The analysis of the peaks is still in progress, which is why we are indicating sequences containing packaging signals rather than the packaging signals themselves at this stage. However, for all peaks already analysed stem-loops with a clear consensus motif are visible. An analysis of SELEX data derived at different pH values shows their occurrence at pH4.5, but not at pH7, as expected from reassembly assays which show different assembly behaviours at these pH values. Our analysis is hence consistent with their expected function as packaging signals.

### Human Parecho Virus (HPeV):

TABLE 1
Sequences of HPeV PSs (based on viral
strain Human Parechovirus 1 (aka Human
Echovirus 22 or Harris strain).

| SEQ | | | |
|---|---|---|---|
| ID | Start | End | Sequence |
| 1 | 666 | 690 | 5′ AGGGGGGAUCCCUGGUUUCCUUU 3′ |
| 2 | 1329 | 1347 | 5′ UUCCACAUGUUUUGAUGAA 3′ |
| 3 | 1950 | 1971 | 5′ UGAAUGUUUUUGUUAACAGUUA 3′ |
| 4 | 2484 | 2505 | 5′ UUCUCAAUUUUAGGUCGAUGAA 3′ |
| 5 | 4332 | 4350 | 5′ UUAAUGGUGUUUUUACUAA 3′ |
| 6 | 5127 | 5151 | 5′ UUAGUAUACUUUUGUUGGUAACAAA 3′ |
| 7 | 6181 | 6209 | 5′ AGCUGGUUAUAGUUUUGUUAAAUCUGGCU 3′ |
| 8 | 6403 | 6432 | 5′ AGGCUUGUGAAGUUGAUUAUUGCAUUGUUU 3′ |
| 9 | 7251 | 7273 | 5′ AAGAUUAAUGUUUUGUUUUUCUU 3′ |

TABLE 2
Sequences of HPeV aptamers
identified via SELEX.

| SEQ ID | Aptamer No | Sequence |
|---|---|---|
| 10 | 1 | 5′ CGCUGGUUCGAAUUUAUUAGGCAA |
| | | GAUUGAGAAAUGGCU 3′ |
| 11 | 2 | 5′ GUCGGUCUCAUAAGGUUUUGUUGU |
| | | UCGGUUUUUUGUUGGU 3′ |
| 12 | 3 | 5′ UUCUCACGAUUUUUGGGUCUUUGU |
| | | UUGUUUGUUGGGUGG 3′ |
| 13 | 4 | 5′ AUGUUUUUUGUUGGCUUAGGAUUA |
| | | CGU 3′ |
| 14 | 5 | 5′ GUCGGUCCGUUGUUAAGUUGUUUU |
| | | UGUGUUUUAUGGUUGA 3′ |

### Human Immunodeficiency Virus (HIV):

TABLE 3 (a)
Sequences of HIV PSs (based on viral strain NL4-3).

| SEQ | | | |
|---|---|---|---|
| ID No | Start | End | Sequence |
| 15 | 138 | 178 | AGATCCCTCAGACCCTTTTAGTCAGTGTGGAAAATCTCTAG |
| 16 | 1076 | 1100 | AGGATGGATGACACATAATCCACCT |
| 17 | 1214 | 1247 | TAGAGACTATGTAGACCGATTCTATAAAACTCTA |
| 18 | 1645 | 1672 | TCTGGCCTTCCCACAAGGGAAGGCCAGG |
| 19 | 1729 | 1757 | TTGGGGAAGAGACAACAACTCCCTCTCAG |
| 20 | 1823 | 1849 | CCCCTCGTCACAATAAAGATAGGGGGG |
| 21 | 2145 | 2171 | ATGGCCCAAAAGTTAAACAATGGCCAT |
| 22 | 2245 | 2260 | TGGGCCTGAAAATCCA |
| 23 | 2268 | 2310 | CTCCAGTATTTGCCATAAAGAAAAAAGACAGTACTAAATGGAG |
| 24 | 2328 | 2348 | GAGAACTTAATAAGAGAACTC |
| 25 | 2781 | 2802 | GGATGGGTTATGAACTCCATCC |
| 26 | 2811 | 2835 | GGACAGTACAGCCTATAGTGCTGCC |
| 27 | 2629 | 2654 | AGTCATCTATCAATACATGGATGATT |
| 28 | 3336 | 3358 | GGGAGTTTGTCAATACCCCTCCC |
| 29 | 3840 | 3890 | TGGCTAGTGATTTTAACCTACCACCTGTAGTAGCAAAAGAAATAGTAGC |
| | | | CA |
| 30 | 4072 | 4095 | CTTCCTCTTAAAATTAGCAGGAAG |
| 31 | 4642 | 4674 | ACACATGGAAAAGATTAGTAAAACACCATATGT |
| 32 | 4694 | 4732 | GGACTGGTTTTATAGACATCACTATGAAAGTACTAATCC |
| 33 | 5234 | 5265 | CAACATATCTATGAAACTTACGGGGATACTTG |
| 34 | 5303 | 5343 | CTGCTGTTTATCCATTTCAGAATTGGGTGTCGACATAGCAG |
| 35 | 5499 | 5519 | GCCTTAGGCATCTCCTATGGC |
| 36 | 5530 | 5581 | GGAGACAGCGACGAAGAGCTCATCAGAACAGTCAGACTCATCAAGCT |
| | | | TCTCT |
| 37 | 6270 | 6290 | GGTGCAGAAAGAATATGCATT |
| 38 | 6711 | 6757 | TGTTACAATAGGAAAAATAGGAAATATGAGACAAGCACATTGTAACA |
| 39 | 6475 | 6497 | TGTACAAATGTCAGCACAGTACA |
| 40 | 6536 | 6598 | TGCTGTTAAATGGCAGTCTAGCAGAAGAAGATGTAGTAATTAGATCTGC |
| | | | CAATTTCACAGACA |
| 41 | 6870 | 6886 | AATTGTAACGCACAGTT |
| 42 | 6983 | 7016 | CTGAAGGAAGTGACACAATCACACTCCCATGCAG |
| 43 | 7079 | 7099 | GTGGACAAATTAGATGTTCAT |
| 44 | 7438 | 7464 | GAGGCGCAACAGCATCTGTTGCAACTC |
| 45 | 7468 | 7493 | GTCTGGGGCATCAAACAGCTCCAGGC |
| 46 | 8053 | 8077 | CTCTTCAGCTACCACCGCTTGAGAG |
| 47 | 8455 | 8505 | AGCAATCACAAGTAGCAATACAGCAGCTAACAATGCTGCTTGTGCCTG |
| | | | GCT |
| 48 | 8551 | 8565 | GGTACCTTTAAGACC |
| 49 | 8578 | 8597 | GGCAGCTGTAGATCTTAGCC |
| 50 | 8723 | 8751 | CCAGGGGTCAGATATCCACTGACCTTTGG |
| 51 | 8753 | 8773 | TGGTGCTACAAGCTAGTACCA |
| 52 | 9042 | 9057 | GCTGCATATAAGCAGC |
| 53 | 9141 | 9170 | AAGCCTCAATAAAGCTTGCCTTGAGTGCTT |

TABLE 3 (b)
Sequences of HIV PSs
(based on viral strain HxB2).

| SEQ | | | |
|---|---|---|---|
| ID | | | |
| NO: | | | Sequence |
| 573 | 693 | 723 | 5′ GCUGACACAGGACACAGCAAUCAGGUCAGC 3′ |
| 574 | 1823 | 1849 | 5′ CCCCUCGUCACAAUAAAGAUAGGGGG 3′ |
| 575 | 5078 | 5094 | 5′ UAGUGUUACGAAACUG 3′ |
| 576 | 6380 | 6394 | 5′ GCCUGUCCAAAGGU 3′ |
| 577 | 8569 | 8585 | 5′ UAAGACCAAUGACUUA 3′ |

### Turnip Crinkle Virus (TCV):

TABLE 4
Sequences of TCV PSs.

| | PS | | | |
|---|---|---|---|---|
| SEQ ID NO. | Name | Start | End | Sequence |
| 54 | P1a | 244 | 283 | 5′ GGGACGUAUAGUAAUAGAGGUCAGAUAGGUAGUAGUCUC 3′ |
| 55 | P1b | 337 | 372 | 5′ UAGGUUGGUAGGAACGGAAGAGGAAGCCACAUCCUG 3′ |
| 56 | S1 | 819 | 859 | 5′ CUUGCGGGAGCUGGUCGGGAGGGAGACUCAAAUCUCCAGG 3′ |
| 57 | S2 | 973 | 1008 | 5′ ACUCAACAAUUUGAGAAGAGGGUUGAUGGAAAGAGU 3′ |
| 58 | S3 | 1150 | 1176 | 5′ GUCGUUCUACAAGGGCAGGAGGGCCAC 3′ |
| 59 | S4a | 2128 | 2158 | 5′ GGACUACAAGAAGAAGAUGCAAGAUGUUUCC 3′ |
| 60 | S4b | 2192 | 2219 | 5′ GGGAUGAGGGGCAGCAAAGACGUGUCCC 3′ |
| 61 | P2a | 2398 | 2441 | 5′ GACGCAACAGGAAAACGGAAGAAAGGCGGAGAGAAAAGUGCGAA 3′ |
| 62 | P2b | 2471 | 2518 | 5′ |
| | | | | GCUCUGUUUUAAACAAGAAAAGAAAUGAAGGUUCUGCUAGUCACGGG |
| | | | | G3′ |
| 63 | S5a | 3487 | 3531 | 5′ |
| | | | | AGAUUGGGCAGUUCGCAGGUGUUAAGGACGGACCCAGGCUGGUUU |
| | | | | 3′ |
| 64 | S5b | 3531 | 3571 | 5′ UCAUGGUCCAAGACCAAGGGGACAGCUGGGUGGGAGCACGA 3′ |
| 65 | P3a | 3694 | 3733 | 5′ GUGUCCAAUGGGCAGGAGUGAAGGUAGCAGAAAGGGGACA 3′ |
| 66 | P3b | 3754 | 3790 | 5′ CUGAGGAGCAGCCAAAGGGUAAAUUGCAAGCACUCAG 3′ |
| 472 | | | | ggagcugguc gggagggaga cucaaaucuc c |
| 473 | | | | ucuacagguu auccaagaac gggaugaggg gcugcaaaga |
| 474 | | | | cuguuuuaaa caagaaaaga aaugaagguu cugcuaguca cgg |
| 475 | | | | cugaggagca gccaaagggu aaauugcaag cacucag |

TABLE 5
TCV aptamers identified via SELEX.

| SEQ ID NO: | Aptamer N | Sequence |
|---|---|---|
| 67 | 1 | 5′ GGCAAACGGUAAGGCCAAAAGGGAC |
| | | GAGGGUAGAGAUUGAUAGAAAGCC 3′ |
| 68 | 2 | 5′ GCAACUAGGAAAAGGGAAGG |
| | | GCAAGGGAAGGGACCGAAGAGCAGC 3′ |
| 69 | 3 | 5′ GGCAACUAACAAGAGGGAGG |
| | | AGAGGGAGGAACGUUAGGGUAGCC 3′ |

### Cowpea Chlorotic Mottle Virus (CCMV):

TABLE 6
Sequences containing packaging signals of CCMV1 PSs.

| SEQ ID NO: | | |
|---|---|---|
| 70 | 63 | 5′ |
| | | GTAATCCACGAGAACGAGGTTCAATCCCTTGTCGACTCACGGAGTATCGAACTTTT |
| | | CTTAATTTTATTTAATGGCAAGTTCTTTAGATCTTTTGAAATTGATTTCTGAGAGAGG |
| | | CGCTGACAGCCGAGGCGCTTCGGACATAGTTGAACAACAAGCTGTAAAG 3′ |
| 71 | 361 | 5′ |
| | | ATGGAGGAGCTTTTGATTTGAACTTAACTCAACAATATAATGCTCCCCATAGTTTGG |
| | | CTGGAGCTCTGCGAATAGCGGAGCATTATGACTGTCTTTCAAGCTTCCCCCCTCTT |
| | | GATCCCATCATTGATTTTGGTGGTTCTTGGTGGCATCATTATTCCAGGAAGGACAC |
| | | ACGTATTCACAGTTGTTGTCCCGTGTTGGGCG 3′ |
| 72 | 409 | 5′ |
| | | ATAGTTTGGCTGGAGCTCTGCGAATAGCGGAGCATTATGACTGTCTTTCAAGCTTC |
| | | CCCCCTCTTGATCCCATCATTGATTTTGGTGGTTCTTGGTGGCATCATTATTCCAGG |
| | | AAGGACACACGTATTCACAGTTGTTGTCCCGTGTTGGGCGTCAGAGATGCTGCTC |
| | | GACATGAAGAACGACTATGTAGAATGCGTAAGT 3′ |
| 73 | 815 | 5′ |
| | | CGAAGGCGTTTTACCTTTGTTGAAGTGCCGTTGGATGAAGTCTGGGAAAGGTAAA |
| | | TCTGAGGTCATTAAATTTGATTTCATGAATGAGAGCACACTTTCTTATATTCATTCTT |
| | | GGACCAATCTTGGTTCATTTTTGACTGAGTCTGTGCATGTGATAGGAGGTACTACTT |
| | | ATCTCCTAGAACGTGAGCTCTTAAAATGCAA 3′ |
| 74 | 845 | 5′ |
| | | TTGGATGAAGTCTGGGAAAGGTAAATCTGAGGTCATTAAATTTGATTTCATGAATGA |
| | | GAGCACACTTTCTTATATTCATTCTTGGACCAATCTTGGTTCATTTTTGACTGAGTCT |
| | | GTGCATGTGATAGGAGGTACTACTTATCTCCTAGAACGTGAGCTCTTAAAATGCAAT |
| | | ATTATGACCTATAAAATCGTTGCCACAAA 3′ |
| 75 | 994 | 5′ |
| | | AACGTGAGCTCTTAAAATGCAATATTATGACCTATAAAATCGTTGCCACAAATCTGAA |
| | | GTGTCCTAAGGAAACGTTGCGACATTGTGTTTGGTTTGAGAATATTTCCCAATATGT |
| | | CGCCGTTAACATTCCTGAAGACTGGAATCTGACTCATTGGAAACCCGTACGTGTG |
| | | GCAAAAACCACCGTAAGAGAGGTTGAAGAGA 3′ |
| 76 | 1102 | 5′ |
| | | AATATGTCGCCGTTAACATTCCTGAAGACTGGAATCTGACTCATTGGAAACCCGTA |
| | | CGTGTGGCAAAAACCACCGTAAGAGAGGTTGAAGAGATTGCTTTTCGATGTTTTAA |
| | | GGAGAATAAAGAGTGGACGGAGAATATGAAAGCGATAGCATCTATTCTGTCCGCTA |
| | | AATCTTCTACAGTCATTATCAACGGTCAAGCTA 3′ |
| 77 | 1299 | 5′ |
| | | GCTATCATGGCCGGAGAGAGGCTGAACATTGATGAGTATCATCTCGTCGCCTTTGC |
| | | TCTCACTATGAATTTGTATCAGAAATATGAAAATATTCGGAATTTTTATAGTGAGATGG |
| | | AATGGAAGGGCTGGGTCAACCACTTTAAAACTAGATTTTGGTGGGGAGGAAGTAC |
| | | GGCTACCTCAAGCACTGGTAAGATTCGAGAG 3′ |
| 78 | 1466 | 5′ |
| | | TACGGCTACCTCAAGCACTGGTAAGATTCGAGAGTTTCTGGCTGGTAAATTCCCTT |
| | | GGCTGAGGTTAGATTCGTACAAAGACAGTTTTGTTTTTCTGTCGAAGATCTCTGAT |
| | | GTCAAAGAGTTTGAGAACGATTCTGTTCCCATCTCCAGACTGAGGAGTTTCTTCAG |
| | | CAGTGAGGACCTCATGGAGCGCATTGAATTAGA 3′ |
| 79 | 1794 | 5′ |
| | | AAGGAGCCTAAACCGGAAGTGACCGTTGGAGCTGAACCAACAGGCCCCGAAGAG |
| | | GCATCGAGACACTTTGCCATCAAGGAATTCTCTGATTATTGTCGTCGCCTTGACTG |
| | | TAACGCTGTGTCAAATCTTCGTCGTTTATGGGCCATTGCTGGCTGCGATGGGAGG |
| | | ACTGCGAGAAATAAGTCGATCCTTGAAACTTATCAT 3′ |
| 80 | 2439 | 5′ |
| | | CTACACTATGGTCAGCTGCTCGCTGTGGCTGCTCTCTGTAAGTGTCAGTCTGTTCT |
| | | TGCATTCGGAGACACGGAGCAAATTTCTTTTAAATCGCGAGATGCAACTTTCCGCC |
| | | TGAAATATGGTGATTTGCAGTTTGACAGTCGCGATATTGTTACGGAGACATGGAGA |
| | | TGTCCGCAAGATGTTATTTCCGCAGTTCAGACT 3′ |
| 81 | 2905 | 5′ |
| | | TGGTAAGACTTAAATCTACCAAGTGTGATCTATTTAAAACTGAAGAATATTGCTTGGT |
| | | GGCTTTGACTCGACATAAGATTACCTTTGAGTATCTTTATGTTGGTATGCTATCAGG |
| | | TGATTTAATATTTAGAAGTATATCTTGATCCTGAGTGTGATTCACTTACGAATCAGTT |
| | | CTAACGGTTTCTATAAACCGTAGTCGTC 3′ |
| 82 | 2988 | 5′ |
| | | TTTGAGTATCTTTATGTTGGTATGCTATCAGGTGATTTAATATTTAGAAGTATATCTTG |
| | | ATCCTGAGTGTGATTCACTTACGAATCAGTTCTAACGGTTTCTATAAACCGTAGTCG |
| | | TCGTTGCGACGCCGACCGTCTTACAAGACGTTCGAGCTGCCTTTGGGTTTTACTC |
| | | CTTGAACCCTTCAGAAGAATTCTTCGGAGT 3′ |

TABLE 7
Sequences containing packaging signals of CCMV2 PSs.

| SEQ | | |
|---|---|---|
| ID | | |
| NO: | | |
| 83 | 78 | 5′ |
| | | GTAATCCACGAGAGCGAGGTTCAATCCCTTGTCGACTCACGGGTCTCCATCAGTT |
| | | GAAAACAGTTTATACATTTTCTTCTTGATATTTTTCTTCTTTACTTCCATTAATATGTCT |
| | | AAGTTCATTCCAGAAGGTGAGACTTACCACGTTCCCTCATTCCAATGGATGTTTGA |
| | | TCAGACT 3′ |
| 84 | 555 | 5′ |
| | | GACGGTTCATTCGTTGATGAATCTGAGTGTGACGATTGGCGGCCGGTAGATACCT |
| | | CTGATGGTTTCACCGAAGCAATGTTTGATGTGATGAATGAGATTCCTGGCGAGGA |
| | | AACAAAAAATACATGCGCTTTAAGTCTTGAAGCTGAATCAAGGCAAGCTCCAGAAA |
| | | CTTCCGATATGGTGCCGTCTGAATATACGTTGGCA 3′ |
| 85 | 1404 | 5′ |
| | | AAGTCTGATATTAAACCAGTTGTCTCGGATACGTTACACCTCGAACGAGCTGTTGC |
| | | TGCAACAATAACATTTCATGGTAAAGGAGTTACTAGCTGCTTCTCACCATATTTTAC |
| | | GGCTTGTTTCGAGAAGTTTTCAAAAGCTTTAAAATCAAGGTTTGTGGTCCCCATAG |
| | | GGAAGATCTCCTCCCTGGAACTGAAAAATGTT 3′ |
| 86 | 1447 | 5′ |
| | | AACGAGCTGTTGCTGCAACAATAACATTTCATGGTAAAGGAGTTACTAGCTGCTTC |
| | | TCACCATATTTTACGGCTTGTTTCGAGAAGTTTTCAAAAGCTTTAAAATCAAGGTTT |
| | | GTGGTCCCCATAGGGAAGATCTCCTCCCTGGAACTGAAAAATGTTCCCCTCTCGA |
| | | ATAAATGGTTTCTTGAGGCGGATTTGAGTAAGT 3′ |
| 87 | 1534 | 5′ |
| | | TTTCAAAAGCTTTAAAATCAAGGTTTGTGGTCCCCATAGGGAAGATCTCCTCCCTG |
| | | GAACTGAAAAATGTTCCCCTCTCGAATAAATGGTTTCTTGAGGCGGATTTGAGTAA |
| | | GTTTGATAAATCTCAGGGTGAGCTTCATCTTGAGTTCCAAAGAGAGATATTGTTGT |
| | | CATTGGGTTTTCCAGCCCCTTTGACTAATTGGT 3′ |
| 88 | 1637 | 5′ |
| | | TTTGAGTAAGTTTGATAAATCTCAGGGTGAGCTTCATCTTGAGTTCCAAAGAGAGA |
| | | TATTGTTGTCATTGGGTTTTCCAGCCCCTTTGACTAATTGGTGGTGTGATTTCCATA |
| | | GGGAATCTATGCTATCGGATCCTCATGCTGGAGTTAACATGCCAGTTTCCTTTCAG |
| | | CGTCGTACTGGTGATGCTTTTACTTATTTTGG3′ |
| 89 | 1702 | 5′ |
| | | CATTGGGTTTTCCAGCCCCTTTGACTAATTGGTGGTGTGATTTCCATAGGGAATCT |
| | | ATGCTATCGGATCCTCATGCTGGAGTTAACATGCCAGTTTCCTTTCAGCGTCGTAC |
| | | TGGTGATGCTTTTACTTATTTTGGGAATACTTTGGTGACTATGGCCATGATGGCCTA |
| | | TTGTTGCGATATGAACACCGTGGACTGTGCTA 3′ |
| 90 | 1745 | 5′ |
| | | CCATAGGGAATCTATGCTATCGGATCCTCATGCTGGAGTTAACATGCCAGTTTCCT |
| | | TTCAGCGTCGTACTGGTGATGCTTTTACTTATTTTGGGAATACTTTGGTGACTATGG |
| | | CCATGATGGCCTATTGTTGCGATATGAACACCGTGGACTGTGCTATCTTTTCCGGT |
| | | GATGATTCTCTGTTAATTTGTAAAAGTAAACC 3′ |
| 91 | 1837 | 5′ |
| | | GGAATACTTTGGTGACTATGGCCATGATGGCCTATTGTTGCGATATGAACACCGTG |
| | | GACTGTGCTATCTTTTCCGGTGATGATTCTCTGTTAATTTGTAAAAGTAAACCACAT |
| | | CTGGATGCTAATGTTTTTCAATCTCTGTTTAATATGGAAATTAAAGTTATGGACCCAA |
| | | GTTTGCCATACGTTTGTAGTAAGTTTCTTT 3′ |
| 92 | 1869 | 5′ |
| | | TATTGTTGCGATATGAACACCGTGGACTGTGCTATCTTTTCCGGTGATGATTCTCT |
| | | GTTAATTTGTAAAAGTAAACCACATCTGGATGCTAATGTTTTTCAATCTCTGTTTAAT |
| | | ATGGAAATTAAAGTTATGGACCCAAGTTTGCCATACGTTTGTAGTAAGTTTCTTTTA |
| | | GAAACTGAAATGAATAACTTGGTGTCTGTG 3′ |
| 93 | 1945 | 5′ |
| | | CACATCTGGATGCTAATGTTTTTCAATCTCTGTTTAATATGGAAATTAAAGTTATGGA |
| | | CCCAAGTTTGCCATACGTTTGTAGTAAGTTTCTTTTAGAAACTGAAATGAATAACTT |
| | | GGTGTCTGTGCCTGATCCTATGAGAGAGATACAGAGACTGGCTAAGCGAAAGATC |
| | | ATCAAATCGCCTGAGTTGTTAAGAGCCCACT 3′ |
| 94 | 2205 | 5′ |
| | | TTATTATGCAAGTTTGTGGCTCTCAAGTATAAAAAACCTGACGTTGAAAACGATGTC |
| | | AGAGTAGCCATTGCTGCTTTCGGCTACTACTCAGAAAATTTCTTGAGATTTTGCGA |
| | | ATGTTATGCGACTGAAGGGGTCAATATATATAAGGTAAAACATCCCATCACCCAGGA |
| | | GTGGTTCGAGGCCTCTAGGGATCGAGACGGT 3′ |
| 95 | 2551 | 5′ |
| | | CTTCCTTGAAACTTGCCTATGATCGTAGGAGTCTTAGTAAGGATAAAGAAACCGTT |
| | | GCGTGGGTGCGTAAGACCCTTTCTAAATAATGTTGGTCACATTTAAGACTTGTTTA |
| | | GTCCACATTAGGACTGGTTCTAACAGTTTCTTTAAACTGTAATCGTCGTTGCGACG |
| | | TTGGTTTGCTTACAAGCAATCAAGCTGCCTTTG 3′ |
| 96 | 2594 | 5′ |
| | | TAAAGAAACCGTTGCGTGGGTGCGTAAGACCCTTTCTAAATAATGTTGGTCACATT |
| | | TAAGACTTGTTTAGTCCACATTAGGACTGGTTCTAACAGTTTCTTTAAACTGTAATC |
| | | GTCGTTGCGACGTTGGTTTGCTTACAAGCAATCAAGCTGCCTTTGAGTTTTACTCC |
| | | TTGAACTCTTCAGAAGAATTCTTCGGAATTCG 3′ |
| 97 | 2676 | 5′ |
| | | CTGGTTCTAACAGTTTCTTTAAACTGTAATCGTCGTTGCGACGTTGGTTTGCTTAC |
| | | AAGCAATCAAGCTGCCTTTGAGTTTTACTCCTTGAACTCTTCAGAAGAATTCTTCG |
| | | GAATTCGTACCAGTATCTCACATAGTGAGGTAATAAGACTGGTGGGCAGCGCCTAG |
| | | TCGAAAGACTAGGTGATCTCTAAGGAGACCA 3′ |

TABLE 8
Sequences containing packaging signals of CCMV3 PSs.

| SEQ | | |
|---|---|---|
| ID | | |
| NO: | | |
| 98 | 221 | 5′ |
| | | TAACGCTAAACCGTACCATAGTAGGCTGTTACCTGACTCGAACTCAGGCGGACGT |
| | | CAGCTGACATTCACGGAATAGTTCGATATCATAATTCCTCGTTCTTTGCTGTTATAG |
| | | CTCCCGATGTCTAACACTACTTTTAGACCTTTTACTGGTTCCTCCAGGACCGTGGT |
| | | CGAGGGAGAACAAGCCGGCGCCCAGGATGATAT 3′ |
| 99 | 341 | 5′ |
| | | GTCTAACACTACTTTTAGACCTTTTACTGGTTCCTCCAGGACCGTGGTCGAGGGA |
| | | GAACAAGCCGGCGCCCAGGATGATATGTCGTTGTTACAGTCACTTTTTTCCGACA |
| | | AATCCAGGGAGGAGTTTGCTAAGGAGTGTAAGTTGGGTATGTATACCAATTTATCC |
| | | TCTAATAACCGGCTTAATTATATAGATCTAGTCCC 3′ |
| 100 | 547 | 5′ |
| | | ACACTGGTAGTAGAGCTCTGAACTTATTTAAGTCAGAGTATGAAAAAGGTCACATT |
| | | CCCTCCAGCGGTGTGCTTAGTATACCTAGAGTGCTGGTTTTTCTTGTGAGGACGA |
| | | CAACAGTGACTGAATCTGGGAGTGTCACCATTAGATTGGTTGACTTGATAAGCGCT |
| | | TCGTCGGTTGAGATTTTAGAACCTGTGGATGGTA 3′ |
| 101 | 221 | 5′ |
| | | TAACGCTAAACCGTACCATAGTAGGCTGTTACCTGACTCGAACTCAGGCGGACGT |
| | | CAGCTGACATTCACGGAATAGTTCGATATCATAATTCCTCGTTCTTTGCTGTTATAG |
| | | CTCCCGATGTCTAACACTACTTTTAGACCTTTTACTGGTTCCTCCAGGACCGTGGT |
| | | CGAGGGAGAACAAGCCGGCGCCCAGGATGATAT 3′ |
| 102 | 607 | 5′ |
| | | CCAGCGGTGTGCTTAGTATACCTAGAGTGCTGGTTTTTCTTGTGAGGACGACAAC |
| | | AGTGACTGAATCTGGGAGTGTCACCATTAGATTGGTTGACTTGATAAGCGCTTCGT |
| | | CGGTTGAGATTTTAGAACCTGTGGATGGTACGCAAGAGGCTACTATTCCTATTTCT |
| | | AGTCTTCCGGCTATCGTTTGTTTTTCTCCTAGTT 3′ |
| 103 | 697 | 5′ |
| | | ′TTGACTTGATAAGCGCTTCGTCGGTTGAGATTTTAGAACCTGTGGATGGTACGCA |
| | | AGAGGCTACTATTCCTATTTCTAGTCTTCCGGCTATCGTTTGTTTTTCTCCTAGTTAT |
| | | GACTGTCCCATGCAGATGATAGGGAATAGACACAGATGTTTCGGTTTGGTAACTCA |
| | | ACTGGATGGTGTCATATCCTCAGGGTCTACCG 3′ |
| 104 | 826 | 5′TGATAGGGAATAGACACAGATGTTTCGGTTTGGTAACTCAACTGGATGGTGTCAT |
| | | ATCCTCAGGGTCTACCGTCGTTATGAGTCATGCGTATTGGTCTGCGAACTTTCGTA |
| | | GTAAACCTAATAACTACAAGCAGTACGCACCTATGTATAAGTATGTGGAACCCTTTG |
| | | ACAGGTTGAAACGTTTGAGCCGTAAACAATTGA 3′ |
| 105 | 1328 | 5′ |
| | | GAACCCGCCGAAAGGACAGGCTGAGGGCGTACGATTCATGTGTAGCTGGCTGGG |
| | | TGTGAGACACAAAAAAAAAAAAAAAAAAAAAAAAAAAAAAAAAAAAAAAATCTATGT |
| | | TTAATTTGATAGTAATTTATCATGTCTACAGTCGGAACAGGGAAGTTAACTCGTGCA |
| | | CAACGAAGGGCTGCGGCCCGTAAGAACAAGCGG 3′ |
| 106 | 1641 | 5′ |
| | | CTGCCGAAGCTAAAGTAACCTCGGCTATAACTATCTCTCTCCCTAATGAGCTATCGT |
| | | CCGAAAGGAACAAGCAGCTCAAGGTAGGTAGAGTTTTATTATGGCTTGGGTTGCT |
| | | TCCCAGTGTTAGTGGCACAGTGAAATCCTGTGTTACAGAGACGCAGACTACTGCT |
| | | GCTGCCTCCTTTCAGGTGGCATTAGCTGTGGCCG 3′ |
| 107 | 2000 | 5′ |
| | | ACGTTTGACGACTCTTTCACTCCGGTGTATTAGTGCCCGCTGAAGAGCGTTACAC |
| | | TAGTGTGGCCTACTTGAAGGCTAGTTATAACCGTTTCTTTAAACGGTAATCGTTGTT |
| | | GAAACGTCTTCCTTTTACAAGAGGATTGAGCTGCCCTTGGGTTTTACTCCTTGAAC |
| | | CCTTCGGAAGAACTCTTTGGAGTTCGTACCAGT 3′ |

TABLE 9
CCMV aptamers identified via SELEX

| SEQ ID NO: | Aptamer N | Sequence |
|---|---|---|
| 108 | 1 | 5′ GAUUAUGUGUCUCUUUCUA |
| | | AUUGGUUUUAACACGGUUUC 3′ |
| 109 | 2 | 5′ CUGUAGAAAUUGGUUUUCU |
| | | UUCAG 3′ |
| 110 | 3 | 5′ CGUACGUUUCUCUUCGAAA |
| | | UUUCG 3′ |
| 111 | 4 | 5′ UCAACGCACUUUUAUUUGG |
| | | CAACGUGA 3′ |
| 112 | 5 | 5′ GCGUCAACAACGGUUUUC |
| | | UCGUUUUCCUUACGU 3′ |
| 113 | 6 | 5′ UUUCGUUUCGUCUUCCUAA |
| | | AUUUAAA 3′ |

### Brome Mosaic Virus (BMV):

TABLE 10
Sequences containing BMV1 packaging signals

| SEQ | | |
|---|---|---|
| ID | | |
| NO: | | Sequence |
| 114 | 52 | 5′ |
| | | GTAGACCACGGAACGAGGTTCAATCCCTTGTCGACCACGGTTCTGCTACTTG |
| | | TTCTTTGTTTTTCACCAACAAAATGTCAAGTTCTATCGATTTGCTGAAGTTGAT |
| | | TGCTGAGAAGGGTGCTGACAGCCAGAGTGCCCAAGACATCGTAGAC 3′ |
| 115 | 545 | 5′ |
| | | GTTGTTGTCCTGTGTTGGGTGTTAGAGACGCTGCCCGACATGAGGAGAGGA |
| | | TGTGCCGCATGCGAAAAATTTTGCAAGAAAGCGATGATTTCGATGAAGTCCC |
| | | GAACTTTTGTCTTAACCGAGCTCAAGATTGTGATGTCCAAGCTGATTGGGCTA |
| | | TCTGTATCCACGGCGGTTATGATATGGGCTTCCAAGGTCTGTGTG 3′ |
| 116 | 736 | 5′ |
| | | GGTCTGTGTGACGCCATGCATTCGCATGGAGTACGCGTACTACGTGGTACCG |
| | | TTATGTTCGACGGCGCCATGTTGTTTGACCGCGAGGGTTTTCTTCCCTTGCTT |
| | | AAATGTCACTGGCAACGTGACGGGTCAGGCGCGGATGAGGTGATCAAATTC |
| | | GATTTTGAAAATGAAAGCACATTATCTTACATCCACGGATGGCAA 3′ |
| 117 | 1265 | 5′ |
| | | TATCCGCCAAGTCGTCGACTGTTATTATTAACGGTCAGGCTATCATGGCTGGT |
| | | GAGCGCTTAGACATTGAAGATTATCATCTAGTGGCCTTTGCTTTGACTTTGAAT |
| | | CTGTATCAAAAGTACGAAAAGCTTACGGCCCTCCGCGATGGGATGGAATGGA |
| | | AAGGTTGGTGCCATCACTTCAAAACTAGGTTTTGGTGGGGTG 3′ |
| 118 | 1462 | 5′ |
| | | GGTGGAGATTCATCCAGGGCGAAAGTAGGATGGCTGAGAACATTGGCTAGC |
| | | AGATTTCCCCTACTACGTCTGGATTCTTATGCGGACAGTTTTAAGTTTCTGACT |
| | | CGTCTCTCAAACGTTGAAGAATTTGAGCAAGATTCTGTACCGATATCACGTTT |
| | | GAGAACGTTTTGGACTGAAGAGGACTTATTCGACCGGCTGGAG 3′ |
| 119 | 2854 | 5′ |
| | | TGGATTGATGGACACATAAAAACAGTACACGAAGCGCAAGGGATCTCTGTTG |
| | | ACAACGTCACTTTGGTTCGGCTTAAGTCGACCAAATGTGATTTGTTTAAACAT |
| | | GAGGAGTACTGTTTGGTTGCCTTAACACGACACAAGAAGTCCTTTGAGTATT |
| | | GCTTTAACGGCGAGCTCGCTGGTGATTTGATCTTTAATTGTGTT 3′ |
| 120 | 2952 | 5′ |
| | | TAAACATGAGGAGTACTGTTTGGTTGCCTTAACACGACACAAGAAGTCCTTTG |
| | | AGTATTGCTTTAACGGCGAGCTCGCTGGTGATTTGATCTTTAATTGTGTTAAGT |
| | | GATGCGCTTGTCTCTGTGTGAGACCTCTGCTCGAGGAGAGCCCTGTTCCAG |
| | | GTAGGAACGTTGTGGTCTAACTCAAGACTAGCTGAATCGGTGC 3′ |
| 121 | 3131 | 5′ |
| | | TCAAGACTAGCTGAATCGGTGCTATAACCGATAGTCGTGGTTGACACGCAGA |
| | | CCTCTTACAAGAGTGTCTAGGCGCCTTTGAGAGTTACTCTTTGCTCTCTTCG |
| | | GAAGAACCCTTAGGGGTTCGTGCATGGGCTTGCATAGCAAGTCTTAGAATGC |
| | | GGGTGTCGTACAGTGTTGAAAAACACTGTAAATCTCTAAAAGAGA 3′ |

TABLE 11
Sequences containing BMV2 packaging signals

| SEQ | | |
|---|---|---|
| ID | | |
| NO: | | Sequence |
| 122 | 87 | 5′ |
| | | GTAAACCACGGAACGAGGTTCAATCCCTTGTCGACCCACGGTTTGCGCAAC |
| | | ACACATCTGACCTTGTTGTTGTTGTGTGCTTGTTCTTTCTACTATCACCAAGAT |
| | | GTCTTCGAAAACCTGGGATGATGATTTCGTTCGCCAGGTCCCGTCTTTCCAA |
| | | TGGATCATAGATCAATCCTTAGAAGACGAG 3′ |
| 123 | 1380 | 5′ |
| | | CCTGTTGTAACTGACACCCTTCACTTGGAACGAGCAGTAGCAGCTACTATAAC |
| | | ATTTCATAGTAAAGGTGTGACTAGTAATTTTTCACCCTTTTTCACTGCTTGTTT |
| | | CGAGAAGTTATCACTGGCCCTGAAATCCAGGTTCATTGTGCCTATCGGAAAG |
| | | ATATCCTCTCTGGAGCTTAAGAATGTCCGCTTGAATAACAGA 3′ |
| 124 | 1620 | 5′ |
| | | CAGGGTGAGCTGCACCTAGAGTTTCAGAGAGAGATACTCCTTGCGCTGGGC |
| | | TTTCCAGCGCCGCTGACGAATTGGTGGTCTGATTTTCATCGCGATTCTTATTT |
| | | ATCAGACCCTCATGCCAAGGTGGGAATGTCCGTTTCCTTCCAACGCAGAACT |
| | | GGTGACGCGTTTACATATTTCGGTAATACTCTTGTCACTATGGCT 3′ |
| 125 | 1788 | 5′ |
| | | ACATATTTCGGTAATACTCTTGTCACTATGGCTATGATTGCATATGCCTCTGATC |
| | | TAAGTGACTGTGACTGTGCAATATTTTCAGGAGATGATTCTTTAATCATCTCTA |
| | | AAGTTAAGCCAGTCCTGGATACCGATATGTTTACGTCTCTCTTCAATATGGAGA |
| | | TAAAAGTCATGGACCCTAGTGTGCCCTACGTTTGTAGT 3′ |
| 126 | 2012 | 5′ |
| | | GGGCAATTTGGTGTCTGTACCAGATCCTCTGAGAGAGATCCAGCGCTTAGCT |
| | | AAGCGAAAGATTCTGCGTGATGAACAGATGCTCAGAGCACATTTCGTTTCCT |
| | | TCTGTGATCGAATGAAGTTTATTAATCAACTTGATGAGAAGATGATTACGACGC |
| | | TCTGTCATTTTGTTTATCTGAAATATGGGAAAGAAAAACCTTG 3′ |
| 127 | 2079 | 5′ |
| | | CGTGATGAACAGATGCTCAGAGCACATTTCGTTTCCTTCTGTGATCGAATGAA |
| | | GTTTATTAATCAACTTGATGAGAAGATGATTACGACGCTCTGTCATTTTGTTTAT |
| | | CTGAAATATGGGAAAGAAAAACCTTGGATTTTCGAGGAGGTTAGAGCTGCTC |
| | | TTGCGGCTTTTTCTTTATACTCCGAGAATTTCCTGAGGTTC 3′ |
| 128 | 2163 | 5′ |
| | | ACGACGCTCTGTCATTTTGTTTATCTGAAATATGGGAAAGAAAAACCTTGGAT |
| | | TTTCGAGGAGGTTAGAGCTGCTCTTGCGGCTTTTTCTTTATACTCCGAGAATT |
| | | TCCTGAGGTTCTCTGATTGCTACTGTACCGAAGGCATCAGAGTTTATCAGATG |
| | | AGCGATCCTGTATGTAAGTTCAAACGCACCACGGAAGAGCGT 3′ |
| 129 | 2762 | 5′ |
| | | TAAAAGCTTGTTGAATCAGTACAATAACTGATAGTCGTGGTTGACACGCAGAC |
| | | CTCTTACAAGAGTGTCTAGGTGCCTTTGAGAGTTACTCTTTGCTCTCTTCGGA |
| | | AGAACCCTTAGGGGTTCGTGCATGGGCTTGCATAGCAAGTCTTAGAATGCGG |
| | | GTGCCGTACAGTGTTGAAAAACACTGTAAATCTCTAAAAGAGA 3′ |

TABLE 12
Sequences containing BMV3 packaging signals

| SEQ | | |
|---|---|---|
| ID | | |
| NO: | | Sequence |
| 130 | 68 | 5′ |
| | | GTAAAATACCAACTAATTCTCGTTCGATTCCGGCGAACATTCTATTTTACCAAC |
| | | ATCGGTTTTTTCAGTAGTGATACTGTTTTTGTTCCCGATGTCTAACATAGTTTC |
| | | TCCCTTCAGTGGTTCCTCACGAACTACGTCTGACGTTGGCAAGCAAGCGGG |
| | | AGGTACTAG 3′ |
| 131 | 400 | 5′ |
| | | CACACGTATCTGCTTGGCTCTCATGGGCTACATCCAAGTATGATAAAGGAGAG |
| | | TTACCTTCCAGGGGATTCATGAACGTTCCACGCATCGTTTGTTTTCTCGTTCG |
| | | TACCACAGATAGCGCAGAGTCCGGTTCTATAACCGTGAGCCTGTGCGATTCT |
| | | GGTAAGGCTGCTCGTGCTGGAGTACTCGAAGCCATTGATAATC 3′ |
| 132 | 1106 | 5′ |
| | | AAATCCGGTCTAACAAGCTCGGTCCATTTCGTAGAGTTAAGCAAGCTGGGGA |
| | | GACCCCCGACAGCCGTTTGGATCAGCGCTCGCGTCTCGTTTGGGTTCAATT |
| | | CCCTTACCTTACAACGGCGTGTTGAGATAGGTCCTCGGGGGAGGTTATCCAT |
| | | GTTTGTGGATATTCTATGTTGTGTGTCTGAGTTATTATTAAAAAAA 3′ |
| 133 | 1172 | 5′ |
| | | GTTTGGATCAGCGCTCGCGTCTCGTTTGGGTTCAATTCCCTTACCTTACAAC |
| | | GGCGTGTTGAGATAGGTCCTCGGGGGAGGTTATCCATGTTTGTGGATATTCTA |
| | | TGTTGTGTGTCTGAGTTATTATTAAAAAAAAAAAAAAAAGATCTATGTCCTAATT |
| | | CAGCGTATTAATAATGTCGACTTCAGGAACTGGTAAGATGA 3′ |
| 134 | 1200 | 5′ |
| | | GGTTCAATTCCCTTACCTTACAACGGCGTGTTGAGATAGGTCCTCGGGGGAG |
| | | GTTATCCATGTTTGTGGATATTCTATGTTGTGTGTCTGAGTTATTATTAAAAAAA |
| | | AAAAAAAAAGATCTATGTCCTAATTCAGCGTATTAATAATGTCGACTTCAGGAA |
| | | CTGGTAAGATGACTCGCGCGCAGCGTCGTGCTGCCGCTCG 3′ |
| 135 | 2005 | 5′ |
| | | GGTTAAAAGCTTGTTGAATCAGTACAATAACTGATAGTCGTGGTTGACACGCA |
| | | GACCTCTTACAAGAGTGTCTAGGTGCCTTTGAGAGTTACTCTTTGCTCTCTTC |
| | | GGAAGAACCCTTAGGGGTTCGTGCATGGGCTTGCATAGCAAGTCTTAGAATG |
| | | CGGGTACCGTACAGTGTTGAAAAACACTGTAAATCTCTAAAAG 3′ |

TABLE 13
Capsid Protein binding sites

| SEQ | | |
|---|---|---|
| ID | | Sequence |
| 136 | TCVCP | MENDPRVRKFASDGAQWAIKWQKKGWSTLTSRQKQTARAAMGIKLSPVAQPVQ |
| | | KVTRLSAPVALAYREVSTQPRVSTARDGITRSGSELITTLKKNTDTEPKYTTAVLN |
| | | PSEPGTFNQLIKEAAQYEKYRFTSLRFRYSPMSPSTTGGKVALAFDRDAAKPPP |
| | | NDLASLYNIEGCVSSVPWTGFILTVPTDSTDRFVADGISDPKLVDFGKLIMATYGQ |
| | | GANDAAQLGEVRVEYTVQLKNRTGSTSDAQIGQFAGVKDGPRLVSWSKTKGTA |
| | | GWEHDCHFLGTGNFSLTLFYEKAPVSGLENADASDFSVLGEAAAGSVQWAGVK |
| | | VAERGQGVKMVTTEEQPKGKLQALRI |
| 137 | HPeVV0-3 | METIKSIADMATGVVSSVDSTINAVNEKVESVGNEIGGNLLTKVADDASNILGPNC |
| | | FATTAEPENKNVVQATTTVNTTNLTQHPSAPTMPFSPDFSNVDNFHSMAYDITTG |
| | | DKNPSKLVRLETHEWTPSWARGYQITHVELPKVFWDHQDKPAYGQSRYFAAVR |
| | | CGFHFQVQVNVNQGTAGSALVVYEPKPVVTYDSKLEFGAFTNLPHVLMNLAETT |
| | | QADLCIPYVADTNYVKTDSSDLGQLKVYVWTPLSIPTGSANQVDVTILGSLLQLD |
| | | FQNPRVFAQDVNIYDNAPNGKKKNWKKIMTMSTKYKWTRTKIDIAEGPGSMNM |
| | | ANVLCTTGAQSVALVGERAFYDPRTAGSKSRFDDLVKIAQLFSVMADSTTPSEN |
| | | HGVDAKGYFKWSATTAPQSIVHRNIVYLRLFPNLNVFVNSYSYFRGSLVLRLSVY |
| | | ASTFNRGRLRMGFFPNATTDSTSTLDNAIYTICDIGSDNSFEITIPYSFSTWMRKT |
| | | NGHPIGLFQIEVLNRLTYNSSSPSEVYCIVQGKMGQDARFFCPTGSVVTFQNSW |
| | | GSQMDLTDPLCIEDDTENCKQTMSPNELGLTSAQDDGPLGQEKPNYFLNFRSM |
| | | NVDIFTVSHTKVDNLFGRAWFFMEHTFTNEGQWRVPLEFPKQGHGSLSLLFAYF |
| | | TGELNIHVLFLSERGFLRVAHTYDTSNDRVNFLSSNGVITVPAGEQMTLSAPYYS |
| | | NKPLRTVRDNNSLGYLMCKPFLTGTSTGKIEVYLSLRCPNFFFPLPAPKVTSSRA |
| | | LRGDMANL |
| 138 | CCMVCP | MSTVGTGKLTRAQRRAAARKNKRNTRVVQPVIVEPIASGQGKAIKAWTGYSVSK |
| | | WTASCAAAEAKVTSAITISLPNELSSERNKQLKVGRVLLWLGLLPSVSGTVKSCV |
| | | TETQTTAAASFQVALAVADNSKDVVAAMYPEAFKGITLEQLAADLTIYLYSSAALTE |
| | | GDVIVHLEVEHVRPTFDDSFTPVY |
| 139 | BMVCP | MSTSGTGKMTRAQRRAAARRNRWTARVQPVIVEPLAAGQGKAIKAIAGYSISKW |
| | | EASSDAITAKATNAMSITLPHELSSEKNKELKVGRVLLWLGLLPSVAGRIKACVAE |
| | | KQAQAEAAFQVALAVADSSKEVVAAMYTDAFRGATLGDLLNLQIYLYASEAVPAK |
| | | AVVVHLEVEHVRPTFDDFFTPVYR |
| 140 | HIV NC | AEAMSQVTNPATIMIQKGNFRNQRKTVKCFNCGKEGHIAKNCRAPRKKGCWKC |
| | | GKEGHQMKDCTERQANFLGKIWPSHKGRPGNF |
| 141 | HIV CA | PRTLNAWVKVVEEKAFSPEVIPMFSALSEGATPQDLNTMLNTVGGHQAAMQML |
| | | KETINEEAAEWDRLHPVHAGPIAPGQMREPRGSDIAGTTSTLQEQIGWMTHNPP |
| | | IPVGEIYKRWIILGLNKIVRMYSPTSILDIRQGPKEPFRDYVDRFYKTLRAEQASQE |
| | | VKNWMTETLLVQNANPDCKTILKALGPGATLEEMMTACQGVGGPGHKARVL |

TABLE 14
PS sequences for STNV-1

| SEQ | | | |
|---|---|---|---|
| ID NO: | Start | End | Sequence |
| 476 | 1 | 22 | 5′ AGUAAAGACAGGAAACUUUACU 3′ |
| 477 | 38 | 54 | 5′ ACAACAGAACAACAGGC 3′ |
| 478 | 62 | 73 | 5′ CGCAACAAUGCG 3′ |
| 479 | 88 | 100 | 5′ UGAUAAAUACACA 3′ |
| 480 | 107 | 121 | 5′ GCAUAAAAGGUUUGC 3′ |
| 481 | 133 | 147 | 5′ CAGGGAACACCAAUG 3′ |
| 482 | 159 | 174 | 5′ ACAGUACAAAAUCUGU 3′ |
| 483 | 183 | 200 | 5′ AUAAUCCAAGGAGAUGAU 3′ |
| 484 | 203 | 219 | 5′ CAACCAGAGAAGUGGUG 3′ |
| 485 | 249 | 264 | 5′ CACGUACGAGGCACUG 3′ |
| 486 | 301 | 316 | 5′ UUCGUGAUAACAUGAA 3′ |
| 487 | 319 | 334 | 5′ GUGGGACCACUCCCAC 3′ |
| 488 | 346 | 359 | 5′ UGUUGAACACUGCG 3′ |
| 489 | 375 | 394 | 5′ UAUAACCCAAUCACGUUGCA 3′ |
| 490 | 412 | 425 | 5′ UACUCAAGGAUGUA 3′ |
| 491 | 461 | 478 | 5′ AGAUCGGAUAAUUAACCU 3′ |
| 492 | 480 | 492 | 5′ CCAGGACAACUGG 3′ |
| 493 | 512 | 527 | 5′ GGCUGUAGCAGCCUCC 3′ |
| 494 | 650 | 665 | 5′ GCGCUGAAAGAUGCGU 3′ |
| 495 | 696 | 709 | 5′ UAAGCAGAAAUCCA 3′ |
| 496 | 725 | 744 | 5′ GGUGGAAAGCAGUCCCAGCU 3′ |
| 497 | 804 | 822 | 5′ UAGUCUAAAUGAGACGUUG 3′ |
| 498 | 914 | 930 | 5′ UGCCAUUAGUAGGUCUA 3′ |
| 499 | 962 | 980 | 5′ UGCAACAAGAAUAUGUGCG 3′ |
| 500 | 996 | 1013 | 5′ GCGGUAUAUUAAGUGCGC 3′ |
| 501 | 1026 | 1039 | 5′ GUUUGGACCAGGGC 3′ |
| 502 | 1083 | 1097 | 5′ GCUUUAGGAGAUGAU 3′ |
| 503 | 1101 | 1121 | 5′ GUAUAGUUAUUAGACAAAUGC 3′ |
| 504 | 1155 | 1175 | 5′ GGCCAAGCGAAGAACCUCAUC 3′ |
| 505 | 1196 | 1217 | 5′ AAAUUUGGUACCAUCCAAACUU 3′ |

TABLE 15
PS sequences for STNV-2

| SEQ ID NO: | | | Sequence |
|---|---|---|---|
| 506 | 5 | 19 | 5′ AAGACAGGAAACUUU 3′ |
| 507 | 34 | 46 | 5′ UGACAAAACGUCA 3′ |
| 508 | 60 | 74 | 5′ AACCGCAAGAGCGUU 3′ |
| 509 | 85 | 99 | 5′ UGCGUAGUAUUGUUG 3′ |
| 510 | 111 | 124 | 5′ GAGCAGAAGCGAUU 3′ |
| 511 | 134 | 147 | 5′ UACGAACACCAACA 3′ |
| 512 | 150 | 172 | 5′ GUCACUACAGCAGGUACCGUGAU 3′ |
| 513 | 175 | 188 | 5′ ACCUGAGCAACAAC 3′ |
| 514 | 194 | 211 | 5′ GCAAGGAGAUGACCUUGU 3′ |
| 515 | 230 | 246 | 5′ GAUUAAGACCAUACACC 3′ |
| 516 | 262 | 278 | 5′ GGUGUACAGGAAUUACC 3′ |
| 517 | 307 | 322 | 5′ UUCGUGACAACACCAA 3′ |
| 518 | 326 | 341 | 5′ GGGGACUACACCGGCU 3′ |
| 519 | 361 | 383 | 5′ GUGCUAGUAUAACAUCCCAGUAU 3′ |
| 520 | 399 | 417 | 5′ CAGCAAAAGAGGUUCACUG 3′ |
| 521 | 476 | 496 | 5′ UGCCGUUGAUAAGAAACGGCG 3′ |
| 522 | 498 | 520 | 5′ GCGAUAUUUUACAACGGUGCUGC 3′ |
| 523 | 566 | 579 | 5′ CAUUGGAUCACAUG 3′ |
| 524 | 583 | 603 | 5′ CUGGACAGUAUGAUGUGACAG 3′ |
| 525 | 637 | 654 | 5′ UCAUGAUGAUGAUAGUGA 3′ |
| 526 | 658 | 673 | 5′ ACGCUGAAAGAUGCGU 3′ |
| 527 | 734 | 745 | 5′ GGACAGUAGUCC 3′ |
| 528 | 748 | 759 | 5′ AACUAGUAAAUC 3′ |
| 529 | 762 | 780 | 5′ GACCGGGAGAAAACCAGCU 3′ |
| 530 | 812 | 828 | 5′ GUGGAACGAGGCCCCGC 3′ |
| 531 | 852 | 863 | 5′ GUGGAAAACCAU 3′ |
| 532 | 909 | 923 | 5′ GUGCAACAAUGCUGU 3′ |
| 533 | 938 | 953 | 5′ CUCAACAUCACUUCAA 3′ |
| 534 | 964 | 976 | 5′ AUGUCACAAGAAU 3′ |
| 535 | 1105 | 1125 | 5′ GUAUAGUGACUAGACAAAUGC 3′ |
| 536 | 1173 | 1185 | 5′ GCCUCAACAAGGU 3′ |
| 537 | 1194 | 1208 | 5′ UGCAUAGGAGAUGUG 3′ |

TABLE 16
PS sequences for STNV-c

| SEQ ID NO: | | | Sequence |
|---|---|---|---|
| 538 | 20 | 32 | 5′ UUAUACAAAGUAG 3′ |
| 539 | 34 | 53 | 5′ UCAUGGUAUUAGGGUGGUGG 3′ |
| 540 | 64 | 75 | 5′ CUGAAAGAUUAA 3′ |
| 541 | 99 | 114 | 5′ AACAUGACUAAACGUC 3′ |
| 542 | 125 | 142 | 5′ ACAAACAACUAGAUCUGU 3′ |
| 543 | 140 | 155 | 5′ UGUUAGAUCACUCACG 3′ |
| 544 | 163 | 182 | 5′ ACGUGCGGAACAUCAUACGU 3′ |
| 545 | 195 | 208 | 5′ ACCAAACGAUUUGU 3′ |
| 546 | 227 | 245 | 5′ UCUUAACAGUACCGCUGGA 3′ |
| 547 | 269 | 285 | 5′ CAUCAUACAAGGCGAUG 3′ |
| 548 | 302 | 318 | 5′ UGGAGAUAAGAUUCGUA 3′ |
| 549 | 344 | 363 | 5′ AGCGACUGCCAUAACAAAUU 3′ |
| 550 | 386 | 400 | 5′ GUUUAAGGAUAACAC 3′ |
| 551 | 404 | 420 | 5′ UCGUGGUACCACUCCAA 3′ |
| 552 | 425 | 441 | 5′ GACUGAAGUACUUAACU 3′ |
| 553 | 455 | 468 | 5′ GGCCCAAUACAACC 3′ |
| 554 | 479 | 492 | 5′ ACUACAGCAUAGGU 3′ |
| 555 | 499 | 514 | 5′ UCCUCAAGGAUGUUGA 3′ |
| 556 | 528 | 541 | 5′ CUGUCAGGAGAGAG 3′ |
| 557 | 552 | 568 | 5′ UUGGUGAUGACGCAUGG 3′ |
| 558 | 580 | 595 | 5′ GUUUCUAUAAUGGAAC 3′ |
| 559 | 624 | 636 | 5′ GGAGCAAUAUUCC 3′ |
| 560 | 678 | 689 | 5′ GGUUACGAGGCU 3′ |
| 561 | 795 | 812 | 5′ UUUGAAAAAUCAUUCAAA 3′ |
| 562 | 812 | 825 | 5′ AUGUCACCAGACGU 3′ |
| 563 | 826 | 843 | 5′ AUCCCUGAACCAGGCUGU 3′ |
| 564 | 878 | 893 | 5′ CUGCUAGGACGAAUGG 3′ |
| 565 | 904 | 919 | 5′ UAAUACACAAGGUUCG 3′ |
| 566 | 923 | 937 | 5′ AUAGUAGGAAGCCGU 3′ |
| 567 | 957 | 974 | 5′ GGUAAUUUACGAAAGACC 3′ |
| 568 | 1003 | 1018 | 5′ UUCUGGCAUAAUUGAG 3′ |
| 569 | 1056 | 1072 | 5′ GAUAAAAGGAGUUGAUC 3′ |
| 570 | 1119 | 1133 | 5′ UGUGGAAGAAUUCUG 3′ |
| 571 | 1159 | 1176 | 5′ GGGGAGUACUACACCUUC 3′ |
| 572 | 1182 | 1195 | 5′ CACUAAGGACUAUG 3′ |

TABLE 17
Sequences for HPeV PS (FIG. 1E)

| SEQ | | | | |
|---|---|---|---|---|
| ID | | | | |
| No. | | Start | End | Sequence |
| 578 | PS1 | 340 | 360 | UAAAAUGUCUGGUGAGAUGUG |
| 579 | PS2 | 676 | 714 | UCCCUGGUUUCCUUUUAUUGUUAAUAU |
| | | | | UGACAUUAUGGA |
| 580 | PS3 | 746 | 772 | GGUGUUGUAAGUUCUGUUGAUUCUACC |
| 581 | PS4 | 815 | 840 | AUUGGAGGUAAUUUGUUAACUAAAGU |
| 582 | PS5 | 1129 | 1150 | AUUACCUAAAGUUUUUUGGGAU |
| 583 | PS6 | 1329 | 1347 | UUCCACAUGUUUUGAUGAA |
| 584 | PS7 | 1950 | 1971 | UGAAUGUUUUUGUUAACAGUUA |
| 585 | PS8 | 1985 | 2004 | GGUUCAUUAGUUUUAAGAUU |
| 586 | PS9 | 2313 | 2335 | CUGGUUCUGUUGUUACAUUCCAG |
| 587 | PS10 | 2484 | 2505 | UUCUCAAUUUUAGGUCGAUGAA |
| 588 | PS11 | 2642 | 2673 | UUAUCACUGUUGUUUGCUUAUUUUACU |
| | | | | GGUGA |
| 589 | PS12 | 2864 | 2891 | AGUCUUGGUUAUUUGAUGUGCAAGCCCU |
| 590 | PS13 | 2919 | 2940 | UUGAGGUUUAUCUUAGCCUGAG |
| 591 | PS14 | 3540 | 3563 | UGGAUAAUGAUUUAGUCAAGUUCA |
| 592 | PS15 | 4028 | 4044 | GACAUUAUUGUUGAGUC |
| 593 | PS16 | 4332 | 4350 | UUAAUGGUGUUUUUACUAA |
| 594 | PS17 | 5060 | 5084 | UCCAUGCUCAGUUUUGUUGAGAGGA |
| 595 | PS18 | 5127 | 5151 | UUAGUAUACUUUUGUUGGUAACAAA |
| 596 | PS19 | 6181 | 6209 | AGCUGGUUAUAGUUUUGUUAAAUCUGG |
| | | | | CU |
| 597 | PS20 | 6397 | 6426 | UUGUGAAGUUGAUUAUUGCAUUGUUUA |
| | | | | CAG |
| 598 | PS21 | 6777 | 6796 | UGAUGUGUAUUUACACUACA |
| 599 | PS22 | 7251 | 7273 | AAGAUUAAUGUUUUGUUUUUCUU |
| 600 | PS9′ | | | CUGGAAGUGUAGUAACAUUCCAG |
| 601 | PS22′ | | | AAGACGAAUGAAACGUUCGUCUU |

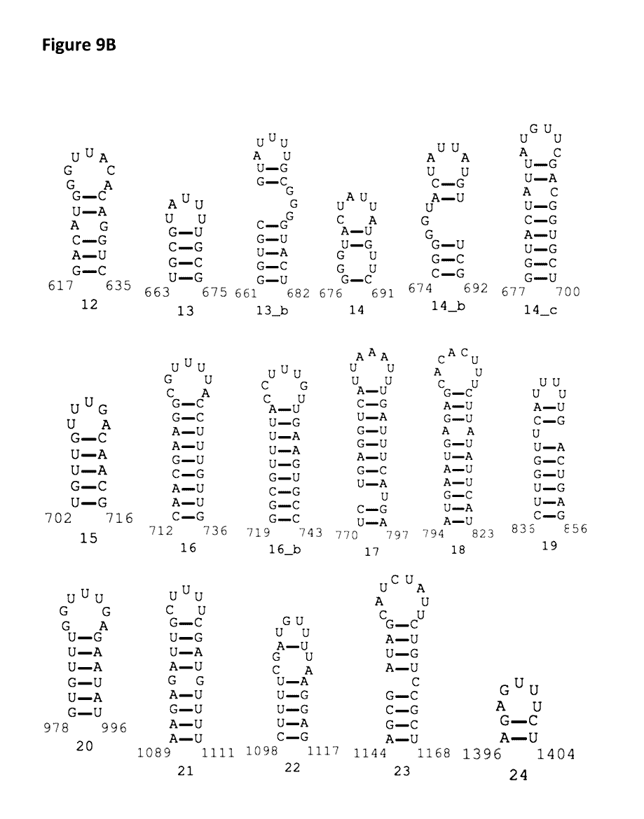

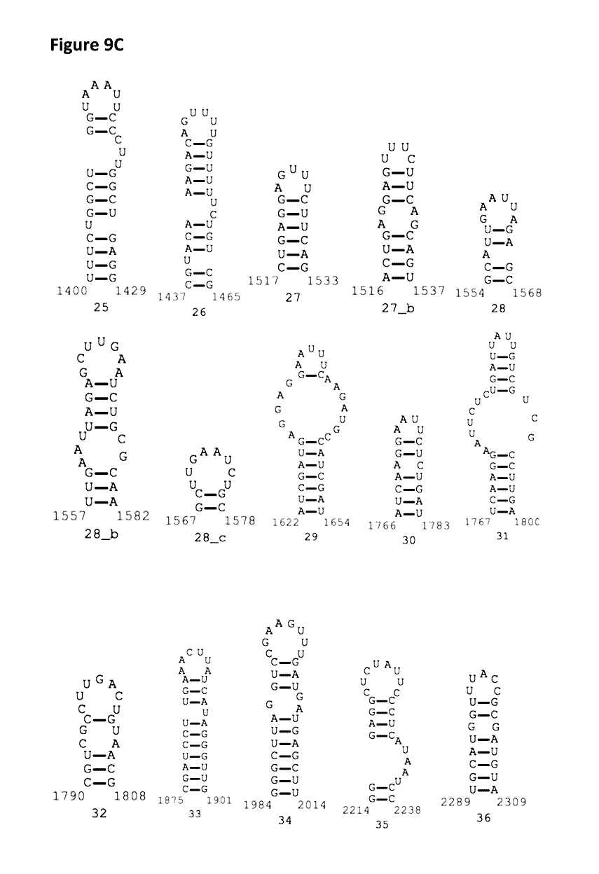

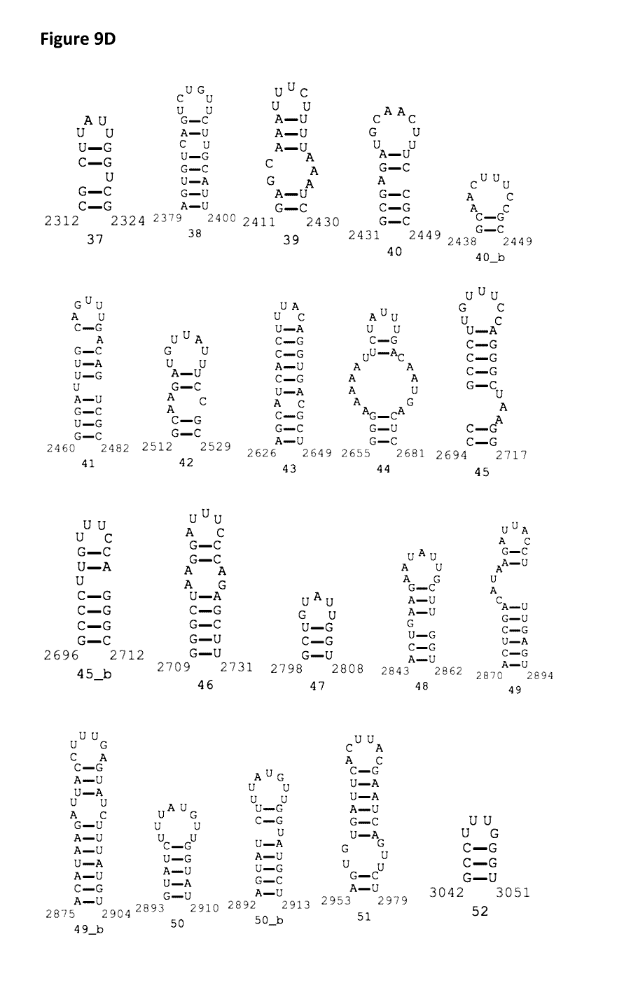

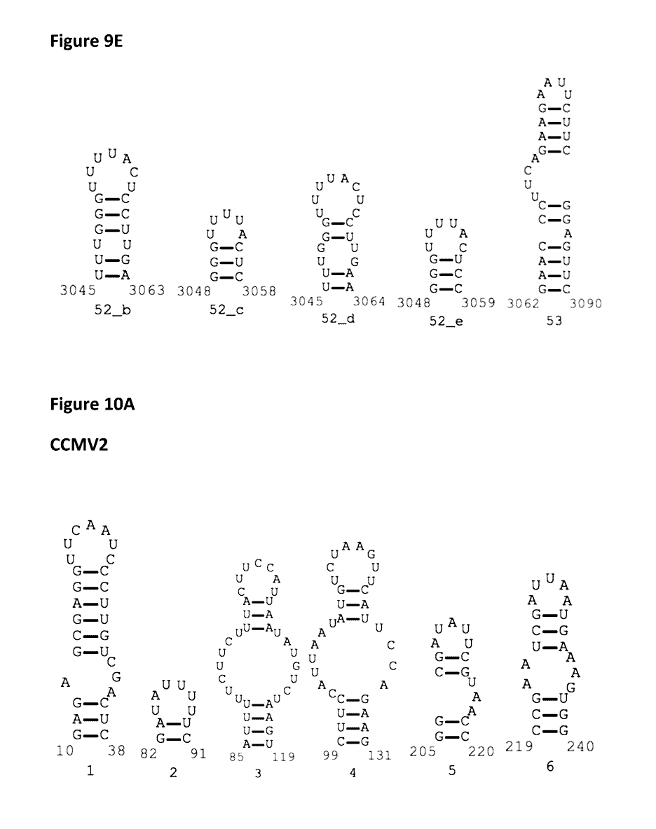

TABLE 18
Sequences for CCMV-1 PS (FIG. 9)

| SEQ | | | |
|---|---|---|---|
| ID NO: | | | Sequence |
| 296 | 10 | 38 | GAGAACGAGGUUCAAUCCCUUGUCGACUC |
| 297 | 56 | 74 | UCUUAAUUUUAUUUAAUGG |
| 298 | 88 | 95 | UCUUUUGA |
| 299 | 82 | 109 | UUUAGAUCUUUUGAAAUUGAUUUCUGAG |
| 300 | 88 | 112 | UCUUUUGAAAUUGAUUUCUGAGAGA |
| 301 | 88 | 112 | UCUUUUGAAAUUGAUUUCUGAGAGA |
| 302 | 155 | 176 | GCUGUAAAGCAAUUGCUUGAGC |
| 303 | 164 | 182 | CAAUUGCUUGAGCAAGUUG |
| 304 | 273 | 295 | UUGAUUUGAACUUAACUCAACAA |
| 305 | 302 | 324 | GCUCCCCAUAGUUUGGCUGGAGC |
| 306 | 310 | 320 | UAGUUUGGCUG |
| 307 | 349 | 360 | CUGUCUUUCAAG |
| 308 | 374 | 399 | GAUCCCAUCAUUGAUUUUGGUGGUUC |
| 309 | 428 | 455 | ACACGUAUUCACAGUUGUUGUCCCGUGU |
| 310 | 442 | 465 | UUGUUGUCCCGUGUUGGGCGUCAG |
| 311 | 445 | 465 | UUGUCCCGUGUUGGGCGUCAG |
| 312 | 593 | 610 | GCCAUAUGUAUUCAUGGU |
| 313 | 592 | 614 | GGCCAUAUGUAUUCAUGGUGGUU |
| 314 | 617 | 635 | GACAUGGGUUACACAGGUC |
| 315 | 663 | 675 | UGCGUAUUUUGCG |
| 316 | 661 | 682 | GGUGCGUAUUUUGCGGGGUACU |
| 317 | 676 | 691 | GGGUACUAUUAUGUUC |
| 318 | 674 | 692 | CGGGGUACUAUUAUGUUCG |
| 319 | 677 | 700 | GGUACUAUUAUGUUCGACGGUGCU |
| 320 | 702 | 716 | UGUUGUUUGACAACG |
| 321 | 712 | 736 | CAACGAAGGCGUUUUACCUUUGUUG |
| 322 | 719 | 743 | GGCGUUUUACCUUUGUUGAAGUGCC |
| 323 | 770 | 797 | UCUGAGGUCAUUAAAUUUGAUUUCAUGA |
| 324 | 794 | 823 | AUGAAUGAGAGCACACUUUCUUAUAUUCAU |
| 325 | 836 | 856 | CUUGGUUCAUUUUUGACUGAG |
| 326 | 978 | 996 | GUGUUUGGUUUGAGAAUAU |
| 327 | 1089 | 1111 | AAGAGAUUGCUUUUCGAUGUUUU |
| 328 | 1098 | 1117 | CUUUUCGAUGUUUUAAGGAG |
| 329 | 1144 | 1168 | AGCGAUAGCAUCUAUUCUGUCCGCU |
| 330 | 1396 | 1404 | AGAGUUUCU |
| 331 | 1400 | 1429 | UUUCUGGCUGGUAAAUUCCCUUGGCUGAGG |
| 332 | 1437 | 1465 | CGUACAAAGACAGUUUUGUUUUUCUGUCG |
| 333 | 1517 | 1533 | CUGAGGAGUUUCUUCAG |
| 334 | 1516 | 1537 | ACUGAGGAGUUUCUUCAGCAGU |
| 335 | 1554 | 1568 | GCAUUGAAUUAGAGC |
| 336 | 1557 | 1582 | UUGAAUUAGAGCUUGAAUCUGCGCAA |
| 337 | 1567 | 1578 | GCUUGAAUCUGC |
| 338 | 1622 | 1654 | AUCGAUGAGGAGGAAUUUCAAGAUGCCAUCGAU |
| 339 | 1766 | 1783 | AUCAAGGAAUUCUCUGAU |
| 340 | 1767 | 1800 | UCAAGGAAUUCUCUGAUUAUUGUCGUCGCCUUGA |
| 341 | 1790 | 1808 | CGUCGCCUUGACUGUAACG |
| 342 | 1875 | 1901 | CGAUCCUUGAAACUUAUCAUAGGGUUG |
| 343 | 1984 | 2014 | GGGCUUAGGUCCGAAGUUUGAUGAUGAGCUU |
| 344 | 2214 | 2238 | GGGAGGCUCUAUUCCCUCAUAAUCC |
| 345 | 2289 | 2309 | UGCAUGGUUUACCGCGAUGUA |
| 346 | 2312 | 2324 | CGCUUAUUGGUCG |
| 347 | 2379 | 2400 | AGUGUCAGUCUGUUCUUGCAUU |
| 348 | 2411 | 2430 | GAGCAAAUUUCUUUUAAAUC |
| 349 | 2431 | 2449 | GCGAGAUGCAACUUUCCGC |
| 350 | 2438 | 2449 | GCAACUUUCCGC |
| 351 | 2460 | 2482 | GUGAUUUGCAGUUUGACAGUCGC |
| 352 | 2512 | 2529 | GCAAGAUGUUAUUUCCGC |
| 353 | 2626 | 2649 | AGCAUCACCUUUACAGGUGACGCU |
| 354 | 2655 | 2681 | GGGAAAAAUUCUAUUUGACAAUGACUC |
| 355 | 2694 | 2717 | CCGCCCUUGUUUCCAGGGCUAAGG |
| 356 | 2696 | 2712 | GCCCUUGUUUCCAGGGC |
| 357 | 2709 | 2731 | GGGCUAAGGAUUUCCCAGAGCUU |
| 358 | 2798 | 2808 | GCUGUAUUGGU |
| 359 | 2843 | 2862 | ACUGAAGAAUAUUGCUUGGU |
| 360 | 2870 | 2894 | ACUCGACAUAAGAUUACCUUUGAGU |
| 361 | 2875 | 2904 | ACAUAAGAUUACCUUUGAGUAUCUUUAUGU |
| 362 | 2893 | 2910 | GUAUCUUUAUGUUGGUAU |
| 363 | 2892 | 2913 | AGUAUCUUUAUGUUGGUAUGCU |
| 364 | 2953 | 2979 | AGUGUGAUUCACUUACGAAUCAGUUCU |
| 365 | 3042 | 3051 | GCCUUUGGGU |
| 366 | 3045 | 3063 | UUUGGGUUUUACUCCUUGA |
| 367 | 3048 | 3058 | GGGUUUUACUC |
| 368 | 3048 | 3059 | GGGUUUUACUCC |
| 369 | 3045 | 3064 | UUUGGGUUUUACUCCUUGAA |
| 370 | 3062 | 3090 | GAACCCUUCAGAAGAAUUCUUCGGAGUUC |

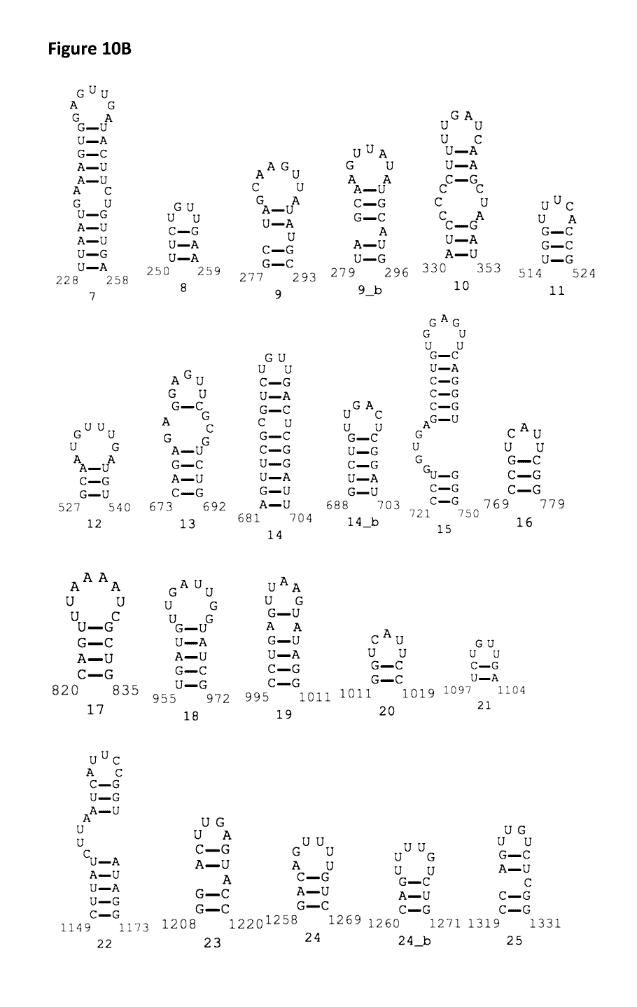

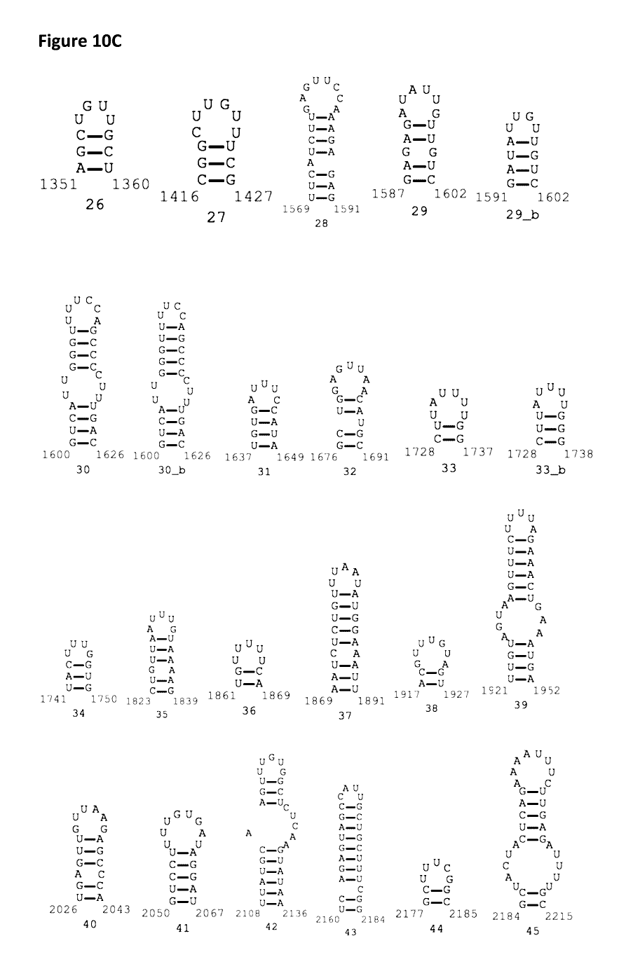

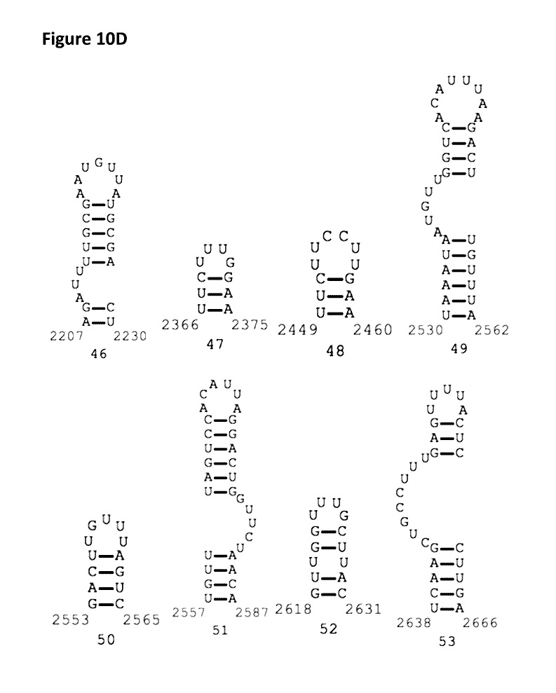

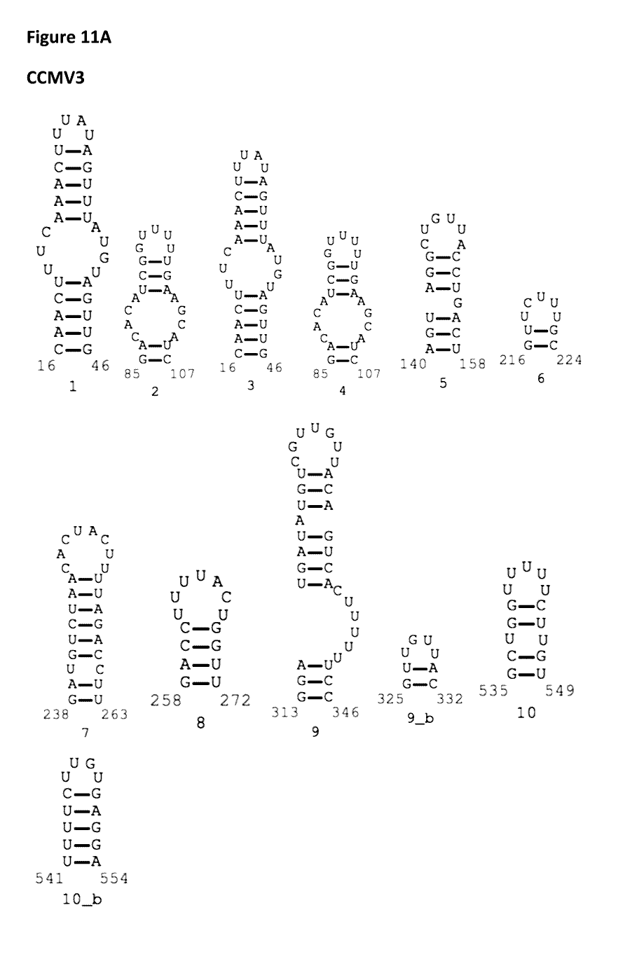

TABLE 19
Sequences for CCMV-2 PS (FIG. 10)

| SEQ | | | |
|---|---|---|---|
| ID | | | |
| NO: | | | Sequence |
| 371 | 10 | 38 | GAGAGCGAGGUUCAAUCCCUUGUCGACUC |
| 372 | 82 | 91 | GAUAUUUUUC |
| 373 | 85 | 119 | AUUUUUCUUCUUUACUUCCAUUAAUAUGUCUAAGU |
| 374 | 99 | 131 | CUUCCAUUAAUAUGUCUAAGUUCAUUCCAGAAG |
| 375 | 205 | 220 | GGCGAUAUUCGUAACC |
| 376 | 219 | 240 | CCGAAUCGAUUAAUGAAAGUGG |
| 377 | 228 | 258 | UUAAUGAAAGUGGAGUUGAUACUUCUGUUGA |
| 378 | 250 | 259 | UUCUGUUGAA |
| 379 | 277 | 293 | GCUAGCAAGUUAUAUGC |
| 380 | 279 | 296 | UAGCAAGUUAUAUGCAUG |
| 381 | 330 | 353 | AUCCCCCUUUUGAUCAAGCUAGAU |
| 382 | 514 | 524 | UGGUUUCACCG |
| 383 | 527 | 540 | GCAAUGUUUGAUGU |
| 384 | 673 | 692 | CAGAGAGGAGUUCGCGUCUG |
| 385 | 681 | 704 | AGUUCGCGUCUGUUGACUCGGAUU |
| 386 | 688 | 703 | GUCUGUUGACUCGGAU |
| 387 | 721 | 750 | CCUGGUGAGCCCUGUGGAGUUCAGGGUGGG |
| 388 | 769 | 779 | CCGUCAUUCGG |
| 389 | 820 | 835 | CAGUUUAAAAUCGCUG |
| 390 | 955 | 972 | UGAUGUUGAUUGGUAUCG |
| 391 | 995 | 1011 | CCUGAGUUAAGUAUAGG |
| 392 | 1011 | 1019 | GGUCAUUCC |
| 393 | 1097 | 1104 | UCUGUUGA |
| 394 | 1149 | 1173 | CUUAUCUUAAUCAUUCCGGUAUAGG |
| 395 | 1208 | 1220 | GGACUUGAGUACC |
| 396 | 1258 | 1269 | GACAGUUUUGUC |
| 397 | 1260 | 1271 | CAGUUUUGUCUG |
| 398 | 1319 | 1331 | CCAGUUGUCUCGG |
| 399 | 1351 | 1360 | AGCUGUUGCU |
| 400 | 1416 | 1427 | CGGCUUGUUUCG |
| 401 | 1569 | 1591 | UUCAUCUUGAGUUCCAAAGAGAG |
| 402 | 1587 | 1602 | GAGAGAUAUUGUUGUC |
| 403 | 1591 | 1602 | GAUAUUGUUGUC |
| 404 | 1600 | 1626 | GUCAUUGGGUUUUCCAGCCCCUUUGAC |
| 405 | 1600 | 1626 | GUCAUUGGGUUUUCCAGCCCCUUUGAC |
| 406 | 1637 | 1649 | UGUGAUUUCCAUA |
| 407 | 1676 | 1691 | GCUGGAGUUAACAUGC |
| 408 | 1728 | 1737 | CUUAUUUUGG |
| 409 | 1728 | 1738 | CUUAUUUUGGG |
| 410 | 1741 | 1750 | UACUUUGGUG |
| 411 | 1823 | 1839 | CUGUUAAUUUGUAAAAG |
| 412 | 1861 | 1869 | UGUUUUUCA |
| 413 | 1869 | 1891 | AAUCUCUGUUUAAUAUGGAAAUU |
| 414 | 1917 | 1927 | ACGUUUGUAGU |
| 415 | 1921 | 1952 | UUGUAGUAAGUUUCUUUUAGAAACUGAAAUGA |
| 416 | 2026 | 2043 | UGAGUUGUUAAGAGCCCA |
| 417 | 2050 | 2067 | GUCCUUUUGUGAUAGGAU |
| 418 | 2108 | 2136 | UUAUGCAAGUUUGUGGCUCUCAAGUAUAA |
| 419 | 2160 | 2184 | UCAGAGUAGCCAUUGCUGCUUUCGG |
| 420 | 2177 | 2185 | GCUUUCGGC |
| 421 | 2184 | 2215 | GCUACUACUCAGAAAAUUUCUUGAGAUUUUGC |
| 422 | 2207 | 2230 | AGAUUUUGCGAAUGUUAUGCGACU |
| 423 | 2366 | 2375 | UUCUUUGGAA |
| 424 | 2449 | 2460 | UUCUUCCUUGAA |
| 425 | 2530 | 2562 | UAAAUAAUGUUGGUCACAUUUAAGACUUGUUUA |
| 426 | 2553 | 2565 | GACUUGUUUAGUC |
| 427 | 2557 | 2587 | UGUUUAGUCCACAUUAGGACUGGUUCUAACA |
| 428 | 2618 | 2631 | GUUGGUUUGCUUAC |
| 429 | 2638 | 2666 | UCAAGCUGCCUUUGAGUUUUACUCCUUGA |

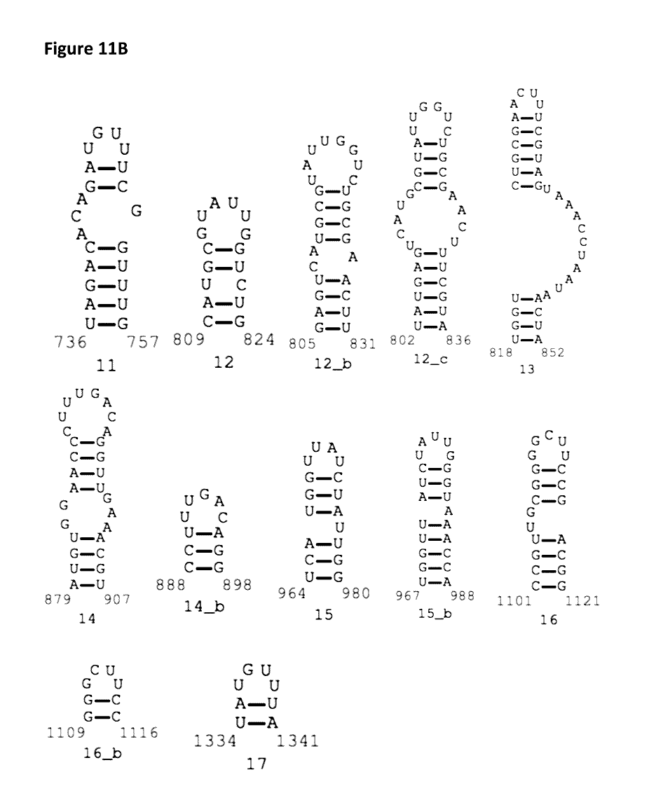

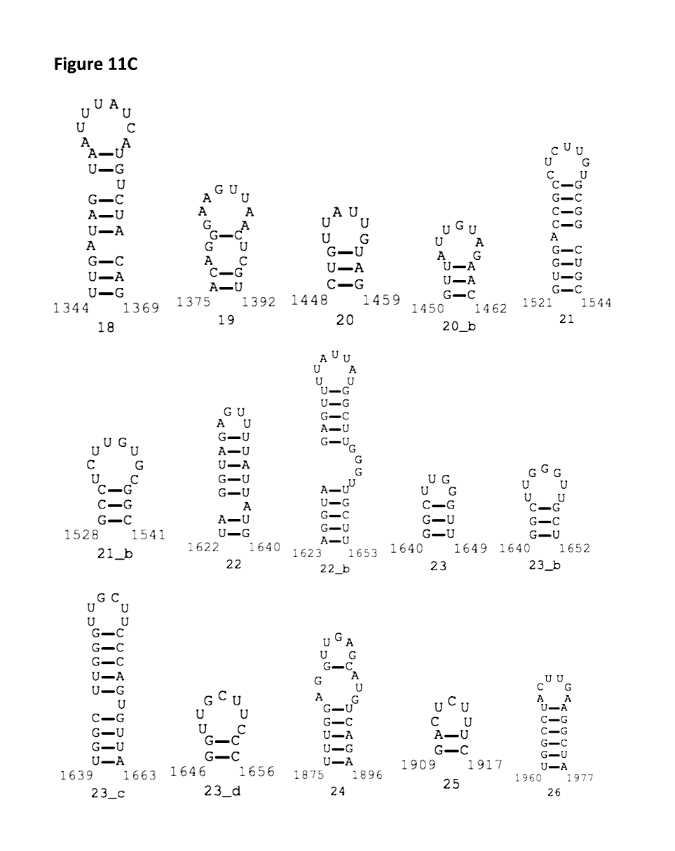

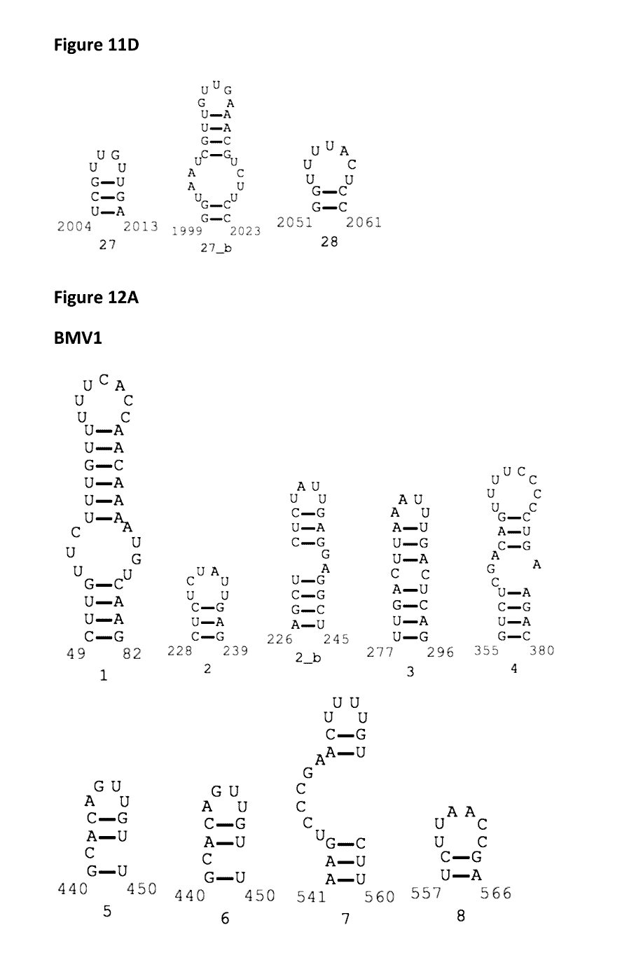

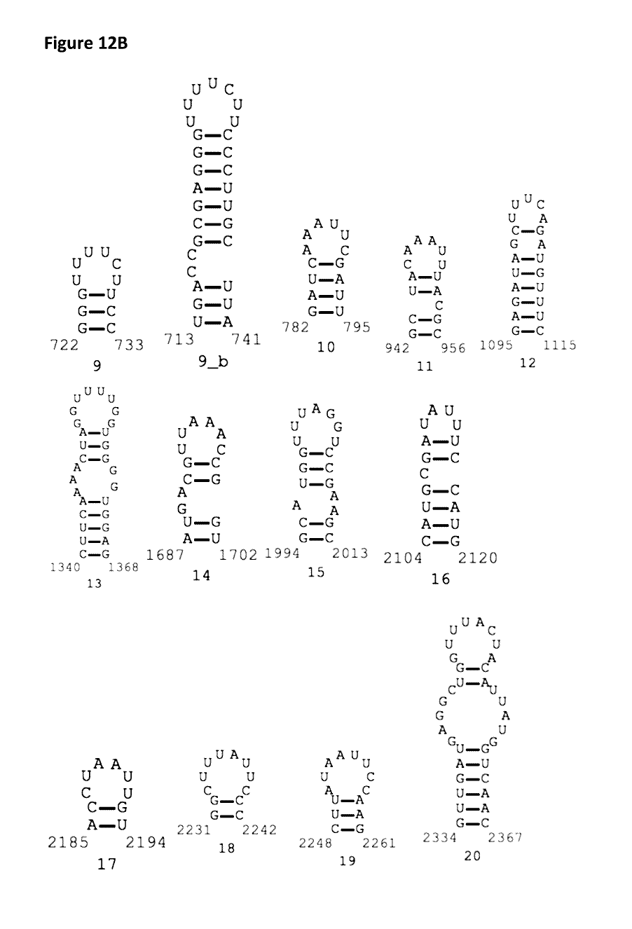

TABLE 20
Sequences for CCMV-3 PS (FIG. 11)

| SEQ | | | |
|---|---|---|---|
| ID | | | |
| NO: | | | Sequence |
| 430 | 16 | 46 | CAACUUUCAAACUUUAUAGUUUAUGUAGUUG |
| 431 | 85 | 107 | GACACAUCGGUUUUUGAAGCAUC |
| 432 | 16 | 46 | CAACUUUCAAACUUUAUAGUUUAUGUAGUUG |
| 433 | 85 | 107 | GACACAUCGGUUUUUGAAGCAUC |
| 434 | 140 | 158 | AGUAGGCUGUUACCUGACU |
| 435 | 216 | 224 | GUUCUUUGC |
| 436 | 238 | 263 | GAUGUCUAACACUACUUUUAGACCUU |
| 437 | 258 | 272 | GACCUUUUACUGGUU |
| 438 | 313 | 346 | GGAUGAUAUGUCGUUGUUACAGUCACUUUUUUCC |
| 439 | 325 | 332 | GUUGUUAC |
| 440 | 535 | 549 | GCUGGUUUUUCUUGU |
| 441 | 541 | 554 | UUUUCUUGUGAGGA |
| 442 | 736 | 757 | UAGACACAGAUGUUUCGGUUUG |
| 443 | 809 | 824 | CAUGCGUAUUGGUCUG |
| 444 | 805 | 831 | GAGUCAUGCGUAUUGGUCUGCGAACUU |
| 445 | 802 | 836 | UAUGAGUCAUGCGUAUUGGUCUGCGAACUUUCGUA |
| 446 | 818 | 852 | UGGUCUGCGAACUUUCGUAGUAAACCUAAUAACUA |
| 447 | 879 | 907 | AUGUGGAACCCUUUGACAGGUUGAAACGU |
| 448 | 888 | 898 | CCUUUGACAGG |
| 449 | 964 | 980 | UCAUGGUUAUCUAUUGG |
| 450 | 967 | 988 | UGGUUAUCUAUUGGGUAAACCA |
| 451 | 1101 | 1121 | CCGUUGCGGGGCUUCCGACGG |
| 452 | 1109 | 1116 | GGGCUUCC |
| 453 | 1334 | 1341 | UAUGUUUA |
| 454 | 1344 | 1369 | UUGAUAGUAAUUUAUCAUGUCUACAG |
| 455 | 1375 | 1392 | ACAGGGAAGUUAACUCGU |
| 456 | 1448 | 1459 | CUGUUAUUGUAG |
| 457 | 1450 | 1462 | GUUAUUGUAGAAC |
| 458 | 1521 | 1544 | GUGGACCGCCUCUUGUGCGGCUGC |
| 459 | 1528 | 1541 | GCCUCUUGUGCGGC |
| 460 | 1622 | 1640 | UAGGUAGAGUUUUAUUAUG |
| 461 | 1623 | 1653 | AGGUAGAGUUUUAUUAUGGCUUGGGUUGCUU |
| 462 | 1640 | 1649 | GGCUUGGGUU |
| 463 | 1640 | 1652 | GGCUUGGGUUGCU |
| 464 | 1646 | 1656 | GGUUGCUUCCC |
| 465 | 1639 | 1663 | UGGCUUGGGUUGCUUCCCAGUGUUA |
| 466 | 1875 | 1896 | UUUGGAGGUUGAGCAUGUCAGA |
| 467 | 1909 | 1917 | GACUCUUUC |
| 468 | 1960 | 1977 | UGGCCUACUUGAAGGCUA |
| 469 | 2004 | 2013 | UCGUUGUUGA |
| 470 | 1999 | 2023 | GGUAAUCGUUGUUGAAACGUCUUCC |
| 471 | 2051 | 2061 | GGUUUUACUCC |

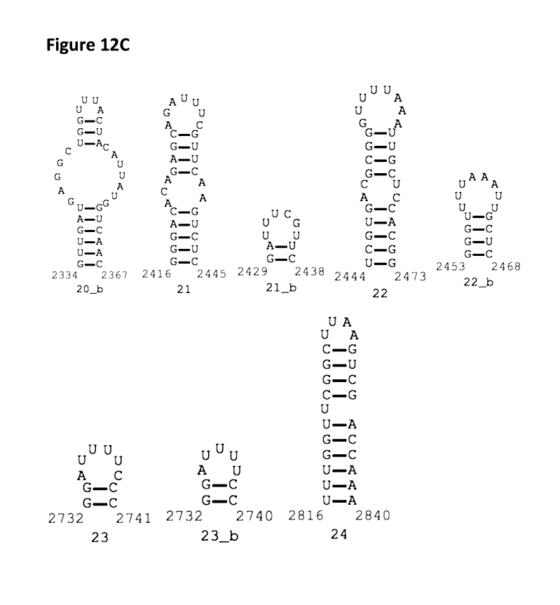

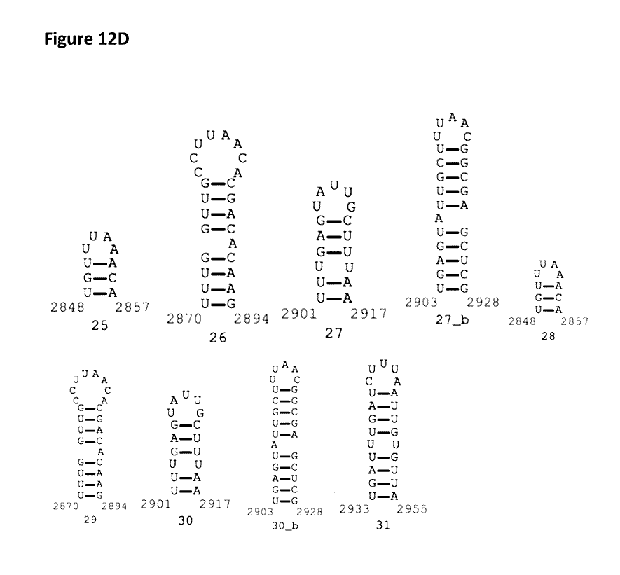

TABLE 21
Sequences for BMV-1 PS (FIGS. 12A-12D)

| SEQ ID No. | Sequence |
|---|---|
| 145 | CUUGUUCUUU GUUUUUCACC AACAAAAUGU CAAG |
| 146 | CUCUCUAUUG AG |
| 147 | AGCUCUCUAU UGAGGAGGCU |
| 148 | UUGACUUAAA UUUGACUCAG |
| 149 | GUCUCGACAG UUUUCCCCCU GAAGAC |
| 150 | GCACAGUUGU U |
| 151 | GCACAGUUGU U |
| 152 | AAGUCCCGAA CUUUUGUCUU |
| 153 | UCUUAACCGA |
| 154 | UGACCGCGAG GGUUUUCUUC CCUUGCUUA |
| 155 | GGGUUUUCUU CC |
| 156 | GAUCAAAUUC GAUU |
| 157 | GCUACAAAUU UACGC |
| 158 | GAGAUAGCUU UCAGAUGUUU C |
| 159 | CUUCAAAACU AGGUUUUGGU GGGGUGGAG |
| 160 | AUGACGUUAA ACCGGU |
| 161 | GCAUGGUUUA GGUCCGAAGC |
| 162 | CAUGCGAUAU UUCCAUG |
| 163 | ACCUAAUUGU |
| 164 | GGCUUUAUUC CC |
| 165 | CUUAUAAUUC CAAG |
| 166 | GUUGAUGAGG CUGGUUUACU ACAUUAUGGU CAAC |
| 167 | GUUGAUGAGG CUGGUUUACU ACAUUAUGGU CAAC |
| 168 | GGGACACAGA GCAGAUUUCG UUCAAGUCUC |
| 169 | GAUUUCGUUC |
| 170 | UCGUGACGCG GGUUUUAAAU UGCUCCACGG |
| 171 | GGGUUUUAAA UUGCUC |
| 172 | GGAUUUUCCC |
| 173 | GGAUUUUCC |
| 174 | UUUGGUUCGG CUUAAGUCGA CCAAA |
| 175 | UGUUUAAACA |
| 176 | UUUGGUUGCC UUAACACGAC ACAAG |
| 177 | UUUGAGUAUU GCUUUAA |
| 178 | UGAGUAUUGC UUUAACGGCG AGCUCG |
| 179 | UGUUUAAACA |
| 180 | UUUGGUUGCC UUAACACGAC ACAAG |
| 181 | UUUGAGUAUU GCUUUAA |
| 182 | UGAGUAUUGC UUUAACGGCG AGCUCG |
| 183 | UGAUUUGAUC UUUAAUUGUG UUA |

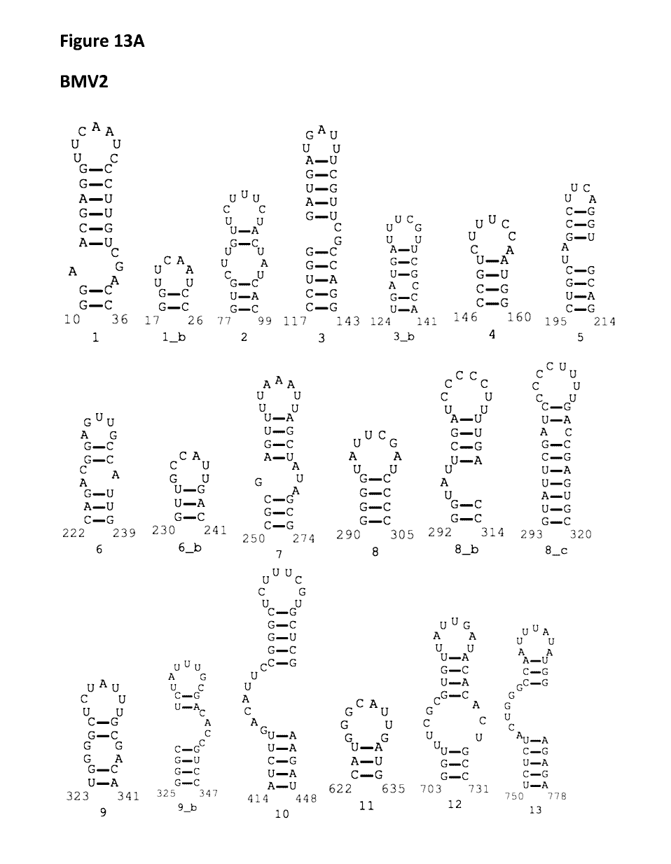

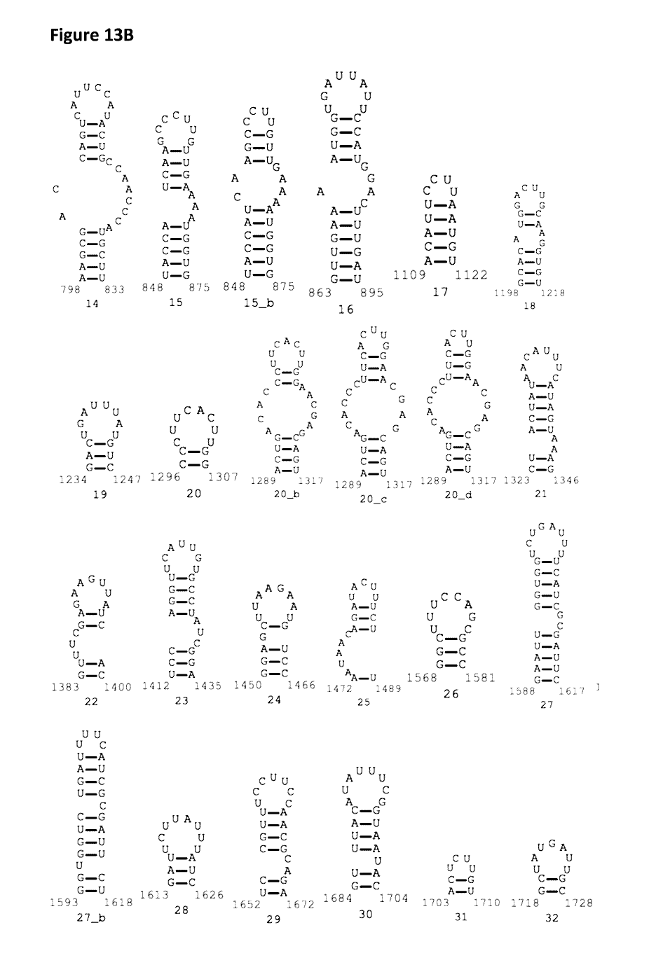

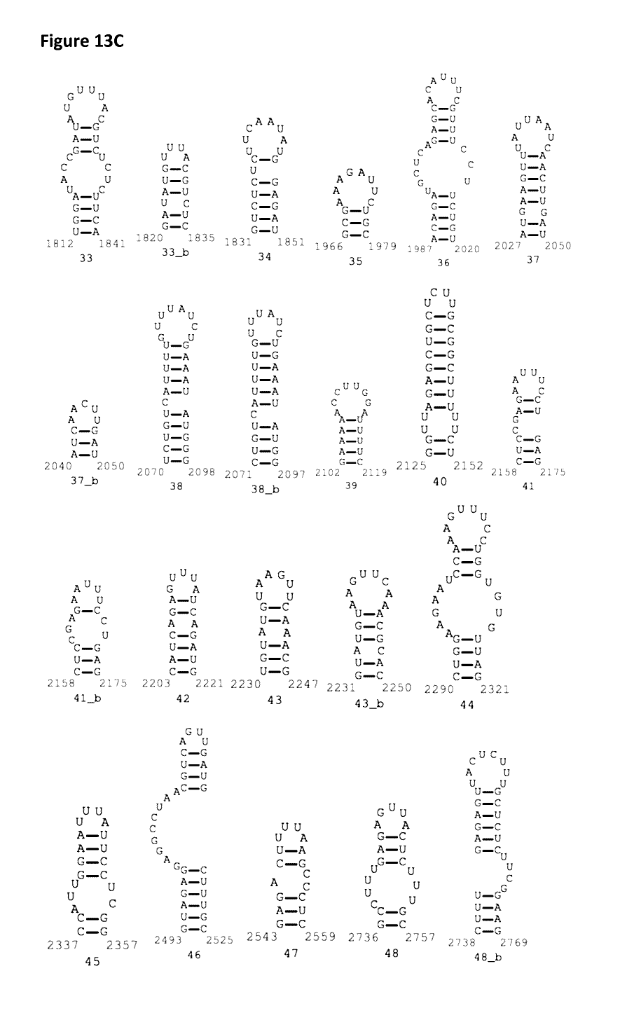

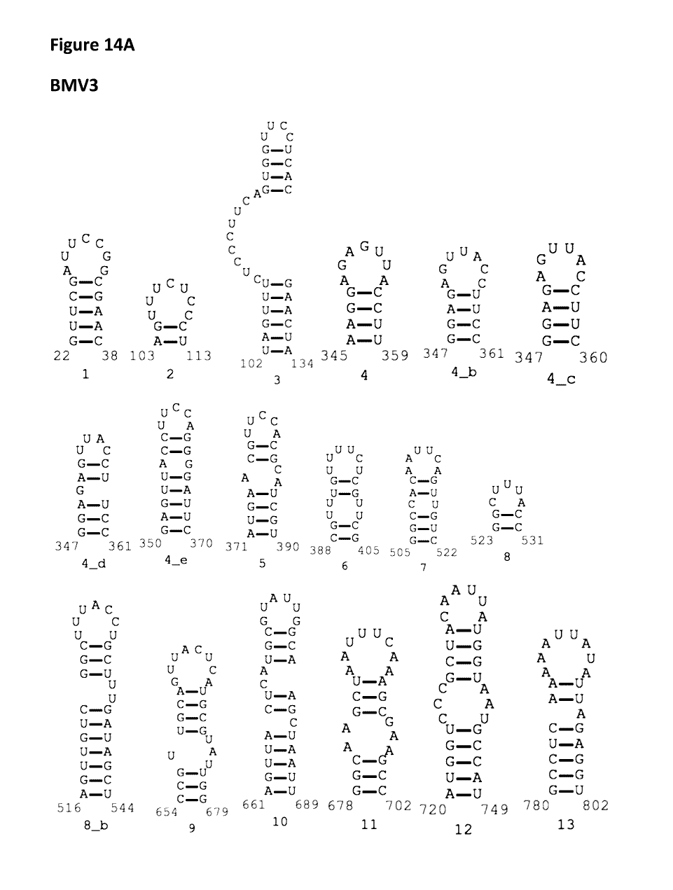

TABLE 22
Sequences for BMV-2 PS (FIGS. 13A-13C)

| SEQ | | | |
|---|---|---|---|
| ID | | | |
| NO: | | | Sequence |
| 192 | 10 | 36 | GGAACGAGGUUCAAUCCCUUGUCGACC |
| 193 | 17 | 26 | GGUUCAAUCC |
| 194 | 77 | 99 | GUGCUUGUUCUUUCUACUAUCAC |
| 195 | 117 | 143 | CCUGGGAUGAUGAUUUCGUUCGCCAGG |
| 196 | 124 | 141 | UGAUGAUUUCGUUCGCCA |
| 197 | 146 | 160 | CCGUCUUUCCAAUGG |
| 198 | 195 | 214 | CUGCUAGCCUUCAGGUGCAG |
| 199 | 222 | 239 | CAGACGGAGUUGCCAUUG |
| 200 | 230 | 241 | GUUGCCAUUGAC |
| 201 | 250 | 274 | CGCGAGUUUUAAAUUAGCUAUAGCG |
| 202 | 290 | 305 | GGGGUAUUCGAUCCCC |
| 203 | 292 | 314 | GGUAUUCGAUCCCCCUUUUGACC |
| 204 | 293 | 320 | GUAUUCGAUCCCCCUUUUGACCGAGUGC |
| 205 | 323 | 341 | UGGGGCUCUAUUUGCGACA |
| 206 | 325 | 347 | GGGCUCUAUUUGCGACACCGUCC |
| 207 | 414 | 448 | AUCUUGACAUUCCGGGCUCUUUCGUGCUCGAAGAU |
| 208 | 622 | 635 | CAUGGGCAUUGAUG |
| 209 | 703 | 731 | GGUUUCGCGUGUUAUUGAUACACACUGCC |
| 210 | 750 | 778 | UCUCUACUGGGCCAAUUUAUAUGGAGAGA |
| 211 | 798 | 833 | AAGCGACCAGUCAUUCCAUACUGCCAACCCAUGCUU |
| 212 | 848 | 875 | UACCAUCAAGCCCUUGUUGAAAAUGGUG |
| 213 | 848 | 875 | UACCAUCAAGCCCUUGUUGAAAAUGGUG |
| 214 | 863 | 895 | GUUGAAAAUGGUGAUUAUUCCAUGGACUUUGAU |
| 215 | 1109 | 1122 | ACAUUCCUUAAUGU |
| 216 | 1198 | 1218 | GCACAUGGACUUGCAAGGUGU |
| 217 | 1234 | 1247 | GACUGAUUUAUGUC |
| 218 | 1296 | 1307 | CCCUUCACUUGG |
| 219 | 1289 | 1317 | ACUGACACCCUUCACUUGGAACGAGCAGU |
| 220 | 1289 | 1317 | ACUGACACCCUUCACUUGGAACGAGCAGU |
| 221 | 1289 | 1317 | ACUGACACCCUUCACUUGGAACGAGCAGU |
| 222 | 1323 | 1346 | CUACUAUAACAUUUCAUAGUAAAG |
| 223 | 1383 | 1400 | GUUUCGAGAAGUUAUCAC |
| 224 | 1412 | 1435 | UCCAGGUUCAUUGUGCCUAUCGGA |
| 225 | 1450 | 1466 | GGAGCUUAAGAAUGUCC |
| 226 | 1472 | 1489 | AAUAACAGAUACUUUCUU |
| 227 | 1568 | 1581 | GGCUUUCCAGCGCC |
| 228 | 1588 | 1617 | GAAUUGGUGGUCUGAUUUUCAUCGCGAUUC |
| 229 | 1593 | 1618 | GGUGGUCUGAUUUUCAUCGCGAUUCU |
| 230 | 1613 | 1626 | GAUUCUUAUUUAUC |
| 231 | 1652 | 1672 | UCCGUUUCCUUCCAACGCAGA |
| 232 | 1684 | 1704 | GUUUACAUAUUUCGGUAAUAC |
| 233 | 1703 | 1710 | ACUCUUGU |
| 234 | 1718 | 1728 | GCUAUGAUUGC |
| 235 | 1812 | 1841 | UGGAUACCGAUAUGUUUACGUCUCUCUUCA |
| 236 | 1820 | 1835 | GAUAUGUUUACGUCUC |
| 237 | 1831 | 1851 | GUCUCUCUUCAAUAUGGAGAU |
| 238 | 1966 | 1979 | GCGAAAGAUUCUGC |
| 239 | 1987 | 2020 | ACAGAUGCUCAGAGCACAUUUCGUUUCCUUCUGU |
| 240 | 2027 | 2050 | AUGAAGUUUAUUAAUCAACUUGAU |
| 241 | 2040 | 2050 | AUCAACUUGAU |
| 242 | 2070 | 2098 | UCUGUCAUUUUGUUUAUCUGAAAUAUGGG |
| 243 | 2071 | 2097 | CUGUCAUUUUGUUUAUCUGAAAUAUGG |
| 244 | 2102 | 2119 | GAAAAACCUUGGAUUUUC |
| 245 | 2125 | 2152 | GGUUAGAGCUGCUCUUGCGGCUUUUUCU |
| 246 | 2158 | 2175 | CUCCGAGAAUUUCCUGAG |
| 247 | 2158 | 2175 | CUCCGAGAAUUUCCUGAG |
| 248 | 2203 | 2221 | CAUCAGAGUUUAUCAGAUG |
| 249 | 2230 | 2247 | UGUAUGUAAGUUCAAACG |
| 250 | 2231 | 2250 | GUAUGUAAGUUCAAACGCAC |
| 251 | 2290 | 2321 | CUGGAAGAAUCCAAAGUUUCCUGGUGUGUUAG |
| 252 | 2337 | 2357 | CCAUUGGAAUUUAUUCCUCGG |
| 253 | 2493 | 2525 | GUAGAGGAGGCCUAACGUCAGUUGAUGCUUUGC |
| 254 | 2543 | 2559 | GAGACUUUUAAGCCCUC |
| 255 | 2736 | 2757 | GCCUUUGAGAGUUACUCUUUGC |
| 256 | 2738 | 2769 | CUUUGAGAGUUACUCUUUGCUCUCUUCGGAAG |

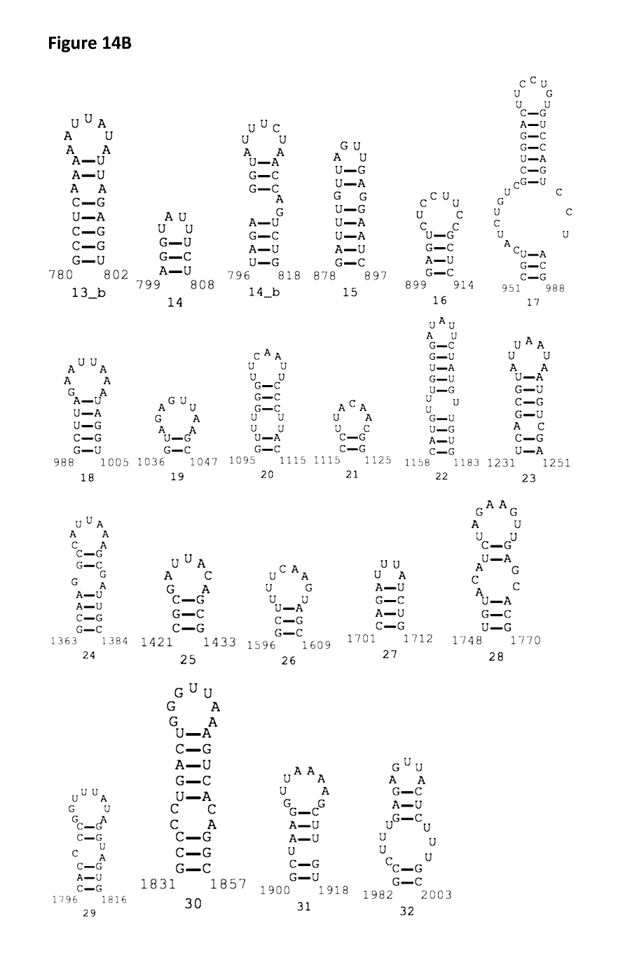

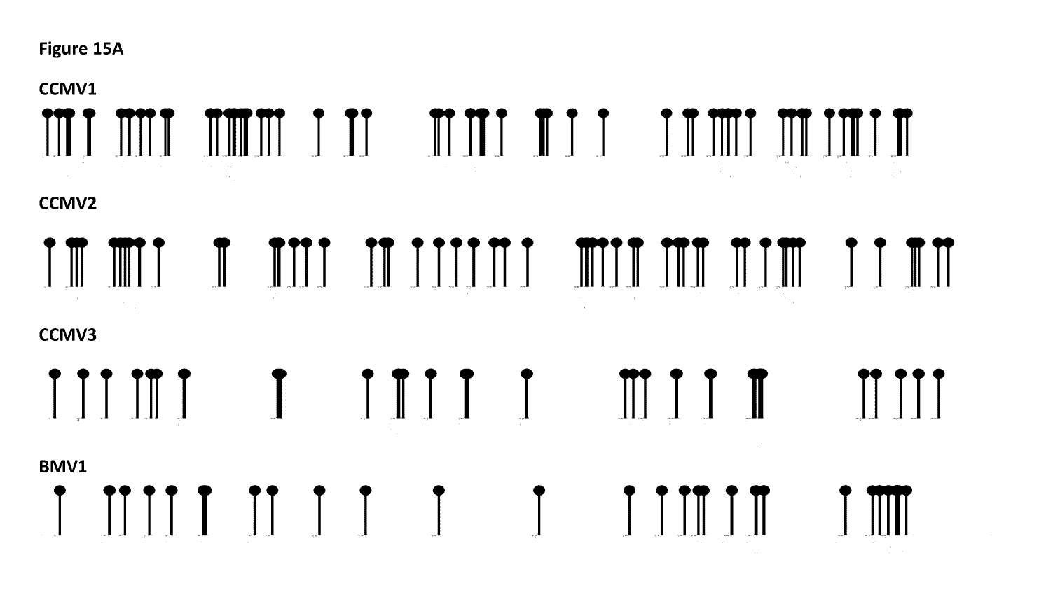

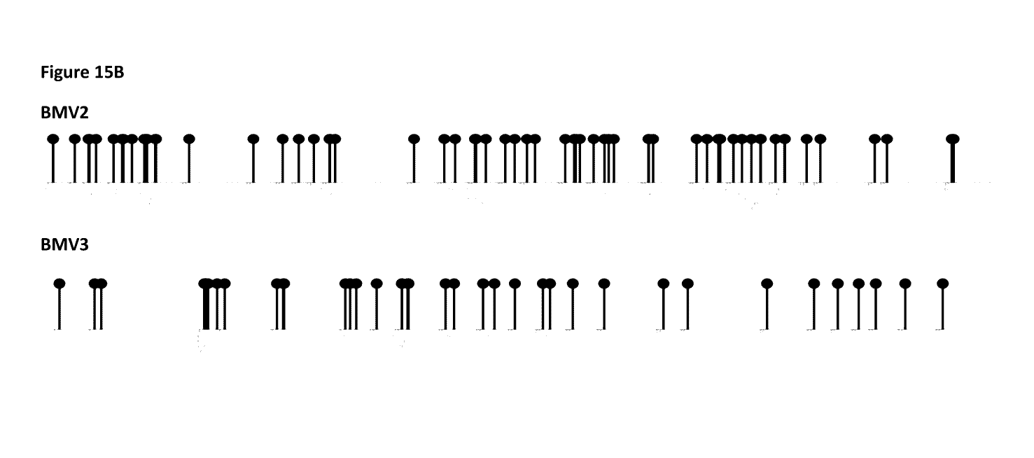

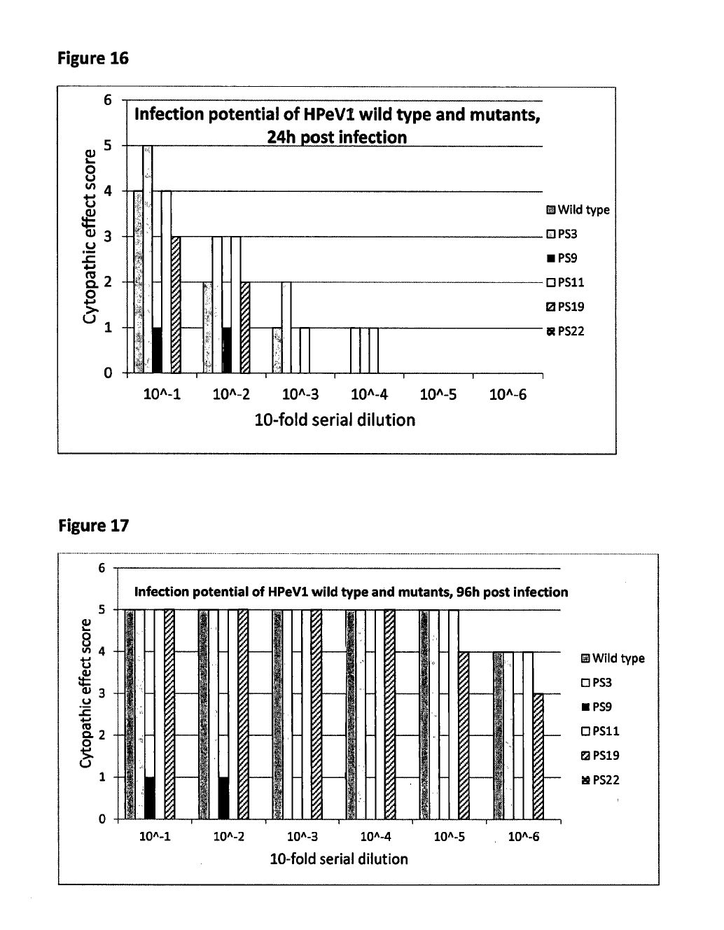

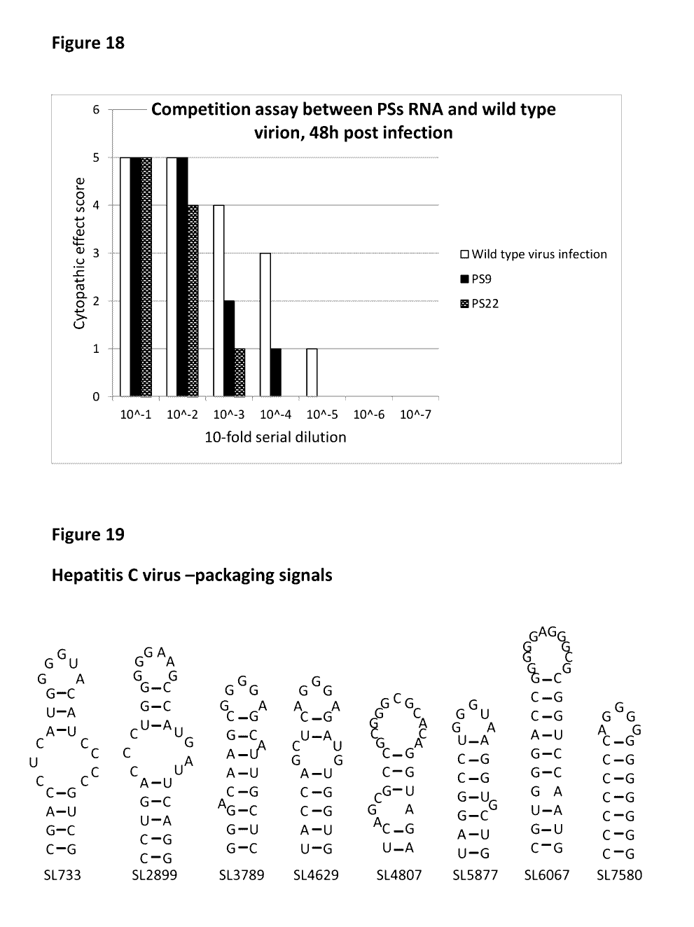

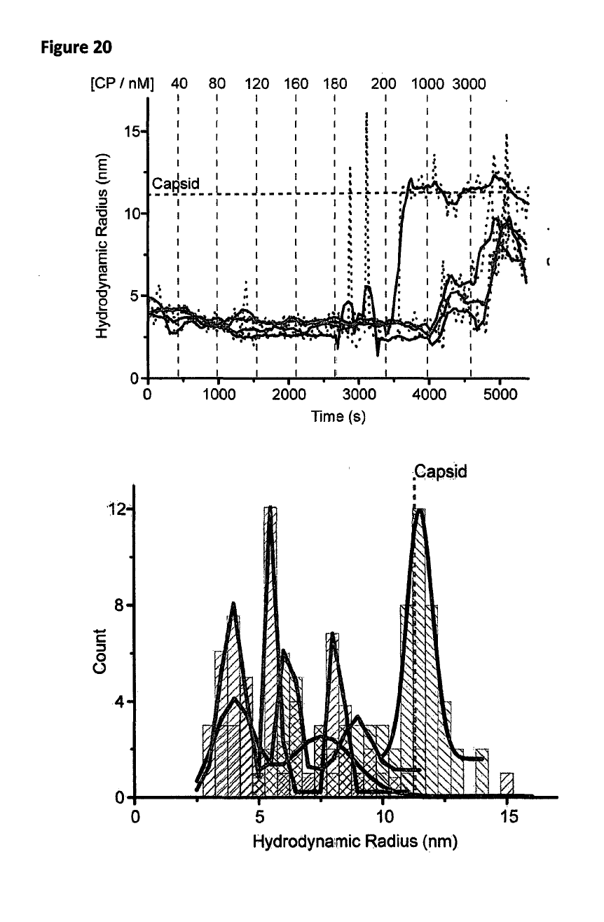

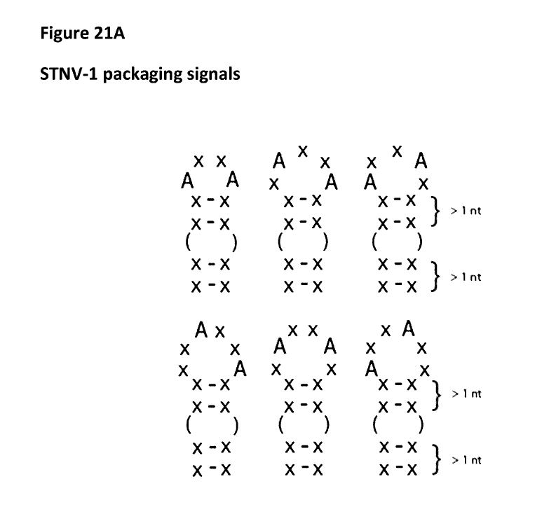

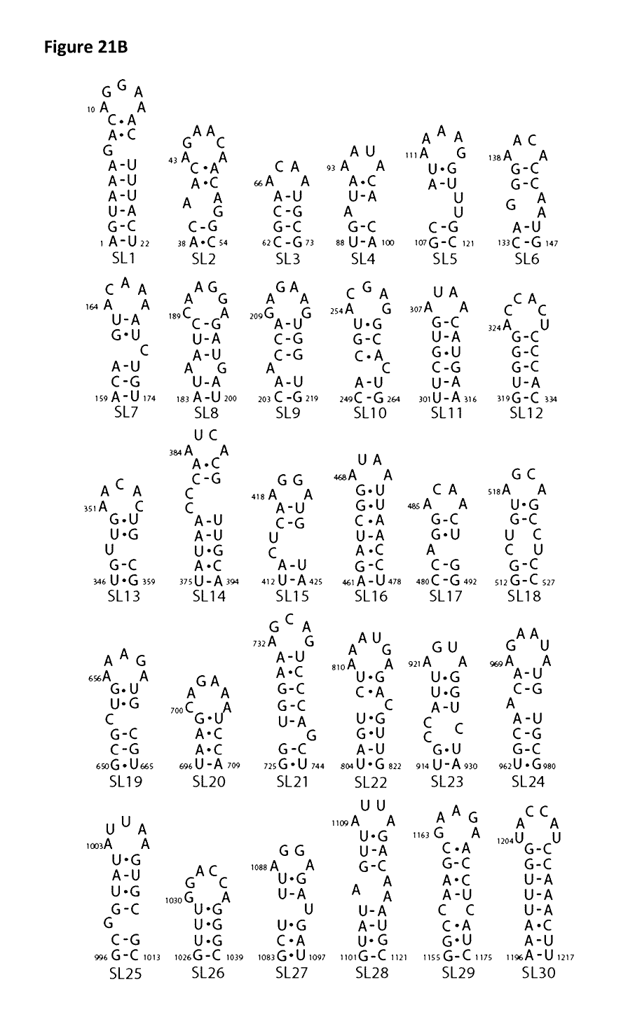

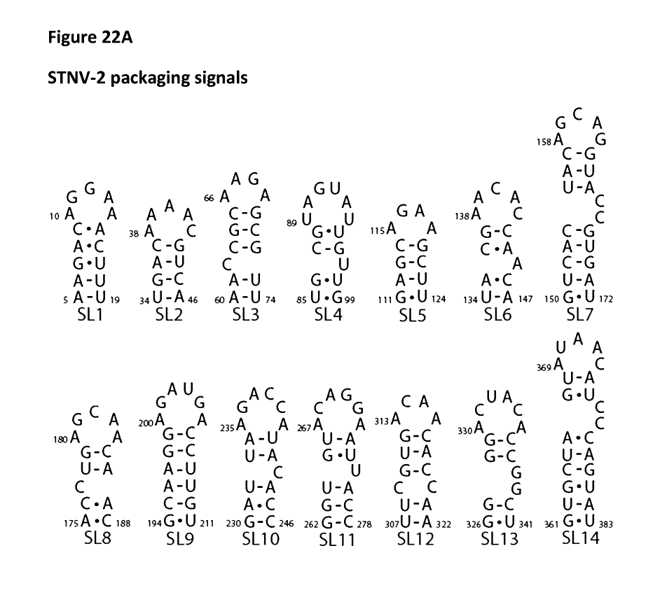

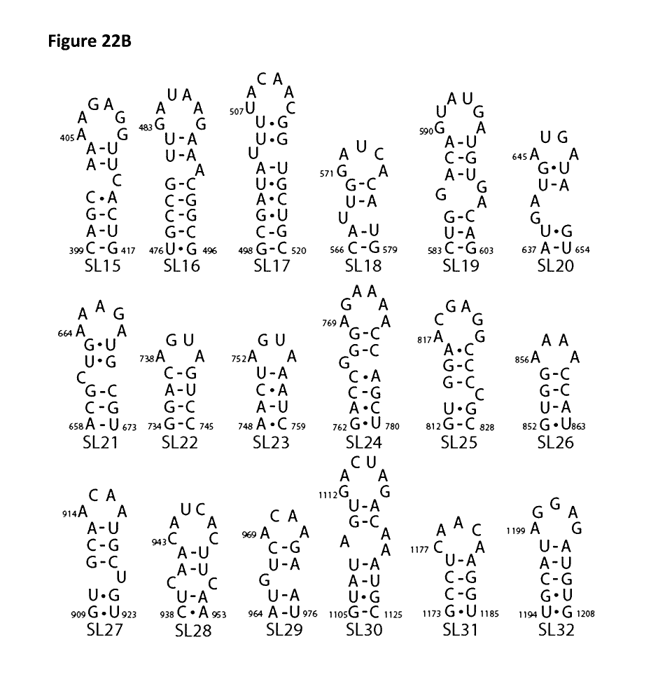

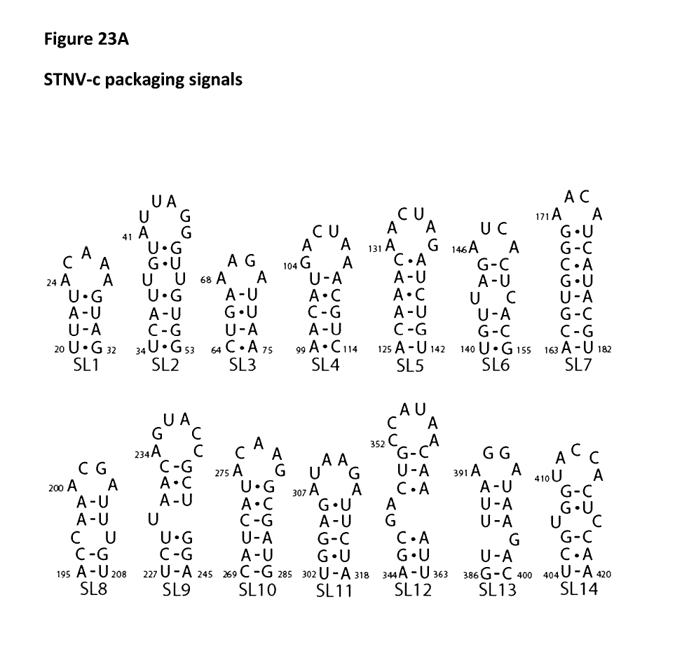

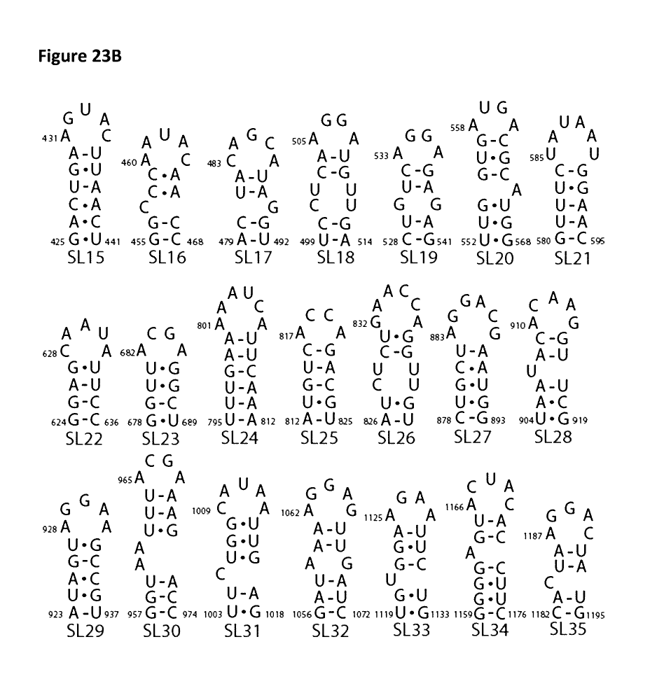

TABLE 23
Sequences for BMV-3 PS (FIGS. 14A-14B)

| SEQ | | | |
|---|---|---|---|
| ID | | | |
| NO: | | | Sequence |
| 257 | 22 | 38 | GUUCGAUUCCGGCGAAC |
| 258 | 103 | 113 | AGUUUCUCCCU |
| 259 | 102 | 134 | UAGUUUCUCCCUUCAGUGGUUCCUCACGAACUA |
| 260 | 345 | 359 | AAGGAGAGUUACCUU |
| 261 | 347 | 360 | GGAGAGUUACCUUC |
| 262 | 347 | 361 | GGAGAGUUACCUUCC |
| 263 | 347 | 361 | GGAGAGUUACCUUCC |
| 264 | 350 | 370 | GAGUUACCUUCCAGGGGAUUC |
| 265 | 371 | 390 | AUGAACGUUCCACGCAUCGU |
| 266 | 388 | 405 | CGUUUGUUUUCUCGUUCG |
| 267 | 505 | 522 | GGCCACAAUUCAGUUGUC |
| 268 | 523 | 531 | GGCUUUACC |
| 269 | 516 | 544 | AGUUGUCGGCUUUACCUGCUUUGAUAGCU |
| 270 | 654 | 679 | CCGUUGCAGUUACUCAUGCGUAUUGG |
| 271 | 661 | 689 | AGUUACUCAUGCGUAUUGGCAAGCUAAUU |
| 272 | 678 | 702 | GGCAAGCUAAUUUCAAAGCGAAGCC |
| 273 | 720 | 749 | AUGGUCCCGCUACAAUUAUGGUAAUGCCAU |
| 274 | 780 | 802 | GCCUCAAAAAUUAUAUUAGAGGU |
| 275 | 780 | 802 | GCCUCAAAAAUUAUAUUAGAGGU |
| 276 | 799 | 808 | AGGUAUUUCU |
| 277 | 796 | 818 | UAGAGGUAUUUCUAACCAGUCUG |
| 278 | 878 | 897 | GAUUUGUUAGUUGAGGAAUC |
| 279 | 899 | 914 | GAGUCUCCUUCCGCUC |
| 280 | 951 | 988 | CGUCAUCUGUCGCUGGACUUCCUGUGUCCA |
| | | | GUCCUACG |
| 281 | 988 | 1005 | GCUUAGAAUUAAAUAGGU |
| 282 | 1036 | 1047 | GUAGAGUUAAGC |
| 283 | 1095 | 1115 | GUUUGGGUUCAAUUCCCUUAC |
| 284 | 1115 | 1125 | CCUUACAACGG |
| 285 | 1158 | 1183 | CAUGUUUGUGGAUAUUCUAUGUUGUG |
| 286 | 1231 | 1251 | UCAGCGUAUUAAUAAUGUCGA |
| 287 | 1363 | 1384 | GCAAGGCCAUUAAAGCGAUUGC |
| 288 | 1421 | 1433 | CGCGAUUACAGCG |
| 289 | 1596 | 1609 | GCUUUUCAAGUAGC |
| 290 | 1701 | 1712 | CAGAUUUAUCUG |
| 291 | 1748 | 1770 | UGUACAUCUAGAAGUUGAGCACG |
| 292 | 1796 | 1816 | CACCCCGGUUUAUAGGUAGUG |
| 293 | 1831 | 1857 | GCCCCUGACUGGGUUAAAGUCACAGGC |
| 294 | 1900 | 1918 | GCUAAGGUUAAAAGCUUGU |
| 295 | 1982 | 2003 | GCCUUUGAGAGUUACUCUUUGC |

TABLE 24
Sequences for HCV PS (FIG. 19)

| SEQ ID | | | |
|---|---|---|---|
| NO: | | | Sequence |
| 184 | SL733 | 733 | CGACCTCATGGGGTACATCCCCGTCG |
| 185 | SL2899 | 2899 | CCTGACCCTGGGGGAAGCCATGATTCAGG |
| 186 | SL3789 | 3789 | GGGACAAGCGGGGAGCATTGCTC |
| 187 | SL4629 | 4629 | TACCAGCTCAGGGAGATGTGGTG |
| 188 | SL4807 | 4807 | TCAGCGCCGCGGGCGCACAGGTAG |
| 189 | SL5877 | 5877 | TAGGCCTGGGTAAGGTGCTG |
| 190 | SL6067 | 6067 | CGTGGGACCGGGGGAGGGCGCGGTCCAATG |
| 191 | SL7580 | 7580 | CCCCCCCAGGGGGGGGGGG |

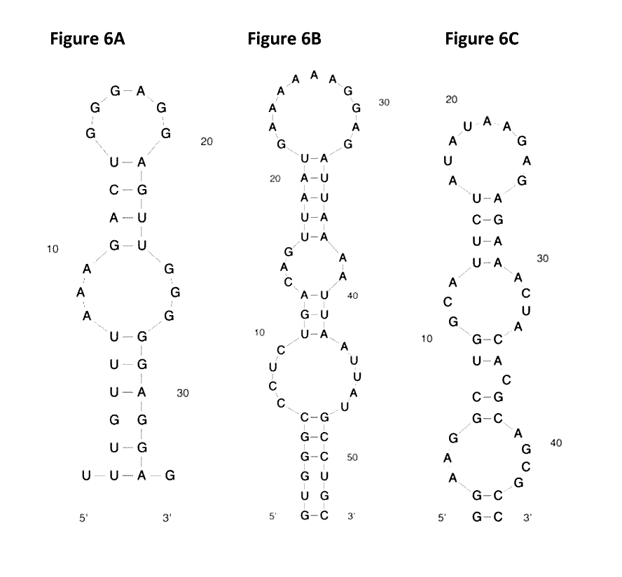

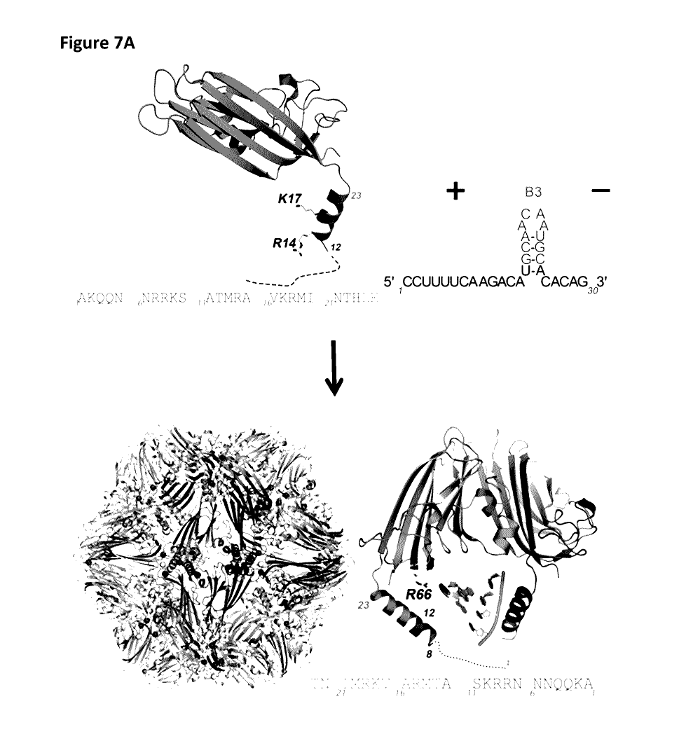

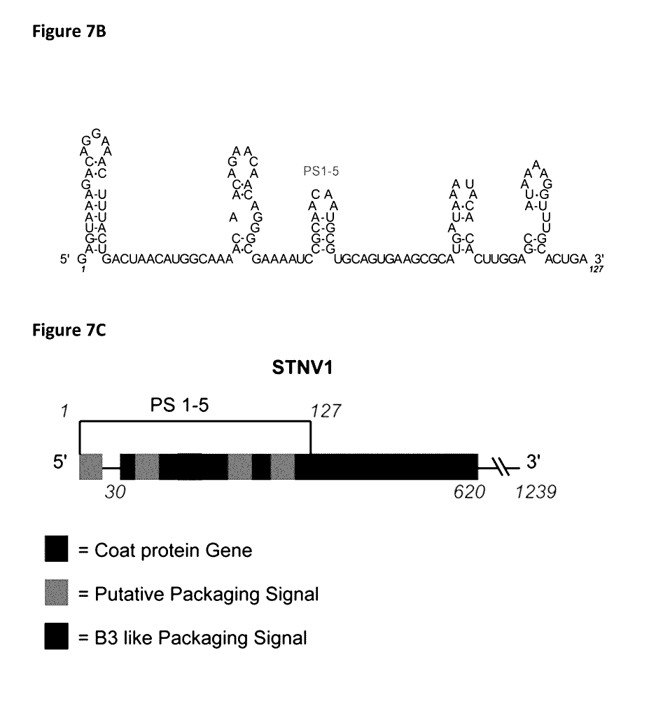

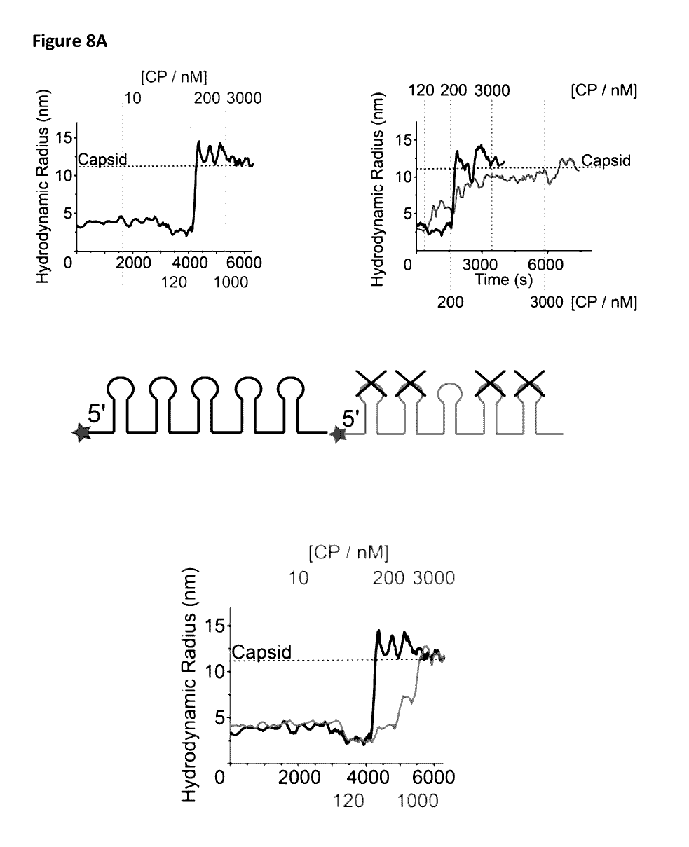

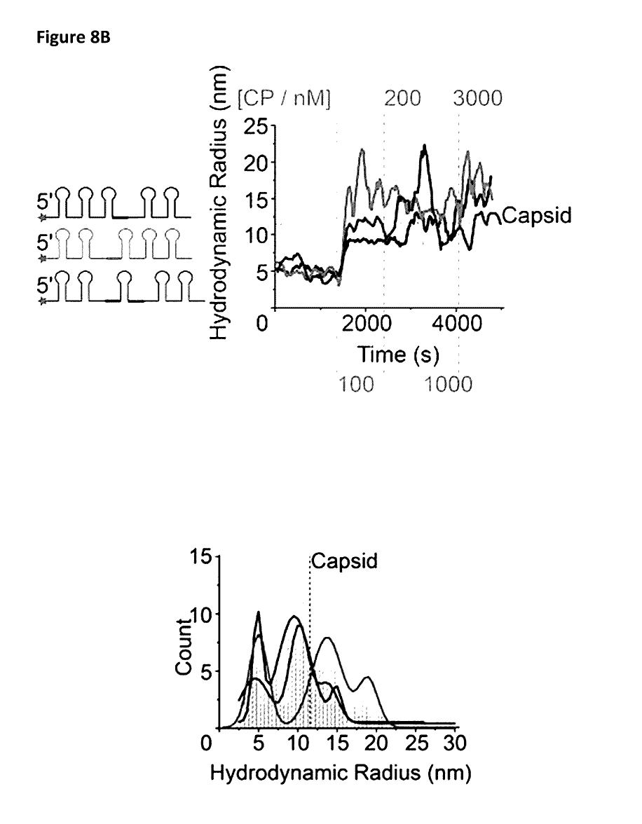

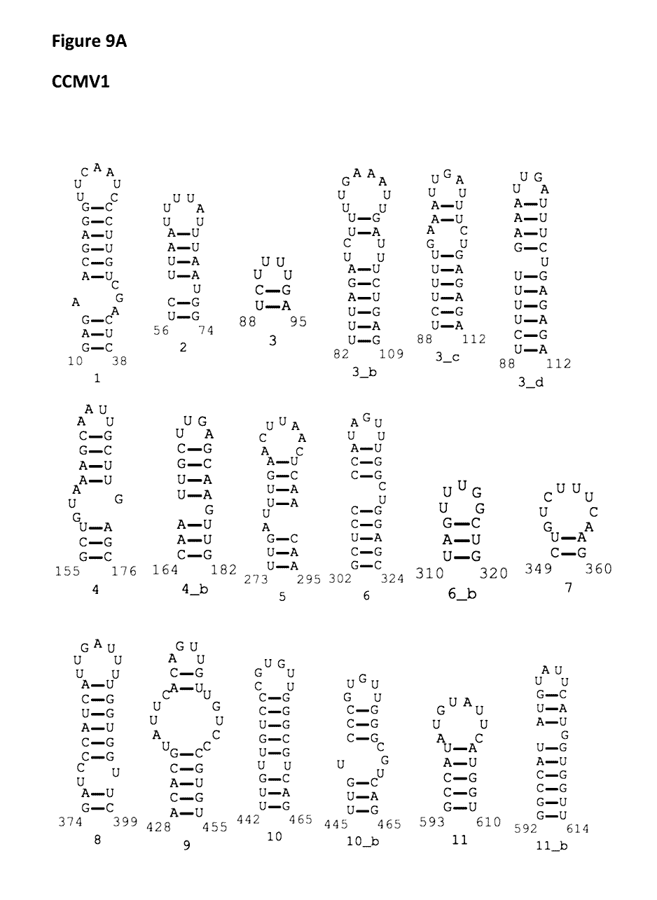

TABLE 25
Sequences for HBV PS (FIG. 6)

| SEQ ID | | | |
|---|---|---|---|
| NO: | | | Sequence |
| 142 | 1722 | 1756 | UUUGUUUAAAGACUGGGAGGAGUUGGGGGAGGAG |
| 143 | 2583 | 2636 | GUGGGCCCUCUGACAGUUAAUGAAAAAAGGAGAU |
| | | | UAAAAUUAAUUAUGCCUGC |
| 144 | 2761 | 2804 | GGAAGGCUGGCAUUCUAUAUAAGAGAGAAACUAC |
| | | | ACGCAGCGCC |

### REFERENCES

1. Borodavka, A., Tuma, R. & Stockley, P. G. (2012) Evidence that Viral RNAs have Evolved for Efficient, Two-stage Packaging. Proceedings Of The National Academy Of Sciences Of The United States Of America, 109, 15769-15774.
2. Borodavka A, Tuma R, Stockley P G. (2013) A two-stage mechanism of viral RNA compaction revealed by single molecule fluorescence. RNA Biol. 10(4), 481-9.
3. Dykeman et al, for submission to PNAS
4. Bunka D. H. J., Lane, S. W., Lane, C. L., Dykeman, E. C., Ford, R. J., Barker, A. M., Twarock, R., Phillips, S. E. V. & Stockley, P. G. (2011) Degenerate RNA Packaging Signals in the Genome of Satellite Tobacco Necrosis Virus: Implications for the Assembly of a T=1 Capsid. Journal of Molecular Biology, 413, 51-65.
5. Robert J. Ford, Amy M. Barker, Saskia E. Bakker, Robert H Coutts, Neil A. Ranson, Simon E. V. Phillips, Arwen R. Pearson & Peter G. Stockley. (2013) Sequence-specific, RNA-protein interactions overcome electrostatic barriers preventing assembly of Satellite Tobacco Necrosis Virus coat protein. J Mol. Biol. 425, 1050-64.
6. Dent, K. C., Thompson, R., Barker, A. M., Barr, J. N., Hiscox, J. A., Stockley, P. G. & Ranson, N. A. (2013). The asymmetric structure of an icosahedral virus bound to its receptor suggests a mechanism for genome release. Structure. doi:pii: S0969-2126(13)00194-9. 10.1016/j.str.2013.05.012.
7. Eric C. Dykeman, Peter G. Stockley and R. Twarock. Identification of dispersed, cryptic packaging signals in two viral RNA genomes reveals a conserved assembly mechanism. JMB doi:pii: S0022-2836(13)00365-3.
8. S. F. Altschul and B. W. Erickson, A Nonlinear measure of subalignment similarity and its significance levels Bul. Math Biol. 48 617-632 (1986)
9. Zuker, M. (2003). “Mfold web server for nucleic acid folding and hybridization prediction.” Nucleic Acids Res 31(13): 3406-15.
10. J. Andrew Berglund, Bruno Charpentier and Michael Rosbash (1997) A high affinity binding site for the HIV-1 nucleocapsid. Protein, Nucleic Acids Research 25, 1042-1049.
11. Jared L. Clever, Randy A. Taplitz, Michael A. Lochrie, Barry Polisky, and Tristram G. Parslow (2000) A Heterologous, High-Affinity RNA Ligand for Human Immunodeficiency Virus Gag Protein Has RNA Packaging Activity. J. Virol. 74, 541-546.
12 Robert J. Fisher et al (1998) Sequence-Specific Binding of Human Immunodeficiency Virus Type 1 Nucleocapsid Protein to Short Oligonucleotides. J. Virol. 72, p. 1902-1909.
13 J. Stephen Lodmell, Chantal Ehresmann, Bernard Ehresmann, and Roland Marquet (2000) Convergence of natural and artificial evolution on an RNA loop-loop interaction: The HIV-1 dimerization initiation site, RNA 6:1267-1276.
14 Andrew C. Paoletti, Michael F. Shubsda, Bruce S. Hudson,* and Philip N. Borer (2002) Affinities of the Nucleocapsid Protein for Variants of SL3 RNA in HIV-1, Biochemistry 41, 15423-15428.
15 Yi Qiong Yuan, Deborah J. Kerwood, Andrew C. Paoletti, Michael F. Shubsda, and Philip N. Borer (2003) Stem of SL1 RNA in HIV-1: Structure and Nucleocapsid Protein Binding for a 1×3 Internal Loo, Biochemistry 42, 5259-5269.
16 Joseph A Webb et al (2013) Distinct binding interactions of HIV-1 Gag to Psi and non-Psi RNAs: Implications for viral genomic RNA packaging, RNA 19:1078-1088
17 Joseph M. Watts, Kristen K. Dang, Robert J. Gorelick, Christopher W. Leonard, Julian W. Bess Jr, Ronald Swanstrom, Christina L. Burch & Kevin M. Weeks (2009) Architecture and secondary structure of an entire HIV-1 RNA genome. Nature 460, 711-716.

1. An anti-viral agent effective in controlling the formation of the viral capsid of an RNA virus wherein said agent is a nucleic acid stem-loop structure and comprises:
i) a nucleic acid loop domain comprising one or more nucleotide bases comprising a nucleotide binding motif for one or more capsid assembly domains in a viral capsid protein; and
ii) a nucleic acid stem domain wherein the stem domain is at least two nucleotide bases in length which over all or part of its length forms a double-stranded region by intramolecular complementary base pairing, wherein said anti-viral agent inhibits the formation of the viral capsid.

2. The agent according to claim 1, wherein said loop domain comprises at least 4 nucleotides.

3. The agent according to claim 1, wherein said loop domain comprises between 4 and 8 nucleotides.

4. The agent according to claim 1, wherein said stem domain comprises at least 2 nucleotides wherein at least one nucleotide is base paired with a complementary base.

5. The agent according to claim 1, wherein said stem domain comprises between 2 and 13 nucleotides which are base paired by intramolecular complementary base paring.

6. The agent according to claim 1, wherein said loop domain comprises at least one uracil base.

7. The agent according to claim 6, wherein said loop domain comprises at least 2, 3 or 4 uracil bases.

8. The agent according to claim 1, wherein said RNA virus is an animal virus.

9. The agent according to claim 8, wherein said animal RNA virus is a human virus.

10. The agent according to claim 9, wherein said human virus is a hepatitis virus.

11. The agent according to claim 10, wherein said hepatitis virus is hepatitis B virus [HBV] or hepatitis C virus [HCV].

12. The agent according to claim 11, wherein said nucleic acid based anti-viral agent comprises:
i) a nucleic acid loop domain comprising 5 to 12 nucleotide bases comprising an A-G nucleotide base rich binding motif for one or more HBV capsid assembly domains in a HBV capsid protein; and
ii) a nucleic acid stem domain wherein the stem domain comprises 4 to 30 nucleotides in length which over all or part of its length forms a double-stranded region by intramolecular complementary base pairing, wherein said anti-viral agent inhibits the formation of the HBV capsid.

13. The agent according to claim 12, wherein said binding motif comprises an A-G nucleotide base rich loop motif separated by 3 to 5 nucleotide base pairs from a bulge region containing A and/or G nucleotide base[s].

14. The agent according to claim 12, wherein said stem domain comprises between 3 and 5 nucleotide base pairs, followed by a bulge region that preferentially contains A and G nucleotide bases.

15. The agent according to claim 12, wherein said nucleic acid based anti-viral agent comprises or consists of a nucleotide sequence set forth in SEQ ID NO: 142, 143 or 144.

16. The agent according to claim 11, wherein said nucleic acid based anti-viral agent comprises:
i) a nucleic acid loop domain comprising 5 to 11 nucleotide bases comprising a G-rich nucleotide binding motif, preferentially containing the nucleotide bases GGG and a G and/or A nucleotide base at the start and/or end of the loop domain, for one or more HCV capsid assembly domains in a HCV capsid protein; and
ii) a nucleic acid stem domain wherein the stem domain is 14 to 23 nucleotides in length which over all or part of its length forms a double-stranded region by intramolecular complementary base pairing, wherein said anti-viral agent inhibits the formation of the HCV capsid.

17. The agent according to claim 16, wherein said binding motif comprises a G-rich nucleotide base motif.

18. The agent according to claim 17, wherein said binding motif comprises GGG and an A and/or G nucleotide base at the start and/or end of the loop portion.

19. The agent according to claim 16, wherein said nucleic acid based anti-viral agent comprises or consists of a nucleotide sequence set forth in SEQ ID NO: 184, 185, 186, 187, 188, 189, 190 or 191.

20. The agent according to claim 9, wherein said human virus is human parechovirus.

21. The agent according to claim 20, wherein said nucleic acid based anti-viral agent comprises:
i) a nucleic acid loop domain comprising 4 to 6 nucleotide bases comprising a binding motif for one or more parechoviral capsid assembly domains in a parechoviral capsid protein; and
ii) a nucleic acid stem domain 1 stem domain comprises 13 to 35 nucleotides which over all or part of its length forms a double-stranded region by intramolecular complementary base pairing, wherein said anti-viral agent inhibits the formation of the parechoviral capsid.

22. The agent according to claim 21, wherein said binding motif comprises a poly-U nucleotide base motif with a single purine, preferably a G nucleotide base.

23. The agent according to claim 21, wherein said stem domain comprises between 2 and 5 base pairs adjacent to a bulge region which is preferentially pyrimidine rich.

24. The agent according to claim 21, wherein said nucleic acid based anti-viral agent comprises or consists of a nucleotide sequence set forth in SEQ ID NO: 1, 2, 3, 4, 5, 6, 7, 8, 9, 10, 11, 12, 13 14, 578, 579, 580, 581, 582, 583, 584, 585, 586, 587, 588, 589, 590, 591, 592, 593, 594, 595, 596, 597, 598, 599, 600 or 601.

25. The agent according to claim 9, wherein said human virus is a human immune deficiency virus [HIV].

26. The agent according to claim 25, wherein said nucleic acid based anti-viral agent comprises:
i) a nucleic acid loop domain comprising 6 to 8 nucleotide bases comprising one or two of the binding motifs comprising at least one A nucleotide base for one or more Human Immunodeficiency Virus [HIV] capsid assembly domains in a HIV capsid protein; and
ii) a nucleic acid stem domain wherein the stem domain is 4, 5, 6, 7 or 8 nucleotides in length which over all or part of its length forms a double-stranded region by intramolecular complementary base pairing, wherein said anti-viral agent inhibits the formation of the HIV capsid.

27. The agent according to claim 26, wherein said binding motif comprises a nucleic acid loop with one or two of the nucleotide base motifs selected from the group consisting of: [AAX . . . X], [X . . . XAA], [CAX . . . X], [X . . . XCA], [ACX . . . X], and [X . . . XAC], wherein X is any nucleotide base and further wherein the nucleotide bases AA, CA, or AC is separated by one or more nucleotide bases.

28. The agent according to claim 26, wherein said nucleic acid based anti-viral agent comprises or consists of a nucleotide sequence as set forth in SEQ ID NO: 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 573, 574, 575, 576 or 577.

29. The agent according to claim 1, wherein said RNA virus is a plant RNA virus.

30. The agent according to claim 29, wherein said plant virus is Turnip Crinkle Virus.

31. The agent according to claim 30, wherein said nucleic acid based anti-viral agent comprises:
i) a nucleic acid loop domain comprising 7 to 12 nucleotide bases comprising a nucleotide binding motif for one or more Turnip Crinkle Virus [TCV] capsid assembly domains in a TCV capsid protein; and
ii) a nucleic acid stem domain wherein the stem domain is 24 to 32 nucleotide bases in length which over all or part of its length forms a double-stranded region by intramolecular complementary base pairing, wherein said anti-viral agent inhibits the formation of the TCV capsid.

32. The agent according to claim 31, wherein said nucleotide binding motif comprises a purine rich binding motif; preferably said motif comprises the nucleotide bases GGG or AAA.

33. The agent according to claim 31, wherein said stem domain comprises at least one purine rich bulge of three or more nucleotide bases.

34. The agent according to any one of claim 31, wherein said nucleic acid based anti-viral agent comprises or consists of a nucleotide sequence set forth in SEQ ID NO: 54, 55, 56, 57, 58, 59, 60, 61, 62, 63, 64, 65, 66, 67, 68, or 69.

35. The agent according to any one of claim 31, wherein said nucleic acid based anti-viral agent comprises or consists of a nucleotide sequence set forth in SEQ ID NO: 472, 473, 474 or 475.

36. The agent according to claim 29, wherein said plant virus is Cowpea Chlorotic Mottle Virus 1, 2 or 3.

37. The agent according to claim 36, wherein said nucleic acid based anti-viral agent comprises:
i) a nucleic acid loop domain comprising 4 to 8 nucleotide bases comprising a binding motif with at least one U nucleotide base for one or more Cowpea Chlorotic Mottle Virus 1 [CCMV1] capsid assembly domains in a CCMV1 capsid protein; and
ii) a nucleic acid stem domain wherein the stem domain is 8 to 31 nucleotide bases in length which over all or part of its length forms a double-stranded region by intramolecular complementary base pairing, wherein said anti-viral agent inhibits the formation of the CCMV1 capsid.

38. The agent according to claim 37, wherein said binding motif comprises the sequence UUXX or XXUU, wherein X is any nucleotide base.

39. The agent according to claim 37, wherein said nucleic acid based anti-viral agent comprises or consists of a nucleotide sequence set forth in SEQ ID NO: 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369 or 370.

40. The agent according to claim 36, wherein said nucleic acid based anti-viral agent comprises:
i) a nucleic acid loop domain comprising 4 to 8 nucleotide bases comprising a binding motif comprising at least one U nucleotide base for one or more Cowpea Chlorotic Mottle Virus 2 [CCMV2] capsid assembly domains in a CCMV2 capsid protein; and
ii) a nucleic acid stem domain wherein the stem domain is 8 to 32 nucleotides in length which over all or part of its length forms a double-stranded region by intramolecular complementary base pairing, wherein said anti-viral agent inhibits the formation of the CCMV2 capsid.

41. The agent according to claim 40, wherein said binding motif comprises the sequence UUXX or XXUU wherein X is any nucleotide base.

42. The agent according to claim 40, wherein said nucleic acid based anti-viral agent comprises or consists of a nucleotide sequence set forth in SEQ ID NO: 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, or 429.

43. The agent according to claim 36, wherein said nucleic acid based anti-viral agent comprises:
i) a nucleic acid loop domain comprising 4 to 8 nucleotide bases comprising a binding motif comprising at least one U nucleotide base for one or more Cowpea Chlorotic Mottle Virus 3 [CCMV3] capsid assembly domains in a CCMV3 capsid protein; and
ii) a nucleic acid stem domain wherein the stem domain is 8 to 35 nucleotides in length which over all or part of its length forms a double-stranded region by intramolecular complementary base pairing, wherein said anti-viral agent inhibits the formation of the CCMV3 capsid.

44. The agent according to claim 43, wherein In a preferred embodiment of the invention said binding motif comprises the sequence the sequence UUXX or XXUU wherein X is any nucleotide base.

45. The agent according to claim 43, wherein said nucleic acid based anti-viral agent comprises or consists of a nucleotide sequence set forth in SEQ ID NO: 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470 or 471.

46. The agent according to claim 43 wherein said nucleic acid based anti-viral agent comprises or consists of a nucleotide sequence set forth in SEQ ID NO: 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, or 113.

47. The agent according to claim 29, wherein said plant virus is Brome Mosaic Virus 1, 2, or 3.

48. The agent according to claim 47, wherein said nucleic acid based anti-viral agent comprises:
i) a nucleic acid loop domain comprising 4 to 8 nucleotide bases comprising a binding motif comprising at least one U nucleotide base for one or more Brome Mosaic Virus 1 [BMV1] capsid assembly domains in a BMV1 capsid protein; and
ii) a nucleic acid stem domain wherein the stem domain is 9 to 34 nucleotides in length which over all or part of its length forms a double-stranded region by intramolecular complementary base pairing, wherein said anti-viral agent inhibits the formation of the BMV1 capsid.

49. The agent according to claim 48, wherein said binding motif comprises the sequence UUXX or XXUU wherein X is any nucleotide base.

50. The agent according to claim 48, wherein said nucleic acid based anti-viral agent comprises or consists of a nucleotide sequence set forth in SEQ ID NO: 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182 or 183.

51. The agent according to claim 47, wherein said nucleic acid based anti-viral agent comprises:
i) a nucleic acid loop domain comprising 4 to 8 nucleotide bases comprising a binding motif comprising at least one U nucleotide base for one or more Brome Mosaic Virus 2 [BMV2] capsid assembly domains in a BMV2 capsid protein; and
ii) a nucleic acid stem domain wherein the stem domain is 8 to 35 nucleotides in length which over all or part of its length forms a double-stranded region by intramolecular complementary base pairing, wherein said anti-viral agent inhibits the formation of the BMV2 capsid.

52. The agent according to claim 51, wherein said binding motif comprises the sequence UUXX or XXUU wherein X is any nucleotide base.

53. The agent according to claim 51, wherein said nucleic acid based anti-viral agent comprises or consists of a nucleotide sequence set forth in SEQ ID NO: 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255 or 256.

54. The agent according to claim 47, wherein said nucleic acid based anti-viral agent comprises:
i) a nucleic acid loop domain comprising 4 to 8 nucleotide bases comprising a binding motif comprising at least one U nucleotide base for one or more Brome Mosaic Virus 3 [BMV3] capsid assembly domains in a BMV3 capsid protein; and
ii) a nucleic acid stem domain wherein the stem domain is 9 to 38 nucleotides in length which over all or part of its length forms a double-stranded region by intramolecular complementary base pairing, wherein said anti-viral agent inhibits the formation of the BMV3 capsid.

55. The agent according to claim 54, wherein said binding motif comprises the sequence UUXX or XXUU wherein X is any nucleotide base.

56. The agent according to claim 54, wherein said nucleic acid based anti-viral agent comprises or consists of a nucleotide sequence set forth in SEQ ID NO: 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294 or 295.

57. The agent according to claim 54, wherein said nucleic acid based anti-viral agent comprises or consists of a nucleotide sequence set forth in SEQ ID NO: 114, 115, 116, 117, 118, 119, 120, 121, 122, 123, 124, 125, 126, 127, 128, 129, 130, 131, 132, 133, 134, or 135.

58. The agent according to claim 78, wherein said nucleic acid based anti-viral agent comprises:
i) a nucleic acid loop domain comprising 4 to 6 nucleotide bases comprising a binding motif comprising at least one A nucleotide base for one or more Satellite Tobacco Necrosis Virus 1 [STNV-1] capsid assembly domains in an STNV-1 capsid protein; and
ii) a nucleic acid stem domain wherein the stem domain is 4 to 26 nucleotides in length which over all or part of its length forms a double-stranded region by intramolecular complementary base pairing, wherein said anti-viral agent inhibits the formation of the STNV 1 capsid.

59. The agent according to claim 78, wherein said nucleic acid based anti-viral agent comprises:
i) a nucleic acid loop domain comprising 4 to 6 nucleotide bases comprising a binding motif comprising at least one A nucleotide base for one or more Satellite Tobacco Necrosis Virus 2 [STNV-2] capsid assembly domains in an STNV-2 capsid protein; and
ii) a nucleic acid stem domain wherein the stem domain is 4 to 26 nucleotides in length which over all or part of its length forms a double-stranded region by intramolecular complementary base pairing, wherein said anti-viral agent inhibits the formation of the STNV-2 capsid.

60. The agent according to claim 78, wherein said nucleic acid based anti-viral agent comprises:
i) a nucleic acid loop domain comprising 4 to 6 nucleotide bases comprising a binding motif comprising at least one A nucleotide base in one or more Satellite Tobacco Necrosis Virus c [STNV-c] capsid assembly domains in an STNV-c capsid protein; and
ii) a nucleic acid stem domain wherein the stem domain is 4 to 26 nucleotides in length which over all or part of its length forms a double-stranded region by intramolecular complementary base pairing, wherein said anti-viral agent inhibits the formation of the STNV-c capsid.

61. The agent according to claim 58, wherein said binding motif comprises [AX . . . XA], [XAX . . . XA] or [AX . . . XAX], wherein X is any nucleotide base and further wherein each A nucleotide base is separated by at least one nucleotide base.

62. The agent according to claim 58, wherein said nucleic acid based anti-viral agent comprises or consists of a nucleotide sequence set forth in SEQ ID NO: 476, 477, 478, 479, 480, 481, 482, 483, 484, 485, 486, 487, 488, 489, 490, 491, 492, 493, 494, 495, 496, 497, 498, 499, 500, 501, 502, 503, 504 or 505.

63. The agent according to claim 59, wherein said nucleic acid based anti-viral agent comprises or consists of a nucleotide sequence set forth in SEQ ID NO: 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536 or 537.

64. The agent according to claim 60, wherein said nucleic acid based anti-viral agent comprises or consists of a nucleotide sequence set forth in SEQ ID NO: 538, 539, 540, 541, 542, 543, 544, 545, 546, 547, 548, 549, 550, 551, 552, 553, 554, 556, 557, 558, 559, 560, 561, 562, 563, 564, 565, 566, 567, 568, 569, 570, 571, or 572.

65. The agent according to any one of claim 1, wherein said nucleic acid based agent comprises modified nucleotides.

66. (canceled)

67. A pharmaceutical or plant protection product composition comprising the anti-viral agent of claim 1, and an excipient and/or carrier.

68. A combined pharmaceutical composition comprising the agent of claim 1, and one or more additional anti-viral agents different from said agent.

69.-71. (canceled)

72. A plant expression vector adapted for expression in a plant cell comprising the agent of claim 29.

73. A transgenic plant cell transfected with the expression vector according to claim 72.

74. A plant comprising the plant cell according to claim 73.

75. A method to screen for anti-viral agents that bind to one or more packaging signals and/or one or more viral capsid proteins comprising the steps:
i) providing a preparation comprising a combinatorial library of small molecular weight compounds and contacting said library with a preparation comprising:
a. a viral capsid protein or part thereof; or
b. a viral packaging signal;
ii) providing conditions sufficient to allow the binding of one or more compounds to either said viral capsid protein or viral packaging signal;
iii) selecting candidate agents that associate or bind either the viral capsid protein or viral packaging signal; and
iv) testing the activity of a selected compound for anti-viral activity.

76. A screening method for identification of nucleic acid based agents comprising one or more nucleotide sequences comprising a binding motif for one or more capsid assembly domains in a viral capsid protein comprising the steps:
i) forming a preparation comprising a viral capsid protein and a library of nucleic acid based agents;
ii) providing conditions suitable for specifically binding a nucleic acid based agent in (i) above with one or more capsid proteins;
iii) eluting capsid bound nucleic binding agents from said capsid protein[s];
iv) amplification of the eluted nucleic acid binding agents in (iii) above;
v) repeat steps (ii) to (iv) one or more times to enrich for said nucleic acid based agent[s]; and
vi) determine the sequence of the enriched nucleic acid based agent[s].

77. A method to determine one or more packaging signals in an RNA virus comprising the steps:
i) providing a nucleotide sequence of one or more nucleic acid binding agents selected by the method according to the invention;
ii) comparing the nucleotide sequence in (i) above with the genomic nucleotide sequence of an RNA virus to be assessed for the presence of a packaging signal;
iii) selecting a genomic RNA sequence based on a degree of similarity to the nucleotide sequence in (i) above; and optionally
iv) determining whether the selected genomic RNA sequence or part thereof binds the viral capsid protein of the RNA virus.

78. The agent according to claim 29, wherein said plant virus is Satellite Tobacco Necrosis Virus 1 (STNV-1), Satellite Tobacco Necrosis Virus 2 (STNV-2), or Satellite Tobacco Necrosis Virus c (STNV-c).
